(19) United States
(12) Patent Application Publication
(10) Pub. No.: US 2006/0162018 A1
(43) Pub. Date: Jul. 20, 2006

(54) Plant transcriptional regulators of disease resistance

(76) Inventors: Neil I. Gutterson, Oakland, CA (US); T. Lynne Reuber, San Mateo, CA (US); Jeffrey M. Libby, Cupertino, CA (US)

Correspondence Address: MORRISON & FOERSTER LLP, 425 MARKET STREET, SAN FRANCISCO, CA, 94105-2482, US

(21) Appl. No.: 10/559,441

(22) PCT Filed: Jun. 4, 2004
(86) PCT No.: PCT/US04/17768

§ 371 Date: Dec. 2, 2005

(30) Foreign Application Priority Data
Jun. 6, 2003 (US) 10/465,882

(51) Int. Cl. A01H 1/00; C12N 15/82

(52) U.S. Cl. 800/279; 435/468

(57) ABSTRACT

The invention relates to plant transcription factor polypeptides, polynucleotides that encode them, homologs from a variety of plant species, and methods of using the polynucleotides and polypeptides to produce transgenic plants having increased disease resistance or tolerance compared to a control plant. Sequence information related to these polynucleotides and polypeptides can also be used in bioinformatic search methods to identify related sequences and is also disclosed.

### FIELD OF THE INVENTION

The present invention relates to compositions and methods for increasing the tolerance or resistance of a plant to one or more pathogens.

### BACKGROUND OF THE INVENTION

In the broadest sense, the definition of plant disease includes anything that damages plant health. More commonly, plant disease refers to “biotic disease”, that is, the adverse effects of infectious pathogens that multiply on or within a plant and have the potential to spread to other plants. Plant pathogen injury may affect any part of a plant, and include defoliation, chlorosis, stunting, lesions, loss of photosynthesis, distortions, necrosis, and death. All of these symptoms ultimately result in yield loss in commercially valuable species.

Plant disease management is a considerable expense in crop production worldwide. Despite this expenditure, plant diseases significantly reduce worldwide crop productivity. Fungicides, insecticides, and anti-bacterial treatments are expensive, and their application poses both environmental and health risks.

The use of genetic engineering technologies to enhance the natural ability of plants to tolerate or resist pathogen attack holds great potential for enhancing yields while reducing chemical inputs. Manipulation of valuable traits such as disease tolerance or resistance may be achieved by altering the expression of critical regulatory molecules that are often conserved between diverse plant species. Related conserved regulatory molecules may be originally discovered in a model system (for example, in Arabidopsis) and homologous, functional molecules then discovered in other plant species. Regulatory molecules include transcription factors—proteins that increase or decrease (induce or repress) the rate of transcription of a particular gene or sets of genes. These proteins modulate cellular processes, which results in differential levels of gene expression at various developmental stages, in different tissues and cell types, and in response to different exogenous (e.g., environmental) and endogenous stimuli throughout the life cycle of the organism. Transformed and transgenic plants that comprise cells having altered levels of at least one selected transcription factor, for example, may possess advantageous or desirable traits. Strategies for manipulating traits by altering a plant cell's transcription factor content can therefore result in plants and crops with new and/or improved commercially valuable properties, including broad-spectrum resistance. Although enhanced disease resistance caused by the overexpression of defense gene regulators or signal transduction components has been reported previously (for example, see Cao and Dong (1998) Proc. Natl. Acad. Sci. USA 95: 6531-6653; Century et al. (1997) Science 278: 1963-1965; and Oldroyd and Staskawicz (1998) Proc. Natl. Acad. Sci. USA 95: 10300-10305), expression of these regulatory genes did not result in broad spectrum resistance to both biotrophic and necrotrophic pathogens.

The transcription factor G28 (GenBank accession number AB008103; SEQ ID NO: 2) is a downstream component of an ethylene (ET) response pathway (Fujimoto et al. (2000) Plant Cell 12: 393-404) and is a member of a family of structurally related transcription factors that contain ERF (ethylene response factor) domains that activate target genes containing a so-called ethylene responsive element (ERE; GCC box; Chao et al; (1997) Cell 89: 1133-1144; Ohme-Takagi et al. (1995) Plant Cell 7: 173-182; Solano and Ecker et al. (1998) Curr. Opin. Plant Biol. 1, 393-398; Solano et al. (1998) Genes Dev. 12: 3703-3714; Stepanova et al. (2000) Curr. Opin. Plant Biol. 3: 353-360). The ERF domain that binds the ERE is a novel DNA binding element found only in plants. In addition to G28, the tomato ERF domain containing proteins Pti4, Pti5 and Pti6 have been implicated in a defense response pathway that acts downstream of the tomato resistance gene PTO (Gu et al. (2000) Plant Cell 12: 771-786; Jia and Martin (1999) Plant Mol. Biol. 40: 455-465; Thara et al. (1999) Plant J. 20: 475-483; Zhou et al. (1997) EMBO J. 16: 3207-3218). Pti4, in particular, is a relatively close homolog of AtERF1 and may function similarly to AtERF1. Indeed, recent work has shown that over-expression of Pti4 in transgenic Arabidopsis plants leads to enhanced resistance to E. orontii, similar to the resistance observed in Arabidopsis plants overexpressing G28 (Gu et al. (2002) Plant Cell 14, 817-831).

We have identified polynucleotides encoding transcription factors, including G28 and related sequences such as G3430 (SEQ ID NO: 9), paralogs and orthologs, developed numerous transgenic plants using these polynucleotides, and analyzed the plants for a disease resistance or tolerance. ID so doing, we have identified important polynucleotide and polypeptide sequences for producing commercially valuable plants and crops as well as the methods for making them and using them. Other aspects and embodiments of the invention are described below and can be derived from the teachings of this disclosure as a whole.

### SUMMARY OF THE INVENTION

The present invention pertains to recombinant polynucleotides encoding AP2 transcription factor polypeptides, specifically members of the G28 clade of transcription factor polypeptides. The sequences of the invention include polynucleotides and polypeptides derived from both dicots and monocots. The polypeptide sequences from monocots also contain a subsequence identified as Motif Y (exemplified by SEQ ID NO: 55). Sequences of the invention are considered to be those that are related to the transcription factor sequences of the invention and related sequences, produced artificially or found in plants, including, for example, polypeptide sequences that are substantially identical with the sequences found in the Sequence Listing, or polynucleotide sequences that hybridize over their full length to the polynucleotides in the Sequence Listing under stringent conditions. This includes SEQ ID NO: 9, G3430, or the complement of SEQ ID NO: 9. An example of stringent conditions given in this disclosure includes two wash steps of 6×SSC at 65° C., each step being 10-30 minutes in duration

The invention also pertains to transgenic monocot plants that contain the recombinant polynucleotide just described (that is, a polynucleotide encoding a member of the G28 clade of transcription factors that contains a Motif Y). These transgenic monocot plants have enhanced tolerance to fungal disease due to the expression of the recombinant polynucleotide. The transgenic monocotyledonous plants of the invention may also have increased tolerance or resistance, as compared to a control plant, to more than one pathogen. The pathogens may include, for example, diverse fungal pathogens including Botrytis, Fusarium, Erysiphe, and Sclerotinia.

The invention also pertains to a method for increasing the tolerance or resistance of a monocot plant to a pathogen. This is accomplished by providing an expression vector comprising:
(i) a polynucleotide sequence encoding a polypeptide comprising a Motif Y that is at least 82% identical to the Motif Y of SEQ ID NO: 55; and
(ii) regulatory elements flanking the polynucleotide sequence; these regulatory elements are able to control expression of said polynucleotide sequence in a target monocot plant.

The target monocot plant is then transformed with the expression vector to generate a transformed monocot plant capable of expressing the polynucleotide sequence. These steps thus increase the tolerance or resistance of the monocot plant to a pathogen, as compared to the tolerance or resistance level of a control plant.

The invention also pertains to a method for reducing yield loss in a monocot plant due to plant disease. The plant diseases may be caused by more than one type of pathogen, including fungal pathogens such as Botrytis, Fusarium, Erysiphe, and Sclerotinia. Similar to the method for increasing the tolerance or resistance of a monocot plant to a pathogen, noted above, the method steps include first providing an expression vector comprising:
(i) a polynucleotide sequence encoding a polypeptide comprising a Motif Y that is at least 82% identical to the Motif Y of SEQ ID NO: 55; and
(ii) regulatory elements flanking the polynucleotide sequence.

The target monocot plant is then transformed with the expression vector to generate a transformed monocot plant capable of expressing the polynucleotide sequence, and the plant is then grown. These steps increase the tolerance or resistance of the monocot plant to at least one pathogen, as compared to the tolerance or resistance level of a control plant that has the same disease and is infected by the same pathogen. This results in a smaller yield loss for the transformed monocot plant than the loss experienced by the control plant, when the transformed and non-transformed monocot plants are challenged with the same disease pathogen or pathogens.

### BRIEF DESCRIPTION OF THE SEQUENCE LISTING, TABLES, AND DRAWINGS

The Sequence Listing provides exemplary polynucleotide and polypeptide sequences of the invention. The traits associated with the use of the sequences are included in the Examples.

CD-ROM1 and CD-ROM2 are identical read-only memory computer-readable compact discs, and contain copies of the Sequence Listing in ASCII text format. The Sequence Listing is named “MBI0052PCT.ST25.txt” and is 97 kilobytes in size. The copies of the Sequence Listing on the CD-ROM discs are hereby incorporated by reference in their entirety.

FIG. 1 shows a conservative estimate of phylogenetic relationships among the orders of flowering plants (modified from Angiosperm Phylogeny Group (1998) Ann. Missouri Bot. Gard. 84: 1-49). Those plants with a single cotyledon (monocots) are a monophyletic lade nested within at least two major lineages of dicots; the eudicots are further divided into rosids and asterids.

Arabidopsis is a rosid eudicot classified within the order Brassicales; rice is a member of the monocot order Poales. FIG. 1 was adapted from Daly et al. (2001) Plant Physiol. 127: 1328-1333.

FIG. 2 shows a phylogenic dendogram depicting phylogenetic relationships of higher plant taxa, including clades containing tomato and Arabidopsis; adapted from Ku et al. (2000) Proc. Natl. Acad. Sci. USA 97: 9121-9126; and Chase et al. (1993) Ann. Missouri Bot. Gard. 80: 528-580.

FIGS. 3A-3G show an alignment of the G28 lade of transcription factor polypeptides (SEQ ID NO: 2) and polypeptide sequences encoded by polynucleotide sequences that are paralogous or orthologous to G28. The alignment was produced using Clustal X 1.81. The AP2 domains are indicated by the horizontal line at near the top of FIGS. 3D-3F. The monocot Motif Y subsequences appear in the boxes in FIGS. 3A and 3B.

FIG. 4 depicts a phylogenetic tree of several members of the G28 lade of transcription factor polypeptides, identified through BLAST analysis of proprietary (using corn, soy and rice genes) and public data sources (all plant species). This tree was generated as a Clustal X 1.81 alignment: MEGA2 tree, Maximum Parsimony, bootstrap consensus. Representative sequences of the G28 clade of transcription factor polypeptides may within the large box. The smaller box denotes representative members of the G3430 subclade.

### DETAILED DESCRIPTION OF EXEMPLARY EMBODIMENTS

As used herein and in the appended claims, the singular forms “a”, “an”, and “the” include the plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth

Definitions

“TDR” (in uppercase letters) refers generally to a Transcriptional regulator of Disease Resistance protein sequence of the present invention, including SEQ ID NOs: 2, 4, 8, 10, 12, 14, 16, 18, 20, 22, 24, 26, 28, 30, 32, 34, 36, 38, 40, 42, 60, paralogs, orthologs, equivalogs, and fragments thereof. The term “tdr” (in lowercase letters) refers generally to a polynucleotide sequence of the present invention, and includes SEQ ID NOs: 1, 3, 7, 9, 11, 13, 15, 17, 19, 21, 23, 25, 27, 29, 31, 33, 35, 37, 39, 41, 59, paralogs, orthologs, equivalogs, and fragments thereof.

“Tolerance” results from specific, heritable characteristics of a host plant that allow a pathogen to develop and multiply in the host while the host, either by lacking receptor sites for, or by inactivating or compensating for the irritant secretions of the pathogen, still manages to thrive or, in the case of crop plants, produce a good crop. Tolerant plants are susceptible to the pathogen but are not killed by it and generally show little damage from the pathogen (Agrios (1988) Plant Pathology, 3rd ed. Academic Press, N.Y., p. 129).

“Resistance”, also referred to as “true resistance”, results when a plant contains one or more genes that make the plant and a potential pathogen more or less incompatible with each other, either because of a lack of chemical recognition between the host and the pathogen, or because the host plant can defend itself against the pathogen by defense mechanisms already present or activated in response to infection (Agrios (1988) supra p. 125).

“Biologically active” refers to a protein having structural, immunological, regulatory, or chemical functions of a naturally occurring, recombinant or synthetic molecule.

“Complementary” refers to the natural hydrogen bonding by base pairing between purines and pyrimidines. For example, the sequence A-C-G-T (5′→3′) forms hydrogen bonds with its complements A-C-G-T (5′→3′) or A-C-G-U (5′→3′). Two single-stranded molecules may be considered partially complementary, if only some of the nucleotides bond, or “completely complementary” if all of the nucleotides bond. The degree of complementarity between nucleic acid strands affects the efficiency and strength of the hybridization and amplification reactions. “Fully complementary” refers to the case where bonding occurs between every base pair and its complement in a pair of sequences, and the two sequences have the same number of nucleotides.

A “conserved domain” or “conserved region” as used herein refers to a region in heterologous polynucleotide or polypeptide sequences where there is a relatively high degree of sequence identity between the distinct sequences.

With respect to polynucleotides encoding presently disclosed transcription factors, a conserved region is preferably at least 10 base pairs (bp) in length.

A “conserved domain” or “conserved region” as used herein refers to a region in heterologous polynucleotide or polypeptide sequences where there is a relatively high degree of sequence identity between the distinct sequences. An AP2 domain that is present in a member of AP2 transcription factor family is an example of a conserved domain. With respect to polynucleotides encoding presently disclosed transcription factors, a conserved domain is preferably at least 10 base pairs (bp) in length. A “conserved domain”, with respect to presently disclosed AP2 domains, refers to a domain within a transcription factor family that exhibits a higher degree of sequence homology, such as at least 60% sequence identity including conservative substitutions, and more preferably at least 75% sequence identity, and even more preferably at least 83%, or at least about 84%, or at least about 86%, or at least about 89%, or at least about 90%, or at least about 92%, or at least about 95%, or at least about 96% amino acid residue sequence identity to the conserved domain. A “conserved domain”, with respect to presently disclosed “Motif Y”, refers to a domain within a monocot AP2 transcription factor sequence that exhibits a high degree of sequence homology to the Motif Y found in SEQ ID NO: 55, having at least 82% sequence identity with the Motif Y found in SEQ ID NO: 55.

A fragment or domain can be referred to as outside a conserved domain, a consensus sequence, or a consensus DNA-binding site that is known to exist or that exists for a particular transcription factor class, family, or sub-family. In this case, the fragment or domain will not include the exact amino acids of a consensus sequence or consensus DNA-binding site of a transcription factor class, family or sub-family, or the exact amino acids of a particular transcription factor consensus sequence or consensus DNA-binding site. Furthermore, a particular fragment, region, or domain of a polypeptide, or a polynucleotide encoding a polypeptide, can be “outside a conserved domain” if all the amino acids of the fragment, region, or domain fall outside of a defined conserved domain(s) for a polypeptide or protein. Sequences having lesser degrees of identity but comparable biological activity are considered to be equivalents.

As one of ordinary skill in the art recognizes, conserved domains of transcription factors may be identified as regions or domains of identity to a specific consensus sequence (see, for example, Riechmann et al. (2000) Science 290: 2105-2110). In the subject invention, the plant transcription factors belong to the AP2 (APETALA2) domain transcription factor family (Riechmann and Meyerowitz (1998) Biol. Chem. 379: 633-646).

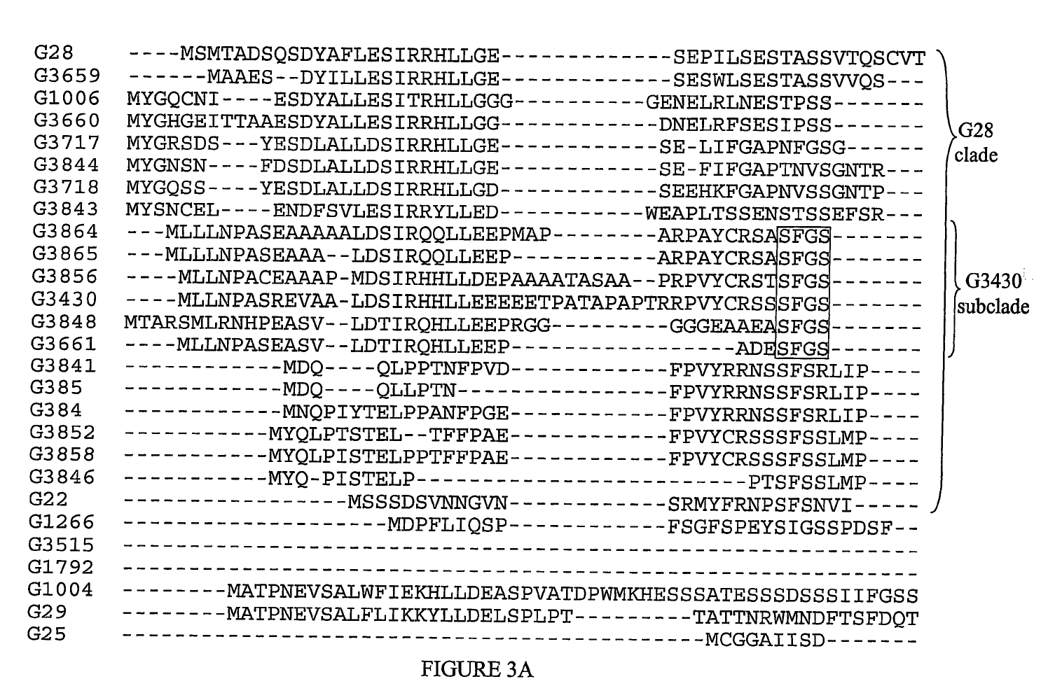

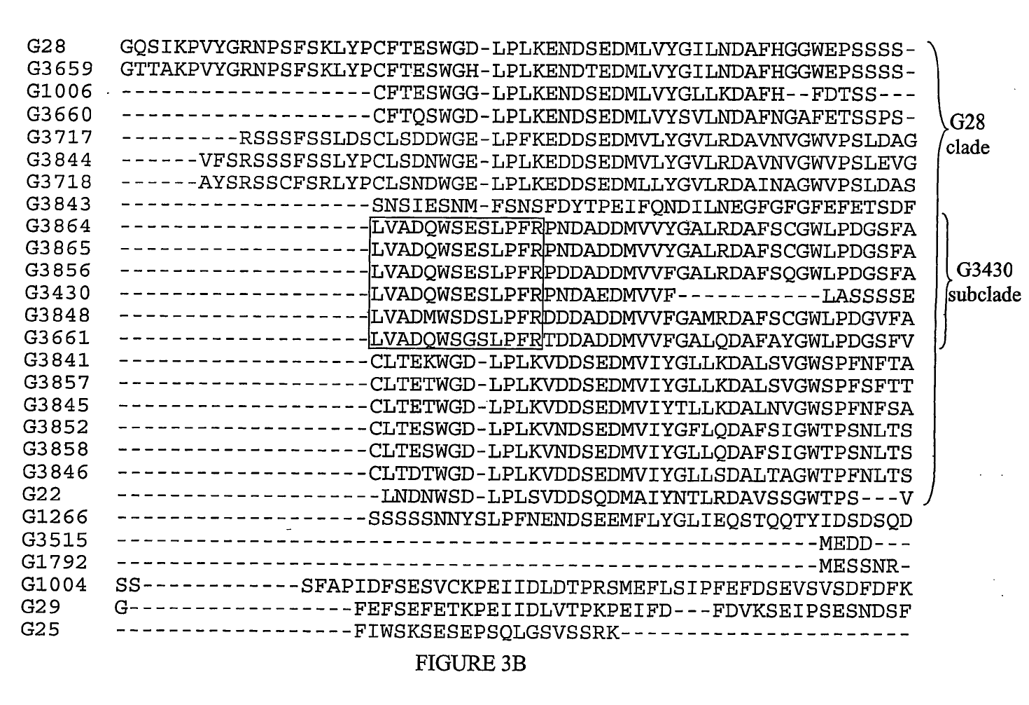

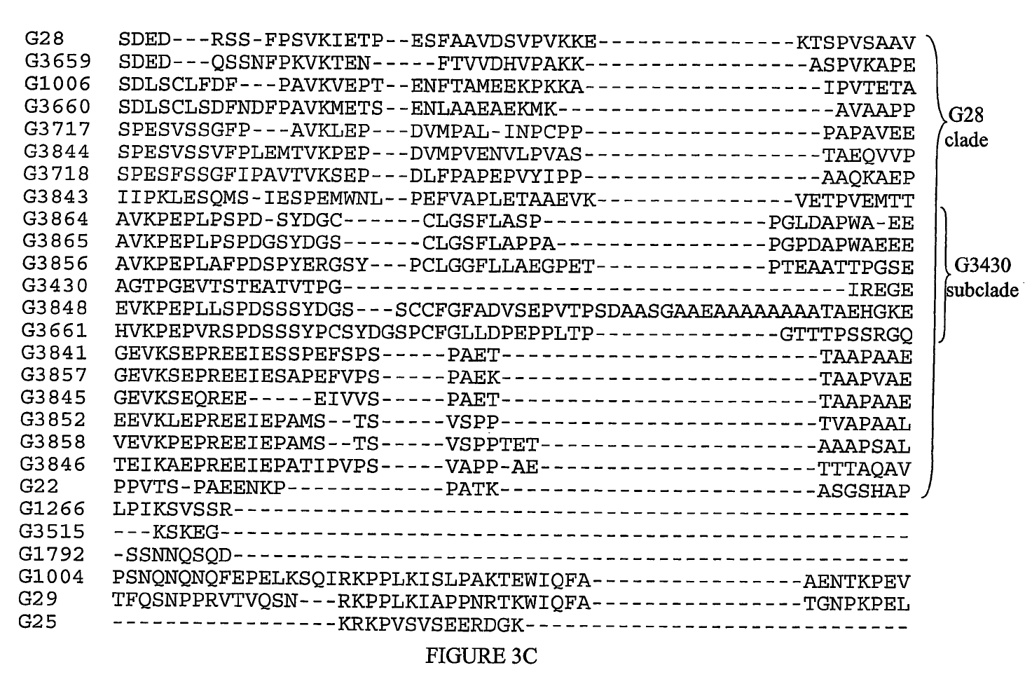

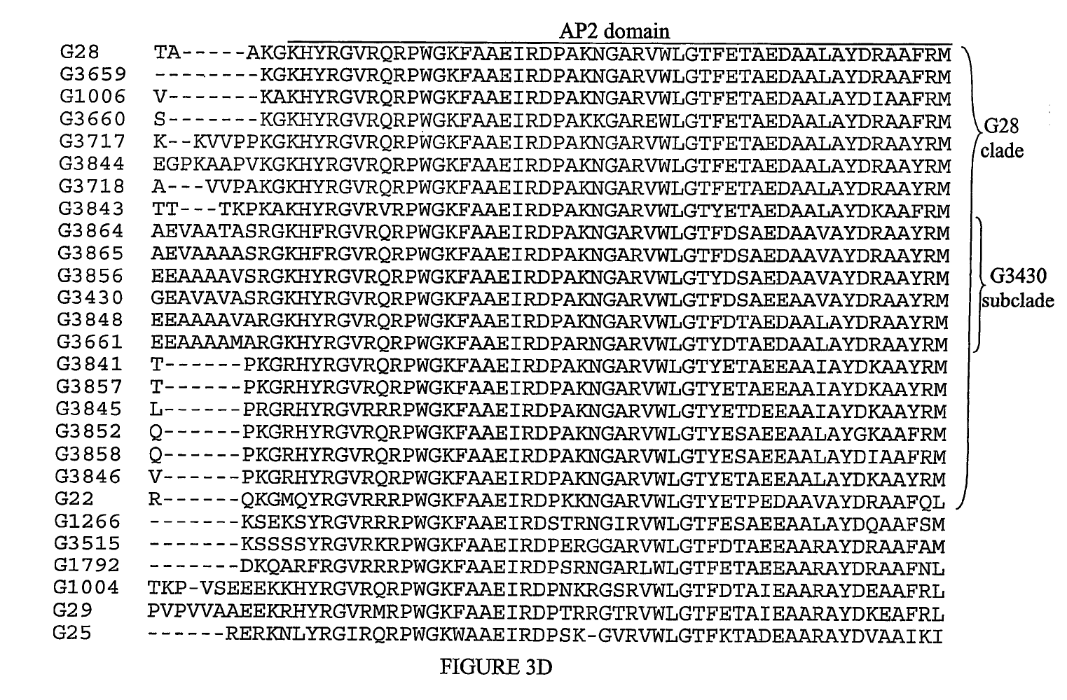

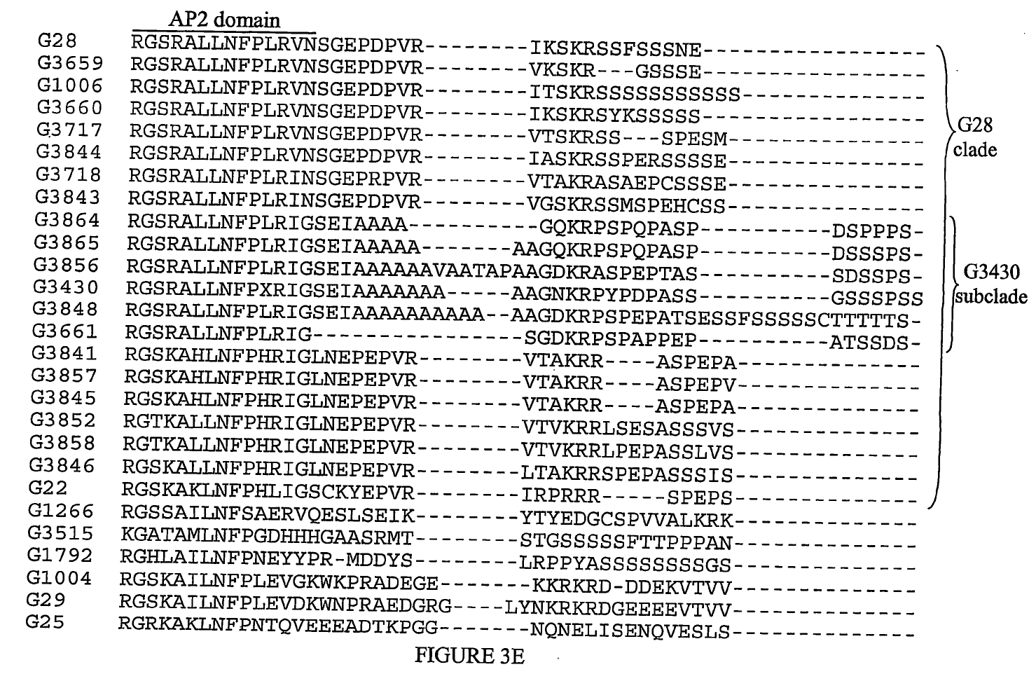

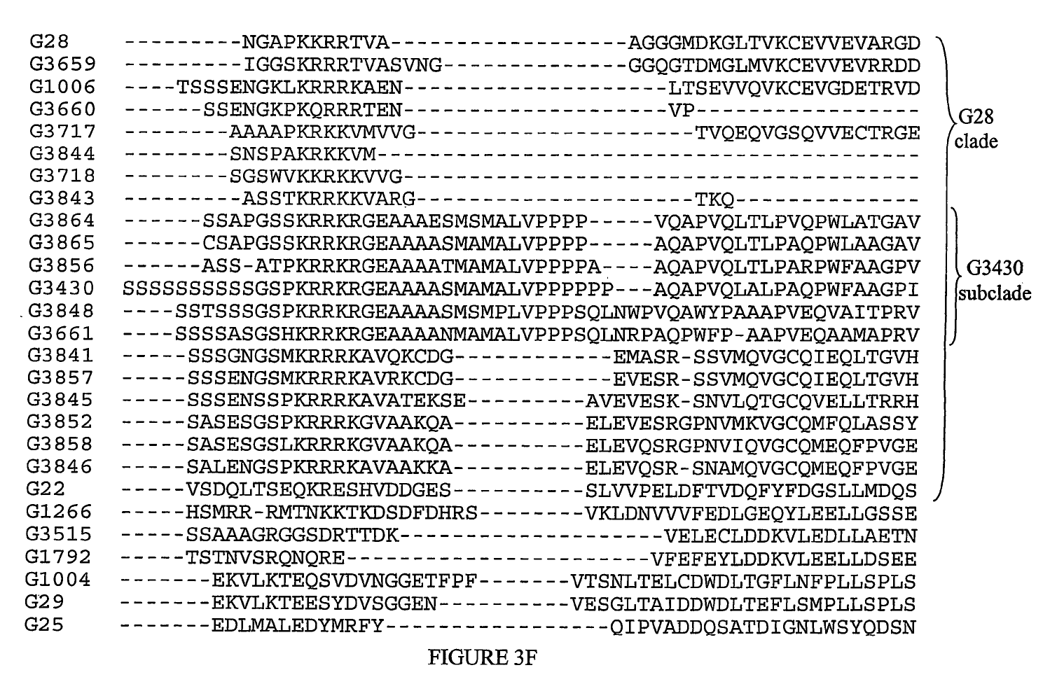

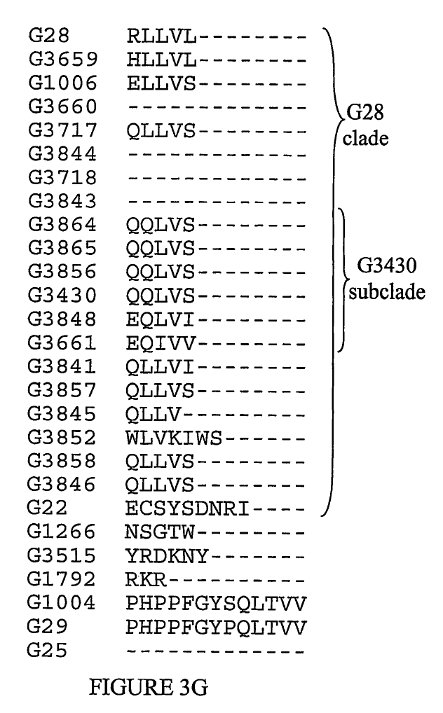

The conserved domains for some of the transcription factor polypeptides in the Sequence Listing are shown in FIGS. 3A-3B and 3D-3E. A comparison of the regions of the polypeptides in the Sequence Listing, or of those in FIGS. 3A-3B and 3D-3E, allows one of skill in the art to identify conserved domain(s) for any of the polypeptides listed or referred to in this disclosure.

“Derivative” refers to the chemical modification of a nucleic acid molecule or amino acid sequence. Chemical modifications can include replacement of hydrogen by an alkyl, acyl, or amino group or glycosylation, pegylation, or any similar process that retains or enhances biological activity or lifespan of the molecule or sequence.

“Fragment” with respect to a polynucleotide refers to a clone or any part of a nucleic acid molecule that retains a usable, functional characteristic. Fragments include oligonucleotides that may be used in hybridization or amplification technologies or in regulation of replication, transcription or translation.

“Fragment” with respect to polypeptide may also include subsequences of polypeptides and protein molecules, or a subsequence of the polypeptide. Fragments may have uses in that they may have antigenic potential. In some cases, the fragment or domain is a subsequence of the polypeptide that performs at least one biological function of the intact polypeptide in substantially the same manner, or to a similar extent, as does the intact polypeptide. For example, a polypeptide fragment can comprise a recognizable structural motif or functional domain such as a DNA-binding site or domain that binds to a DNA promoter region, an activation domain, or a domain for protein-protein interactions, and may initiate transcription. Fragments can vary in size from as few as 3 amino acids to the fall length of the intact polypeptide, but are preferably at least about 30 amino acids in length and more preferably at least about 60 amino acids in length. Exemplary polypeptide fragments are the first twenty consecutive amino acids of a mammalian protein encoded by the first twenty consecutive amino acids of the transcription factor polypeptides listed in the Sequence Listing.

Exemplary fragments also include fragments that comprise a conserved domain of a transcription factor. An example of such an exemplary fragment would include amino acid residues 45-61 of G3430 (SEQ ID NO: 10), as noted in FIGS. 3A-3B.

“Gene” or “gene sequence” refers to the partial or complete coding sequence of a gene, its complement, and its 5′ or 3′ untranslated regions. A gene is also a functional unit of inheritance, and in physical terms is a particular segment or sequence of nucleotides along a molecule of DNA (or RNA, in the case of RNA viruses) involved in producing a polypeptide chain. The polypeptide chain may be subjected to subsequent processing to obtain a functional protein or polypeptide. A gene may be isolated, partially isolated, or be found with an organism's genome. By way of example, a transcription factor gene encodes a transcription factor polypeptide, which may be functional or require processing to function as an initiator of transcription.

Operationally, genes may be defined by the cis-trans test, a genetic test that determines whether two mutations occur in the same gene and that may be used to determine the limits of the genetically active unit (Rieger et al. (1976) Glossary of Genetics and Cytogenetics: Classical and Molecular, 4th ed., Springer Verlag. Berlin). A gene generally includes regions preceding (“leaders”; upstream) and following (“trailers”; downstream) of the coding region. A gene may also include intervening, non-coding sequences, referred to as “introns”, located between individual coding segments, referred to as “exons”.

Most genes have an associated promoter region, a regulatory sequence 5′ of the transcription initiation codon (there are some genes that do not have an identifiable promoter). The function of a gene may also be regulated by enhancers, operators, and other regulatory elements.

“Homology” refers to sequence similarity between a reference sequence and at least a fragment of a newly sequenced clone insert or its encoded amino acid sequence.

“Identity” or “similarity” refers to sequence similarity between two polynucleotide sequences or between two polypeptide sequences, with identity being a more strict comparison. The phrases “percent identity” and “identity” refer to the percentage of sequence similarity found in a comparison of two or more polynucleotide sequences or two or more polypeptide sequences. “Sequence similarity” refers to the percent similarity in base pair sequence (as determined by any suitable method) between two or more polynucleotide sequences. Two or more sequences can be anywhere from 0-100% similar, or any integer value therebetween. Identity or similarity can be determined by comparing a position in each sequence that may be aligned for purposes of comparison. When a position in the compared sequence is occupied by the same nucleotide base or amino acid, then the molecules are identical at that position. A degree of similarity or identity between polynucleotide sequences is a function of the number of identical or matching nucleotides at positions shared by the polynucleotide sequences. A degree of identity of polypeptide sequences is a function of the number of identical amino acids at positions shared by the polypeptide sequences. A degree of homology or similarity of polypeptide sequences is a function of the number of amino acids at positions shared by the polypeptide sequences.

With regard to polypeptides, the terms “substantial identity” or “substantially identical” refers to sequences of sufficient structural similarity to the transcription factors in the Sequence Listing to produce similar function when expressed or overexpressed in a plant. In the present invention, similar functions confer increased tolerance or resistance to pathogens. Sequences that are at least 75% identical (e.g., in their AP2 domains) or at least 82% identical (e.g., in their Motif Ys) have been discovered and many of these are expected to have similar function as G28 and G3430 when expressed or overexpressed in plants. Thus, these sequences are considered to have substantial identity with G28 and G3430. Sequences having lesser degrees of identity but comparable biological activity are considered to be equivalents. The structure required to maintain proper functionality is related to the tertiary structure of the polypeptide. There are discreet domains and motifs within a transcription factor that must be present within the polypeptide to confer function and specificity. These specific structures are required so that interactive sequences will be properly oriented to retain the desired activity. “Substantial identity” may thus also be used with regard to subsequences, for example, motifs, that are of sufficient structure and similarity, being at least 75% identical or at least 82% identical to similar motifs in other related sequences so that each confers or is required for increased tolerance or resistance to pathogens.

“Alignment” refers to a number of nucleotide bases or amino acid residue sequences aligned by lengthwise comparison so that components in common (i.e., nucleotide bases or amino acid residues) may be visually and readily identified. The fraction or percentage of components in common is related to the homology or identity between the sequences. Alignments such as those of FIG. 3 may be used to identify conserved domains and relatedness within these domains. An alignment may suitably be determined by means of computer programs known in the art, such as MACVECTOR (Accelrys, Inc., San Diego, Calif.).

The terms “highly stringent” or “highly stringent condition” refer to conditions that permit hybridization of DNA strands whose sequences are highly complementary, wherein these same conditions exclude hybridization of significantly mismatched DNAs. Polynucleotide sequences capable of hybridizing under stringent conditions with the polynucleotides of the present invention may be, for example, variants of the disclosed polynucleotide sequences, including allelic or splice variants, or sequences that encode orthologs or paralogs of presently disclosed polypeptides. Nucleic acid hybridization methods are disclosed in detail by Kashima et al. (1985) Nature 313: 402-404, and Sambrook et al. (1989) Molecular Cloning: A Laboratory Manual, 2nd Ed., Vol. 1-3, Cold Spring Harbor Laboratory, Cold Spring Harbor, N.Y.; and by Haymes et al. (1985) Nucleic Acid Hybridization: A Practical Approach, IRL Press, Washington, D.C., which references are incorporated herein by reference.

In general, stringency is determined by the temperature, ionic strength, and concentration of denaturing agents (e.g., formamide) used in a hybridization and washing procedure (for a more detailed description of establishing and determining stringency, see below). The degree to which two nucleic acids hybridize under various conditions of stringency is correlated with the extent of their similarity. Thus, similar nucleic acid sequences from a variety of sources, such as within a plant's genome (as in the case of paralogs) or from another plant (as in the case of orthologs) that may perform similar functions can be isolated on the basis of their ability to hybridize with known transcription factor sequences. Numerous variations are possible in the conditions and means by which nucleic acid hybridization can be performed to isolate transcription factor sequences having similarity to transcription factor sequences known in the art and are not limited to those explicitly disclosed herein. Such an approach may be used to isolate polynucleotide sequences having various degrees of similarity with disclosed transcription factor sequences, such as, for example, transcription factors having 60% identity, or more preferably greater than about 70% identity, most preferably 72% or greater identity with disclosed transcription factors.

The term “equivalog” describes members of a set of homologous proteins that are conserved with respect to function since their last common ancestor. Related proteins are grouped into equivalog families, and otherwise into protein families with other hierarchically defined homology types. This definition is provided at the Institute for Genoinic Research (TIGR) world wide web (www) website, “tigr.org” under the heading “Terms associated with TIGRFAMs”.

The term “variant”, as used herein, may refer to polynucleotides or polypeptides that differ from the presently disclosed polynucleotides or polypeptides, respectively, in sequence from each other, and as set forth below.

With regard to polynucleotide variants, differences between presently disclosed polynucleotides and their variants are limited so that the nucleotide sequences of the former and the latter are closely similar overall and, in many regions, identical. The degeneracy of the genetic code dictates that many different variant polynucleotides can encode identical and/or substantially similar polypeptides in addition to those sequences illustrated in the Sequence Listing. Due to this degeneracy, differences between presently disclosed polynucleotides and variant nucleotide sequences may be silent in any given region or over the entire length of the polypeptide (i.e., the amino acids encoded by the polynucleotide are the same, and the variant polynucleotide sequence thus encodes the same amino acid sequence in that region or entire length of the presently disclosed polynucleotide. Variant nucleotide sequences may encode different amino acid sequences, in which case such nucleotide differences will result in amino acid substitutions, additions, deletions, insertions, truncations or fusions with respect to the similar disclosed polynucleotide sequences. These variations result in polynucleotide variants encoding polypeptides that share at least one functional characteristic (i.e., a presently disclosed transcription factor and a variant will confer at least one of the same functions to a plant).

Within the scope of the invention is a variant of a nucleic acid listed in the Sequence Listing, that is, one having a sequence that differs from the one of the polynucleotide sequences in the Sequence Listing, or a complementary sequence, that encodes a functionally equivalent polypeptide (i.e., a polypeptide having some degree of equivalent or similar biological activity) but differs in sequence from the sequence in the Sequence Listing, due to degeneracy in the genetic code.

“Allelic variant” or “polynucleotide allelic variant” refers to any of two or more alternative forms of a gene occupying the same chromosomal locus. Allelic variation arises naturally through mutation, and may result in phenotypic polymorphism within populations. Gene mutations may be “silent” or may encode polypeptides having altered amino acid sequences. “Allelic variant” and “polypeptide allelic variant” may also be used with respect to polypeptides, and in this case the terms refer to a polypeptide encoded by an allelic variant of a gene.

“Splice variant” or “polynucleotide splice variant” as used herein refers to alternative forms of RNA transcribed from a gene. Splice variation naturally occurs as a result of alternative sites being spliced within a single transcribed RNA molecule or between separately transcribed RNA molecules, and may result in several different forms of messenger RNA (mRNA) transcribed from the same gene. Thus, splice variants may encode polypeptides having different amino acid sequences, which, in the present context, will have at least one similar function in the organism (splice variation may also give rise to distinct polypeptides having different functions). “Splice variant” or “polypeptide splice variant” may also refer to a polypeptide encoded by a splice variant of a transcribed mRNA.

As used herein, “polynucleotide variants” may also refer to polynucleotide sequences that encode paralogs and orthologs of the presently disclosed polypeptide sequences. “Polypeptide variants” may refer to polypeptide sequences that are paralogs and orthologs of the presently disclosed polypeptide sequences.

“Modulates” refers to a change in activity (biological, chemical, or immunological) or lifespan resulting from specific binding between a molecule and either a nucleic acid molecule or a protein.

“Nucleic acid molecule” refers to a oligonucleotide, polynucleotide or any fragment thereof. It may be DNA or RNA of genomic or synthetic origin, double-stranded or single-stranded, and combined with carbohydrate, lipids, protein, or other materials to perform a particular activity such as transformation or form a useful composition such as a peptide nucleic acid (PNA).

“Polynucleotide” is a nucleic acid molecule comprising a plurality of polymerized nucleotides, e.g., at least about 15 consecutive polymerized nucleotides, optionally at least about 30 consecutive nucleotides, at least about 50 consecutive nucleotides. A polynucleotide may be a nucleic acid, oligonucleotide, nucleotide, or any fragment thereof. In many instances, a polynucleotide comprises a nucleotide sequence encoding a polypeptide (or protein) or a domain or fragment thereof. Additionally, the polynucleotide may comprise a promoter, an intron, an enhancer region, a polyadenylation site, a translation initiation site, 5′ or 3′ untranslated regions, a reporter gene, a selectable marker, or the like. The polynucleotide can be single stranded or double stranded DNA or RNA. The polynucleotide optionally comprises modified bases or a modified backbone. The polynucleotide can be, e.g., genomic DNA or RNA, a transcript (such as an mRNA), a cDNA, a PCR product, a cloned DNA, a synthetic DNA or RNA, or the like. The polynucleotide can be combined with carbohydrate, lipids, protein, or other materials to perform a particular activity such as transformation or form a useful composition such as a peptide nucleic acid (PNA). The polynucleotide can comprise a sequence in either sense or antisense orientations. “Oligonucleotide” is substantially equivalent to the terms amplimer, primer, oligomer, element, target, and probe and is preferably single stranded.

A “recombinant polynucleotide” is a polynucleotide that is not in its native state, e.g., the polynucleotide comprises a nucleotide sequence not found in nature, or the polynucleotide is in a context other than that in which it is naturally found, e.g., separated from nucleotide sequences with which it typically is in proximity in nature, or adjacent (or contiguous with) nucleotide sequences with which it typically is not in proximity. For example, the sequence at issue can be cloned into a vector, or otherwise recombined with one or more additional nucleic acid.

An “isolated polynucleotide” is a polynucleotide whether naturally occurring or recombinant, that is present outside the cell in which it is typically found in nature, whether purified or not. Optionally, an isolated polynucleotide is subject to one or more enrichment or purification procedures, e.g., cell lysis, extraction, centrifugation, precipitation, or the like.

A “polypeptide” is an amino acid sequence comprising a plurality of consecutive polymerized amino acid residues e.g., at least about 15 consecutive polymerized amino acid residues, optionally at least about 30 consecutive polymerized amino acid residues, at least about 50 consecutive polymerized amino acid residues. In many instances, a polypeptide comprises a polymerized amino acid residue sequence that is a transcription factor or a domain or portion or fragment thereof. A transcription factor can regulate gene expression and may increase or decrease gene expression in a plant. Additionally, the polypeptide may comprise 1) a localization domain, 2) an activation domain, 3) a repression domain, 4) an oligomerization domain, or 5) a DNA-binding domain, or the like. The polypeptide optionally comprises modified amino acid residues, naturally occurring amino acid residues not encoded by a codon, non-naturally occurring amino acid residues.

A “recombinant polypeptide” is a polypeptide produced by translation of a recombinant polynucleotide. A “synthetic polypeptide” is a polypeptide created by consecutive polymerization of isolated amino acid residues using methods well known in the art. An “isolated polypeptide,” whether a naturally occurring or a recombinant polypeptide, is more enriched in (or out of) a cell than the polypeptide in its natural state in a wild-type cell, e.g., more than about 5% enriched, more than about 10% enriched, or more than about 20%, or more than about 50%, or more, enriched, i.e., alternatively denoted: 105%, 110%, 120%, 150% or more, enriched relative to wild type standardized at 100%. Such an enrichment is not the result of a natural response of a wild-type plant. Alternatively, or additionally, the isolated polypeptide is separated from other cellular components with which it is typically associated, e.g., by any of the various protein purification methods herein.

“Portion”, as used herein, refers to any part of a polynucleotide or polypeptide used for any purpose. This includes portions of polypeptides used in the screening of a library of molecules that specifically bind to a portion of a polypeptide or for the production of antibodies.

“Protein” refers to an amino acid sequence, oligopeptide, peptide, polypeptide or portions thereof whether naturally occurring or synthetic.

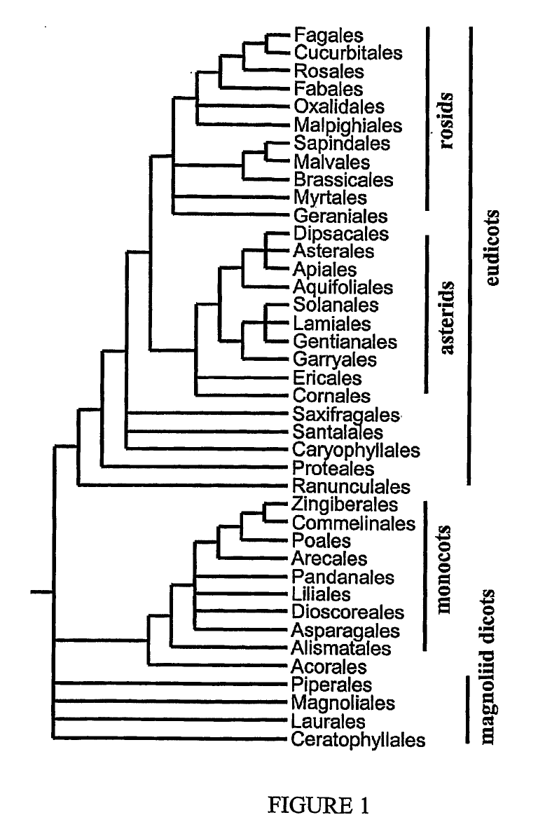

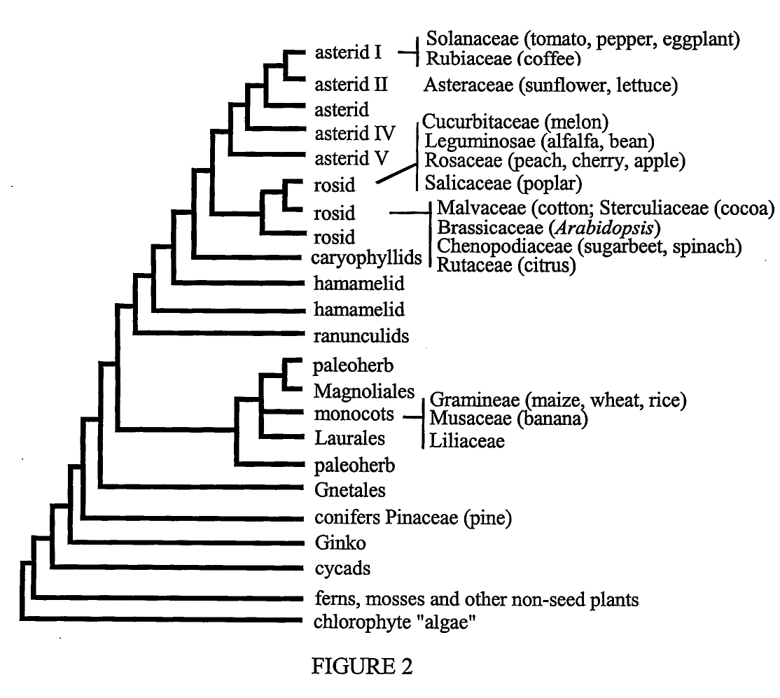

The term “plant” includes whole plants, shoot vegetative organs/structures (for example, leaves, stems and tubers), roots, flowers and floral organs/structures (for example, bracts, sepals, petals, stamens, carpels, anthers and ovules), seed (including embryo, endosperm, and seed coat) and fruit (the mature ovary), plant tissue (for example, vascular tissue, ground tissue, and the like) and cells (for example, guard cells, egg cells, and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, horsetails, psilophytes, lycophytes, bryophytes, and multicellular algae. (See for example, FIG. 1, adapted from Daly et al. (2001) Plant Physiol. 127: 1328-1333; FIG. 2, adapted from Ku et al. (2000) Proc. Natl. Acad. Sci. USA 97: 9121-9126; and see also Tudge, in The Variety of Life, Oxford University Press, New York, N.Y. (2000) pp. 547-606).

A “transgenic plant” refers to a plant that contains genetic material not found in a wild-type plant of the same species, variety or cultivar. The genetic material may include a transgene, an insertional mutagenesis event (such as by transposon or T-DNA insertional mutagenesis), an activation tagging sequence, a mutated sequence, a homologous recombination event or a sequence modified by chimeraplasty. Typically, the foreign genetic material has been introduced into the plant by human manipulation, but any method can be used as one of skill in the art recognizes.

A transgenic plant may contain an expression vector or cassette. The expression cassette typically comprises a polypeptide-encoding sequence operably linked (i.e., under regulatory control of) to appropriate inducible or constitutive regulatory sequences that allow for the expression of polypeptide.

The expression cassette can be introduced into a plant by transformation or by breeding after transformation of a parent plant. A plant refers to a whole plant, including seedlings and mature plants, as well as to a plant part, such as seed, fruit, leaf, or root, plant tissue, plant cells or any other plant material, e.g., a plant explant, as well as to progeny thereof, and to in vitro systems that mimic biochemical or cellular components or processes in a cell.

“Substrate” refers to any rigid or semi-rigid support to which nucleic acid molecules or proteins are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

A “trait” refers to a physiological, morphological, biochemical, or physical characteristic of a plant or particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring uptake of carbon dioxide, or by the observation of the expression level of a gene or genes, e.g., by employing Northern analysis, RT-PCR, microarray gene expression assays, or reporter gene expression systems, or by agricultural observations such as stress tolerance, yield, or pathogen tolerance. Any technique can be used to measure the amount of, comparative level of, or difference in any selected chemical compound or macromolecule in the transgenic plants, however.

“Trait modification” refers to a detectable difference in a characteristic in a plant ectopically expressing a polynucleotide or polypeptide of the present invention relative to a plant not doing so, such as a wild-type plant. In some cases, the trait modification can be evaluated quantitatively. For example, the trait modification can entail at least about a 2% increase or decrease in an observed trait (difference), at least a 5% difference, at least about a 10% difference, at least about a 20% difference, at least about a 30%, at least about a 50%, at least about a 70%, or at least about a 100%, or an even greater difference compared with a wild-type plant. It is known that there can be a natural variation in the modified trait. Therefore, the trait modification observed entails a change of the normal distribution of the trait in the plants compared with the distribution observed in wild-type plant.

“Transcript profile” refers to the expression levels of a set of genes in a cell in a particular state, particularly by comparison with the expression levels of that same set of genes in a cell of the same type in a reference state. The transcript profile of a particular transcription factor in a suspension cell corresponds to the expression levels of a set of genes in a cell overexpressing that transcription factor, compared with the expression levels of that same set of genes in a suspension cell that has normal levels of that transcription factor. The transcript profile can be presented as a list of those genes whose expression level is significantly different between the two treatments, and the difference ratios. Differences and similarities between expression levels may be evaluated and calculated using statistical and clustering methods.

“Wild type” or “wild-type”, as used herein, refers to a plant cell, seed, plant component, plant tissue, plant organ or whole plant that has not been genetically modified or treated in an experimental sense. Wild-type cells, seed, components, tissue, organs or whole plants may be used as controls to compare levels of expression and the extent and nature of trait modification with cells, tissue or plants of the same species in which a transcription factor expression is altered, e.g., in that it has been knocked out, overexpressed, or ectopically expressed.

A “control plant” as used herein refers to a plant cell, seed, plant component, plant tissue, plant organ or whole plant used to compare against transgenic or genetically modified plant for the purpose of identifying an enhanced phenotype in the transgenic or genetically modified plant. A control plant may in some cases be a transgenic plant line that comprises an empty vector or marker gene, but does not contain the recombinant polynucleotide of the present invention that is expressed in the transgenic or genetically modified plant being evaluated. In general, a control plant is a plant of the same line or variety as the transgenic or genetically modified plant being tested. A suitable control plant would include a genetically unaltered or non-transgenic plant of the parental line used to generate a transgenic plant herein.

Polypeptides and Polynucleotides of the Invention

The present invention provides, among other things, transcription factors, and transcription factor homolog polypeptides, and isolated or recombinant polynucleotides encoding the polypeptides, or novel sequence variant polypeptides or polynucleotides encoding novel variants of transcription factors derived from the specific sequences provided in the Sequence Listing. Also provided are methods for increasing a plant's tolerance to one or more pathogens or abiotic stresses. These methods are based on the ability to alter the expression of critical regulatory molecules that may be conserved between diverse plant species.

Related conserved regulatory molecules may be originally discovered in a model system such as Arabidopsis and homologous, functional molecules then discovered in other plant species. The latter may then be used to confer tolerance to one or more pathogens or abiotic stresses in diverse plant species.

Exemplary polynucleotides encoding the polypeptides of the invention were identified in the Arabidopsis thaliana GenBank database using publicly available sequence analysis programs and parameters. Sequences initially identified were then further characterized to identify sequences comprising specified sequence strings corresponding to sequence motifs present in families of known transcription factors. In addition, further exemplary polynucleotides encoding the polypeptides of the invention were identified in the plant GenBank database using publicly available sequence analysis programs and parameters. Sequences initially identified were then further characterized to identify sequences comprising specified sequence strings corresponding to sequence motifs present in families of known transcription factors. Polynucleotide sequences meeting such criteria were confirmed as transcription factors.

Additional polynucleotides of the invention were identified by screening Arabidopsis thaliana and/or other plant cDNA libraries with probes corresponding to known transcription factors under low stringency hybridization conditions. Additional sequences, including fall length coding sequences were subsequently recovered by the rapid amplification of cDNA ends (RACE) procedure, using a commercially available kit according to the manufacturer's instructions. Where necessary, multiple rounds of RACE are performed to isolate 5′ and 3′ ends. The full-length cDNA was then recovered by a routine end-to-end polymerase chain reaction (PCR) using primers specific to the isolated 5′ and 3′ ends. Exemplary sequences are provided in the Sequence Listing.

These sequences and others derived from diverse species and found in the Sequence Listing have been ectopically expressed in overexpressor or knockout plants. The changes in the characteristic(s) or trait(s) of the plants were then observed and found to confer increased abiotic stress or disease tolerance. Therefore, the polynucleotides and polypeptides can be used to improve desirable characteristics of plants.

The polynucleotides of the invention were also ectopically expressed in overexpressor plant cells and the changes in the expression levels of a number of genes, polynucleotides, and/or proteins of the plant cells observed. Therefore, the polynucleotides and polypeptides can be used to change expression levels of a genes, polynucleotides, and/or proteins of plants.

The AP2 family. AP2 (APETALA2) and EREBPs (Ethylene-Responsive Element Binding Proteins) are the prototypic members of a family of transcription factors unique to plants, whose distinguishing characteristic is that they contain the so-called AP2 DNA-binding domain (Riechmann and Meyerowitz (1998) Biol. Chem. 379: 633-646). The AP2 domain was first recognized as a repeated motif within the Arabidopsis thaliana AP2 protein (Jofuku et al. (1994) Plant Cell 6: 1211-1225). Four DNA-binding proteins from tobacco were identified that interact with a sequence that is essential for the responsiveness of some promoters to the plant hormone ethylene, and were designated as ethylene-responsive element binding proteins (EREBPs; Ohme-Takagi et al. (1995) supra). The DNA-binding domain of EREBP-2 was mapped to a region that was common to all four proteins (Ohme-Takagi et al (1995) supra), and that was found to be closely related to the AP2 domain (Weigel (1995) Plant Cell 7: 388-389) but that did not bear sequence similarity to previously known DNA-binding motifs.

AP2/EREBP genes form a large family, with many members known in several plant species (Okamuro et al. (1997) Proc. Natl. Acad. Sci. USA 94: 7076-7081; Riechmann and Meyerowitz (1998) supra). The number of AP2/EREBP genes in the Arabidopsis thaliana genome is approximately 145 (Riechmann et al. (2000) Science 290: 2105-2110). The APETALA2 class contains 14 genes and is characterized by the presence of two AP2 DNA binding domains. The AP2/ERF is the largest subfamily, and includes 125 genes that are involved in abiotic (DREB subgroup) and biotic (ERF subgroup) stress responses and the RAV subgroup includes six genes that all have a B3 DNA binding domain in addition to the AP2 DNA binding domain (Kagaya et al. (1999) Nucleic Acids Res. 27: 470-478).

Arabidopsis AP2 is involved in the specification of sepal and petal identity through its activity as a homeotic gene that forms part of the combinatorial genetic mechanism of floral organ identity determination, and it is also required for normal ovule and seed development (Bowman et al. (1991) Development 112: 1-20; Jofuku et al. (1994) supra). Arabidopsis ANT is required for ovule development and it also plays a role in floral organ growth (Elliott et al. (1996) Plant Cell 8: 155-168; Klucher et al. (1996) Plant Cell 8: 137-153). Finally, maize G115 regulates leaf epidermal cell identity (Moose et al. (1996) Genes Dev. 10: 3018-3027).

The attack of a plant by a pathogen may induce defense responses that lead to resistance to the invasion, and these responses are associated with transcriptional activation of defense-related genes, among them those encoding pathogenesis-related (PR) proteins. The involvement of EREBP-like genes in controlling the plant defense response is based on the observation that many PR gene promoters contain a short cis-acting element that mediates their responsiveness to ethylene (ethylene appears to be one of several signal molecules controlling the activation of defense responses). Tobacco EREBP-1, -2, -3, and -4, and tomato Pti4, Pti5 and Pti6 proteins have been shown to recognize such cis-acting elements (Ohme-Takagi (1995) supra; Zhou et al. (1997) EMBO J. 16: 3207-3218). In addition, Pti4, Pti5, and Pti6 proteins have been shown to interact directly with Pto, a protein kinase that confers resistance against Pseudomonas syringae pv tomato (Zhou et al. (1997) supra). Plants are also challenged by adverse environmental conditions such as cold or drought, and EREBP-like proteins appear to be involved in the responses to these abiotic stresses as well. COR (for cold-regulated) gene expression is induced during cold acclimation, the process by which plants increase their resistance to freezing in response to low temperatures. The Arabidopsis EREBP-like gene CBF1 (Stockinger et al. (1997) Proc. Natl. Acad. Sci. USA 94: 1035-1040) is a regulator of the cold acclimation response, because ectopic expression of CBF1 in Arabidopsis transgenic plants induced COR gene expression in the absence of a cold stimulus, and the plant freezing tolerance was increased (Jaglo-Ottosen et al. (1998) Science 280: 104-106). Another Arabidopsis EREBP-like gene, AB14, is involved in abscisic acid (ABA) signal transduction, because abi4 mutants are insensitive to ABA (ABA is a plant hormone that regulates many agronomically important aspects of plant development; Finkelstein et al. (1998) Plant Cell 10: 1043-1054).

Novel AP2 transcription factor genes and binding motifs in Arabidopsis and other diverse species. G28 corresponds to AtERF1 (GenBank accession number AB008103; Fujimoto et al. (2000) supra). G28 appears as gene AT4g17500 in the annotated sequence of Arabidopsis chromosome 4 (AL161546.2).

AtERF1 has been shown to have GCC-box binding activity; some defense-related genes that are induced by ethylene were found to contain a short cis-acting element known as the GCC-box: AGCCGCC (Ohme-Takagi et al. (1995) supra; and Ohme-Takagi and Shinshi (1990) Plant Mol. Biol. 15: 941-946. Using transient assays in Arabidopsis leaves, ATERF1 was found to be able to act as a GCC-box sequence specific transactivator (Fujimoto et al. (2000) supra).

AtERF1 expression has been described to be induced by ethylene (two- to three-fold increase in AtERF1 transcript levels 12 hours after ethylene treatment; Fujimoto et al. (2000) supra). In the ein2 mutant, the expression of AtERF1 was not induced by ethylene, suggesting that the ethylene induction of AtERF1 is regulated under the ethylene signaling pathway (Fujimoto et al. (2000) supra). AtERF1 expression was also induced by wounding, but not by other abiotic stresses (such as cold, salinity, or drought; Fujimoto et al. (2000) supra).

AtERF-type transcription factors respond to abiotic stress. While ERF-type transcription factors are primarily recognized for responding to a variety of biotic stresses (such as pathogen infection), some ERFs have been characterized as being responsive to abiotic stress. Fujimoto et. al. (Fujimoto et. al. (2000) Plant Cell 12: 393404 have shown that AtERF1, AtERF2, AtERF3, AtERF4, and, AtERF5, corresponding to G28, G1006, G1005, G6 and G1004 respectively, can respond to various abiotic stresses, including cold, heat, drought, ABA, CHX, and wounding. Genes normally associated with the plant defense response (PR1, PR2, PR5, and peroxidases) have also been shown to be regulated by water stress (Zhu et. al. (1995) Plant Physiol. 108: 929-937; Ingram and Bartels (1996). Annu Rev. Plant Physiol. Plant Mol. Biol. 47:377-403) suggesting some overlap between the two responses. A target sequence for ERF-type transcription factors has been identified and extensively studied (Hao et al. (1998) J. Biol. Chem. 273: 26857-26861). This target sequence consists of AGCCGCC and has been found in the 5′ upstream regions of genes responding to disease and regulated by ERFs. However, several genes (ARSK1 and dehydrin) known to be induced by ABA, NaCl, cold and wounding, also possess a GCC box regulatory element in their 5′ upstream regions (Hwang and Goodman (1995) Plant J. 8: 37-43), suggesting that ERF-type transcription factors may regulate also regulate abiotic stress associated genes.

ERF-type transcription factors in other species. ERF-type transcription factors have been characterized in other species. Tsi1, a tobacco AtERF ortholog has been shown to be responsive to NaCl, drought, wounding, salicylic acid (SA), ethephon, ABA, and methyl jasmonate (MeJA; Park et. al. (2001) Plant Cell 13: 1035-1046). Tsi1 is closely related to At4g27950 (G1750) in Arabidopsis. RT data suggest that G1750 may also have a similar function, although overexpression of G1750 causes some deleterious effects. In tobacco plants, however, overexpression of Tsi1 enhances resistance to both pathogen challenge and osmotic stress (Park et. al. (2001) supra). Interestingly, Tsi1 has also been shown to interact specifically with both GCC and DRE regulatory elements. Genes containing DRE elements are known to be regulated in response to abiotic stresses; as such, it is possible that Tsi1 has the ability to regulate the transcription of genes involved in abiotic stresses such as drought.

ERF-type transcription factors are well known to be transcriptional activators of disease responses (Fujimoto et. al. (2000) supra; Gu et al. (2000) Plant Cell 12: 771-786; Chen et al. (2002) Plant Cell 14: 559-574; Cheong et al. (2002) Plant Physiol. 129: 661-677; Onate-Sanchez and Singh (2002) Plant Physiol. 128: 1313-1322; Brown et al. (2003) Plant Physiol. 132: 1020-1032; Lorenzo et al. (2003) Plant Cell 15: 165-178) but have not been well characterized as being involved in response to abiotic stress conditions such as drought. Another group of AP2 transcription factors (DREBs), which includes the CBF class, are known to bind DRE elements in genes responding to abiotic stresses such as drought, high salt, and cold (Haake et al. (2002) Plant Physiol. 130: 639-648; Thomashow (2001) Plant Physiol. 125: 89-93, Liu et al. (1998) Plant Cell 10: 1391-1406; Gilmour et al. (2000) Plant Physiol. 124: 1854-1865; and Shinozaki and Yamaguchi-Shinozaki (2000) Curr. Opin. Plant Biol. 3: 217-223). However, there is growing evidence that ERF-type transcription factors can interact with not only the GCC-box, but also with regulatory elements present in genes that are responsive to osmotic stresses. Thus, it is becoming apparent from our studies as well as those of others that some ERF-type transcription factors may play a role in response to drought-related stress.

The role of ERF-type transcription factors in disease responses. The first indication that members of the ERF group might be involved in regulation of plant disease resistance pathways was the identification of Pti4, Pti5 and Pti6 as interactors with the tomato disease resistance protein Pto in yeast 2-hybrid assays (Zhou et al, (1997) EMBO J. 16: 3207-3218). Since that time, several ERF genes have been shown to enhance disease resistance when overexpressed in Arabidopsis or other species. These ERF genes include ERF1 (G1266) of Arabidopsis (Berrocal-Lobo et al. (2002) Plant J. 29: 23-32), Pti4 (Gu et al. (2002) Plant Cell 14: 817-831), and Pti5 (He et al. (2001) Mol. Plant Microbe Interact. 14: 1453-1457) of tomato, Tsi1 of tobacco (Park et. al. (2001) supra; Shin et al. (2002) Mol. Plant Microbe Interact. 15: 983-989), and AtERF1 (G28) and TDR1 (G1792) of Arabidopsis.

Regulation of ERF transcription factors by pathogen and small molecule signaling. ERF genes show a variety of stress-regulated expression patterns. Regulation by disease-related stimuli such as ethylene (ET), jasmonic acid (JA), SA, and infection by virulent or avirulent pathogens has been shown for a number of ERF genes (Fujimoto et. al. (2000) supra; Gu et al. (2000) supra; Chen et al. (2002) supra; Cheong et al. (2002) supra; Onate-Sanchez and Singh (2002) supra; Brown et al. (2003) supra; Lorenzo et al. (2003) supra). However, some ERF genes are also induced by wounding and abiotic stresses (Fujimoto et. al. (2000) supra; Park et al. (2001) supra; Chen et al. (2002) supra; Tournier et al. (2003) FEBS Lett. 550: 149-154). Currently, it is difficult to assess the overall picture of ERF regulation in relation to phylogeny, since different studies have concentrated on different ERF genes, treatments and time points. The advent of the Arabidopsis whole-genome microarray will result in more easily comparable data.

Significantly, several ERF transcription factors that confer enhanced disease resistance when overexpressed, such as ERF1, Pti4, and AtERF1, are transcriptionally regulated by pathogens, ET, and JA (Fujimoto et. al. (2000) supra; Onate-Sanchez and Singh (2002) supra; Brown et al. (2003) supra; Lorenzo et al. (2003) supra). ERF1 is induced synergistically by ET and JA, and induction by either hormone is dependent on an intact signal transduction pathway for both hormones, indicating that ERF1 may be a point of integration for ET and JA (Lorenzo et al. (2003) supra). At least four other ERFs are also induced by JA and ET (Brown et al. (2003) supra), implying that other ERFs are probably also important in ET/JA signal transduction. A number of the genes in subgroup 1, including AtERF3 and AtERF4, are thought to act as transcriptional repressors (Fujimoto et. al. (2000) supra), and these two genes were found to be induced by ET, JA, and an incompatible pathogen (Brown et al. (2003) supra). The net transcriptional effect on these pathways may be balanced between activation and repression of target genes.

The SA signal transduction pathway can act antagonistically to the ET/JA pathway. Interestingly, Pti4 and AtERF1 are induced by SA as well as by JA and ET (Gu et al. (2000) supra; Onate-Sanchez and Singh (2002) supra). Pti4, Pti5 and Pti6 have been implicated indirectly in regulation of the SA response, perhaps through interaction with other transcription factors, since overexpression of these genes in Arabidopsis induced SA-regulated genes without SA treatment and enhanced the induction seen after SA treatment (Gu et al. (2002) supra).

Post-transcriptional regulation of ERF genes by phosphorylation may be a significant form of regulation. Pti4 has been shown to be phosphorylated specifically by the Pto kinase, and this phosphorylation enhances binding to its target sequence (Gu et al. (2000) supra). Recently, the OsEREBP1 gene of rice has been shown to be phosphorylated by the pathogen-induced MAP kinase BWMK1, and this phosphorylation was shown to enhance its binding to the GCC box (Cheong et al. (2003) Plant Physiol. 132: 1961-1972), suggesting that phosphorylation of ERF proteins may be a common theme. A potential MAPK phosphorylation site has been noted in AtERF5 (Fujimoto et. al. (2000) supra).

Target genes regulated by ERF transcription factors. Binding of ERF transcription factors to the target sequence AGCCGCC (the GCC box) has been extensively studied (Hao et al. (1998) supra). This element is found in a number of promoters of pathogenesis-related and ET- or JA-induced genes. However, it is unclear how much overlap there is in target genes for particular ERFs. Recent studies have profiled genes induced in Arabidopsis plants overexpressing ERF1 (Lorenzo et al. (2003) supra) and Pti4 (Chakravarthy et al. (2003) Plant Cell 15: 3033-3050). However, these studies were done with different technology (Affymetrix GeneChip vs. serial analysis of gene expression) and under different conditions, and it is therefore difficult to compare the results directly. There is evidence that flanking sequences can affect the binding of ERFs to the GCC box (Gu et al. (2002) supra; Tournier et al. (2003) supra), so it is likely that different ERFs will regulate somewhat different gene sets. Direct comparisons of transcript profiles from plants overexpressing different ERFs, or of its vitro binding affinity of multiple ERFs to sites with varied flanking sequences, will likely be necessary to confirm conclusions about the degree of overlap in ERF target sets. Recent chromatin immunoprecipitation experiments with Pti4 suggest that it may also bind non-GCC box promoters, either directly or through interaction with other transcription factors (Chakravarthy et al. (2003) supra). This observation is particularly interesting in light of the hypothesis advanced by Gu et al. ((2002) supra) that Pti4 may regulate SA-induced genes through interaction with other transcription factors.

Identification of Residues and Motifs Unique to G28 Monocot Orthologs.

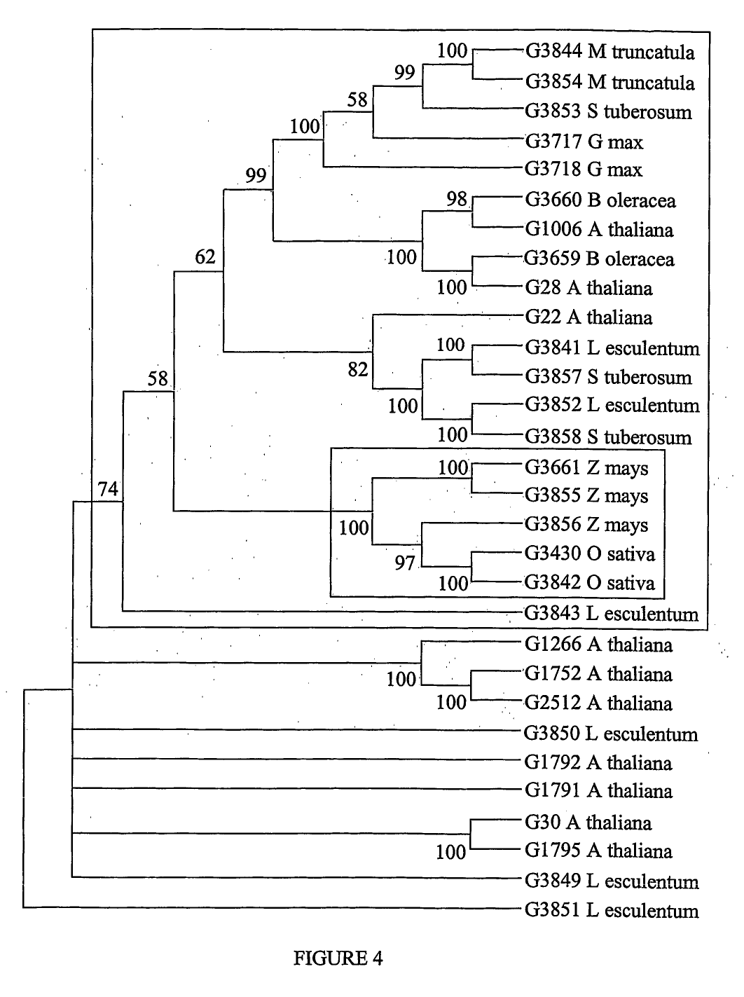

A number of sequences evolutionarily related to G28 were aligned using Clustal X (version 1.81, June 2000). Additional sequences were included in the alignment that were identified by BLASTP analysis of proprietary and public databases with protein sequences with a high degree of sequence relatedness to G28, particularly in the AP2 domain. A neighbor-joining algorithm comparing the AP2 domains of these sequences was then used to generate a phylogenetic tree, using Clustal X v1.81 's phylogenetic capabilities. Based on comparisons of the sequences in the alignment and, in particular, the phylogenetic analysis, the sequences with a common evolutionary history with reference to G28 were found in a separate lade, herein referred to as the “G28 clade of transcription factor polypeptides”, or simply the “G28 clade” (FIG. 4 provides an example of a phylogenetic tree that distinguishes the G28 lade from sequences outside of the lade).

Two sequences in this clade, G28 and a tomato sequence, Pti4, have been shown to confer enhanced disease tolerance when overexpressed in Arabidopsis (Heard (2004) U.S. Pat. No. 6,664,446; and Gu et al. (2002) Plant Cell 14, 817-831). One of the tobacco transcription factor genes has been shown previously to control the expression of basic PR genes, which are known to be involved in disease resistance responses (Kitajima et al. (2000) Plant Cell Physiol. 41: 817-824). Real time PCR experiments have shown that G28 and orthologs in Brassica napus (canola; orthologs Bn bh594074, Bn bh454277), Zea mays (G3661) and Oryza sativa (G3430) were induced by the disease-related hormone treatments MeJA and SA in the plant species in which they are found, consistent with a role for these genes in disease resistance. These observations support the premise that G28 lade sequences have conserved function across monocot and dicot lineages, and that the G28 clade comprises a number of genes involved in the control of disease resistance genes and the regulation of disease resistance.

After the G28 lade was identified, re-examination of the alignment of the sequences of the G28 clade of transcription factor polypeptides indicated a high degree of conservation of the AP2 DNA binding domain in all members of the lade. This enabled the definition of those sequence elements that define, structurally, the protein sequences comprising the G28 clade. There is also a high degree of conservation in additional motifs in all members of the clade. For example, residues corresponding to positions 76-85 of G28 (designated Motif X, SEQ ID NO: 56):

N/D D/Y A/S/T D/E/Q M/I L/V/F/A V/L/I/Q Y/F/N

are highly conserved in all members of the clade. The rest of Motif X, corresponding to positions 86-91 in G28, is less conserved, but is found in all members of the clade with the exception G3430:

X X L/M X D/E A/G

Within the G28 clade, a further subclade can be seen that includes only monocot sequences, and which share a common evolutionary history since the last common ancestor of monocots and dicots. Alignment of these sequences enabled the definition of those sequence elements that define, structurally, the sequences of the monocot subclade of the G28 clade. These monocot sequences were very similar in their AP2 domains and were distinguished from the dicot sequences by the presence of a highly conserved structural element or motif found just before (nearer the N-terminus) of Motif X. This sequence, herein referred to as “Motif Y”, may be represented by SEQ ID NO: 55 found in G3430, and corresponding to positions 45-61 of G3430. Motif Y is generally found as the subsequence:

S F G/W S/I L V/A A D Q/M W S D/E/G S L P F R.

This latter motif, shown in the monocot-derived sequences appearing in Tables 1 and 2, is considered to comprise a conserved structural element involved in the function of these monocot proteins, and provides a sequence element that is useful in the identification of other monocot transcription factor genes capable of conferring disease resistance in plants.

The monocot sequences within the G28 clade thus form a subclade within the G28 clade, said subgroup herein referred to as the “G3430 subclade of transcription factor polypeptides”, or simply the “G3430 subclade”.

Relatedness and utilities of the polynucleotides and polynucleotides of the invention. Table 1 shows the polypeptides identified by polypeptide SEQ ID NO (first column); Gene ID (GID) No.; (second column); the species of plant from which the sequence is derived (third column); the amino acid coordinates of the AP2 domain of the sequence (fourth column); the AP2 domain subsequences of the respective polypeptides (fifth column); the percentage identity to the AP2 domain of G3430 (found within SEQ ID NO: 10; sixth column); for monocot-derived sequences, the subsequence that is similar to Motif Y (seventh column); and the identity in percentage terms of each Motif Y subsequence to the Motif Y of SEQ ID NO: 55. These polypeptide sequences have AP2 domains with 75% or greater identity to the AP2 domain of G3430. Motif Ys in monocots are also highly conserved, and share 82% or greater identity with SEQ ID NO: 55 in the sequences that have been examined (see also Table 2).

TABLE 1
Gene families and binding domains

| | | | | | % ID | | |
|---|---|---|---|---|---|---|---|
| | | | AP2 | | to AP2 | | % ID to |
| SEQ | | | Domains in | | domain | Motif Y | Motif Y, |
| ID | GID | | AA | | of | subsequence | SEQ ID |
| NO: | No. | Species | Coordinates | AP2 domain | G3430 | (in monocots) | NO: 55 |
| 10 | G3430 | Oryza | 109-173 | RGKHYRGVRQRPWG | 100% | SFGSLVADQ | 100% |
| | | sativa | | KFAAEIRDPAKNGAR | | WSESLPFR | |
| | | | | VWLGTFDSAEEAAVA | | | |
| | | | | YDRAAYRMRGSRALL | | | |
| | | | | NFPLRI | | | |
| 30 | G3864 | Triticum | 127-191 | RGKHFRGVRQRPWG | 96% | SFGSLVADQ | 100% |
| | | aestivum | | KFAAEIRDPAKNGAR | | WSESLPFR | |
| | | | | VWLGTFDSAEDAAVA | | | |
| | | | | YDRAAYRMRGSRALL | | | |
| | | | | NFPLRI | | | |
| 32 | G3865 | Triticum | 125-189 | RGKHFRGVRQRPWG | 96% | SFGSLVADQ | 100% |
| | | aestivum | | KFAAEIRDPAKNGAR | | WSESLPFR | |
| | | | | VWLGTFDSAEDAAVA | | | |
| | | | | YDRAAYRMRGSRALL | | | |
| | | | | NFPLRI | | | |
| 34 | G3856 | Zea mays | 140-204 | RGKHYRGVRQRPWG | 96% | SFGSLVADQ | 100% |
| | | | | KFAAEIRDPAKNGAR | | WSESLPFR | |
| | | | | VWLGTYDSAEDAAV | | | |
| | | | | AYDRAAYRMRGSRA | | | |
| | | | | LLNFPLRI | | | |
| 36 | G3848 | Oryza | 149-213 | RGKHYRGVRQRPWG | 95% | SFGSLVAD | 88% |
| | sativa | | | KFAAEIRDPAKNGAR | | MWSDSLPFR | |
| | | | | VWLGTFDTAEDAALA | | | |
| | | | | YDRAAYRMRGSRALL | | | |
| | | | | NFPLRI | | | |
| 12 | G3661 | Zea mays | 126-190 | RGKHYRGVRQRPWG | 92% | SFGSLVADQ | 94% |
| | | | | KFAAEIRDPARNGAR | | WSGSLPFR | |
| | | | | VWLGTYDTAEDAAL | | | |
| | | | | AYDRAAYRMRGSRA | | | |
| | | | | LLNFPLRI | | | |
| 26 | G3718 | Glycine | 139-203 | KGKHYRGVRQRPWG | 92% | | |
| | max | | | KFAAEIRDPAKNGAR | | | |
| | | | | VWLGTFETAEDAALA | | | |
| | | | | YDRAAYRMRGSRALL | | | |
| | | | | NFPLRI | | | |
| 8 | G3717 | Glycine | 130-194 | KGKHYRGVRQRPWG | 90% | | |
| | max | | | KFAAEIRDPAKNGAR | | | |
| | | | | VWLGTFETAEDAALA | | | |
| | | | | YDRAAYRMRGSRALL | | | |
| | | | | NFPLRV | | | |
| 24 | G3844 | Medicago | 141-205 | KGKHYRGVRQRPWG | 90% | | |
| | | truncatula | | KFAAEIRDPAKNGAR | | | |
| | | | | VWLGTFETAEDAALA | | | |
| | | | | YDRAAYRMRGSRALL | | | |
| | | | | NFPLRV | | | |
| 2 | G28 | Arabidopsis | 144-208 | KGKHYRGVRQRPWG | 89% | | |
| | | thaliana | | KFAAEIRDPAKNGAR | | | |
| | | | | VWLGTFETAEDAALA | | | |
| | | | | YDRAAFRMRGSRALL | | | |
| | | | | NFPLRV | | | |
| 20 | G3659 | Brassica | 130-194 | KGKHYRGVRQRPWG | 89% | | |
| | | oleracea | | KFAAEIRDPAKGAR | | | |
| | | | | VWLGTFETAEDAALA | | | |
| | | | | YDRAAFRMRGSRALL | | | |
| | | | | NFPLRV | | | |
| 4 | G1006 | Arabidopsis | 113-177 | KAKHYRGVRQRPWG | 86% | | |
| | | thaliana | | KFAAEIRDPAKNGAR | | | |
| | | | | VWLGTFETAEDAALA | | | |
| | | | | YDIAAFRMRGSRALL | | | |
| | | | | NFPLRV | | | |
| 22 | G3660 | Brassica | 119-183 | KGKHYRGVRQRPWG | 86% | | |
| | | oleracea | | KFAAEIRDPAKKGAR | | | |
| | | | | EWLGTFETAEDAALA | | | |
| | | | | YDRAAFRMRGSRALL | | | |
| | | | | NFPLRV | | | |
| 16 | G3846 | Nicotiana | 95-159 | KGRHYRGVRQRPWG | 86% | | |
| | | tabacum | | KFAAEIRDPAKNGAR | | | |
| | | | | VWLGTYETAEEAALA | | | |
| | | | | YDKAAYRMRGSKAL | | | |
| | | | | LNFPHRI | | | |
| 28 | G3843 | Lycopersicon | 130-194 | KAKHYRGVRVRPWG | 84% | | |
| | | esculentum | | KFAAEIRDPAKNGAR | | | |
| | | | | VWLGTYETAEDAALA | | | |
| | | | | YDKAAFRMRGSRALL | | | |
| | | | | NFPLRI | | | |
| 18 | G3841 | Lycopersicon | 102-166 | KGRHYRGVRQRPWG | 84% | | |
| | Pti4 | esculentum | | KFAAEIRDPAKNGAR | | | |
| | | | | VWLGTYETAEEAAIA | | | |
| | | | | YDKAAYRMRGSKAH | | | |
| | | | | LNFPHRI | | | |
| 42 | G3858 | Solanum | 108-172 | KGRHYRGVRQRPWG | 84% | | |
| | | tuberosum | | KFAAEIRDPAKNGAR | | | |
| | | | | VWLGTYESAEEAALA | | | |
| | | | | YDIAAFRMRGTKALL | | | |
| | | | | NFPHRI | | | |
| 38 | G3857 | Solanum | 98-162 | KGRHYRGVRQRPWG | 84% | | |
| | | tuberosum | | KFAAEIRDPAKNGAR | | | |
| | | | | VWLGTYETAEEAAIA | | | |
| | | | | YDKAAYRMRGSKAH | | | |
| | | | | LNFPHRI | | | |
| 40 | G3852 | Lycopersicon | 103-167 | KGRHYRGVRQRPWG | 83% | | |
| | | esculentum | | KFAAEIRDPAKNGAR | | | |
| | | | | VWLGTYESAEEAALA | | | |
| | | | | YGKAAFRMRGTKALL | | | |
| | | | | NFPHRI | | | |
| 14 | G3845 | Nicotiana | 101-165 | RGRHYRGVRRRPWG | 83% | | |
| | | tabacum | | KFAAEIRDPAKNGAR | | | |
| | | | | VWLGTYETDEEAAIA | | | |
| | | | | YDKAAYRMRGSKAH | | | |
| | | | | LNFPHRI | | | |
| 60 | G22 | Arabidopsis | 88-152 | KGMQYRGVRRRPWG | 75% | | |
| | | thaliana | | KFAAEIRDPKKNGAR | | | |
| | | | | VWLGTYETPEDAAVA | | | |
| | | | | YDRAAFQLRGSKAKL | | | |
| | | | | NFPHLI | | | |

The transcription factors of the invention each possess an AP2 domain, and include paralogs and orthologs of G28 and G3430 found by BLAST analysis, as described below. The transcription factors of the invention that are derived from monocot plants also contain a Motif Y.

TDR polypeptides share several potential protein kinase phosphorylation sites, in particular those phosphorylation sites in regions homologous to that of the Arabidopsis phosphorylation sites at amino acid residues S67, S100, S101, S102, S111, S220, S223, S224, S227 of SEQ ID NO: 2 (G28) and at amino acid residues S73, T188, S189, S192, S193, S194, S204 of SEQ ID NO: 4 (G1006). The potential protein kinase phosphorylation sites are sites that may be modified by a protein kinase selected from, but not limited to, an isoform of protein kinase C, protein kinase A, protein kinase G, casein kinase II, or Pto kinase.

Eleven TDR polypeptide sequences share at least three conserved regions distinct from the AP2 domain. One region, amino acid consensus sequence 1 motif, is exemplified by contiguous amino acid residues L71 through F91 of SEQ ID NO: 2 and has the consensus sequence Leu-Pro-Leu/Phe-Lys/Arg-Glu/Pro/hrSer/Gly/Asp-Asn/Asp-Asp-Ser/Ala-Glu/Asp-Asp-Met-Leu-Val-Val/Leu/Ile-Tyr/Phe-Gly/Thr-Ile/Leu/Val/Ala-Leu-Xaa-Asp-Ala-Phe/Leu/Val, where Xaa is any amino acid residue. A second region, amino acid consensus sequence 2 motif, is exemplified by contiguous amino acid residues K235 through R238 of SEQ ID NO: 2, and comprises basic residues with the consensus sequence Lys-Lys/Arg-Arg/Lys-Arg/Lys. A third region, amino acid consensus sequence 3 motif, is exemplified by contiguous amino acid residues G262 through L268 of SEQ ID NO: 2, and has the consensus sequence Gly/Val/Arg-Asp/Glu/His-Arg/Glu/Gln-Leu-Leu/Val-Val. A fourth region, exemplified by contiguous amino acid residues P213 through R238 of SEQ ID NO: 2, has at least one phosphorylation site flanked by the consensus sequences Pro-Asp/Glu-Pro and Lys-Lys/Arg-Arg/Lys-Lys/Arg and the phosphorylation site is potentially phosphorylated by at least one isozyme of protein kinase C, protein kinase A, protein kinase G, casein kinase II, or Pto kinase.

The AP2 domains of eleven TDR polypeptide sequences comprise a consensus sequence of Gly-Lys-His-Tyr-Arg-Gly-Val-Arg-Gln/Arg-Arg-Pro-Trp-Gly-Lys/Glu-Phe-Ala-Ala-Glu-Ile-Arg-Asp-Pro-Ala-Lys/Arg-Asn-Gly-Ala-Arg-Val-Trp-Leu/His-Gly-Thr-Phe/Tyr-Asp/Glu-Thr/Ser-Ala/Asp-Glu-Asp/Glu-Ala-Ala-Leu/Val/Ile-Ala-Tyr-Asp-Arg/Lys/Re-Ala-Ala-Phe/Tyr-Arg-Met/Arg-Arg-Gly-ser-Arg/Lys-Ala-Leu/His-Leu-Asn-Phe-Pro-Leu/His-Arg-Val/Ile-Asn/Gly-Ser/Leu-Gly/lu/Asn-Glu/Asp/Ile-Pro.

The G28 lade is distinguished by, for example, an AP2 domain, an arginine residue at a position corresponding to position 222 of SEQ ID NO: 2, and the ability to confer disease tolerance or resistance in plants. In this context, “corresponding position” refers to a similar or the same position in an alignment of two similar or identical subsequences of distinct G28 lade polypeptides. The sequences that appear in an alignment of polypeptides such as that found in FIGS. 3A-3G (for the present discussion, R222 of G28 and residues in the same clade and column in FIG. 3D) may be used to determine corresponding residues. It will be recognized by those skilled in the art that similar substitutions, such as those identified in Table 5, may be made to corresponding residues in polypeptides that retain the function of the unsubstituted molecule.

The G3430 subclade of the G28 clade of transcription factors includes the monocot-derived sequences within the G28 lade. The G3430 subclade may be distinguished by the presence of a Motif Y, a 17 amino acid residue that is substantially identical to SEQ ID NO: 55.

Therefore, the invention provides tdr polynucleotides comprising SEQ ID NO: 1, paralogs, orthologs, and/or equivalog sequences and encoding TDR polypeptides that are members of the G28 lade of transcription factor polypeptides. The polynucleotides are shown to have strong differential expression associated with response to plant pathogen exposure. The invention also encompasses a complement of the polynucleotides. The polynucleotides are useful for screening libraries of molecules or compounds for specific binding and for creating transgenic plants having increased tolerance to pathogens.

Additional polynucleotides of the invention were identified by screening Arabidopsis thaliana and/or other plant cDNA libraries with probes corresponding to known transcription factors under low stringency hybridization conditions. Additional sequences, including full length coding sequences, were subsequently recovered by the rapid amplification of cDNA ends (RACE) procedure, using a commercially available kit according to the manufacturer's instructions. Where necessary, multiple rounds of RACE were performed to isolate 5′ and 3′ ends. The full-length cDNA was then recovered by a routine end-to-end polymerase chain reaction (PCR) using primers specific to the isolated 5′ and 3′ ends. Exemplary sequences are provided in the Sequence Listing.

The polynucleotides are particularly useful when they are hybridizable array elements in a microarray. Such a microarray can be employed to monitor the expression of genes that are differentially expressed in normal, diseased, or callous tissues. The microarray can be used in large scale genetic or gene expression analysis of a large number of polynucleotides; in the diagnosis of plant diseases or disorders before phenotypic symptoms are evident. Furthermore, the microarray can be employed to investigate cellular responses, such as cell proliferation, transformation, and the like. The array elements may be organized in an ordered fashion so that each element is present at a specified location on the substrate. Because the array elements are at specified locations on the substrate, the hybridization patterns and intensities (that together create a unique expression profile) can be interpreted in terms of expression levels of particular genes and can be correlated with a particular disease, pathology, or treatment.

The invention also entails an agronomic composition comprising a polynucleotide of the invention in conjunction with a suitable carrier and a method for altering a plants trait using the composition.

The invention also encompasses transcription factor polypeptides that comprise SEQ ID NO: 55, or a motif that is substantially identical to SEQ ID NO: 55, and have substantially similar activity with that of SEQ ID NO: 2. For example, SEQ ID NO: 10 and SEQ ID NO: 12 include the subsequence:

Ser Phe Gly Ser Leu Val Ala Asp Gln Trp Ser Xaa Ser Leu Pro Phe Arg

where Xaa represents any naturally occurring amino acid residue.

Transcription factor polypeptides that comprise SEQ ID NO: 55 or a motif that is substantially identical to SEQ ID NO: 55, and that have substantially similar functions as G28 or G3430 in conferring disease tolerance or resistance in plants when overexpressed, are intended to fall within the scope of the invention.

Additional monocot ortholog sequences identified using conservation to motif Y. As a conserved motif found in two monocot orthologs of SEQ ID NO: 2, motif Y was used to identify additional monocot orthologs of SEQ ID G28. Motif Y was used in a TBLASTN search against all plant nucleotide sequences in GenBank. A significant number of monocot sequences were found that had a minimum of 14 identical residues to the 17 residue Motif Y of SEQ ID NO: 55 (Table 2). Monocot sequences were the only sequences found in this analysis; no dicot Motif Y-like sequences were identified, even allowing for three mismatches to SEQ ID NO: 55. Upon translation of these nucleotide sequences in a frame that provided the identified conserved motif, all the resulting protein sequences were found to have a conserved AP2 binding domain in the expected location. The protein sequences having a conserved AP2 binding domain in the expected location were aligned with the previously aligned set of AP2 sequences, and a neighbor-joining algorithm was used to generate a phylogenetic tree, as described above. In this tree, the additional sequences identified through Motif Y all were found within the G28 clade identified previously, indicating that Motif Y was successfully used to identify new monocot orthologs of G28, listed in Table 2.

TABLE 2
Published Sequences that Comprise Subsequences Highly Similar to Motif Y, SEQ ID NO: 55

| | | | Percent Identity |
|---|---|---|---|
| GenBank | | | to SEQ ID NO: |
| Accession No. | Species | Motif Y Sequence | 55 |
| AU057740 | Oryza sativa | SFGSLVADQWSESLPFR | 100% |
| AX573798 | Oryza sativa | SFGSLVADQWSESLPFR | 100% |
| AX653155 | Oryza sativa | SFGSLVADQWSESLPFR | 100% |
| AK105940 | Oryza sativa | SFGSLVADQWSESLPFR | 100% |
| AK073812 | Oryza sativa | SFGSLVADQWSESLPFR | 100% |
| AJ307662 | Oryza sativa | SFGSLVADQWSESLPFR | 100% |
| CB653231 | Oryza sativa | SFGSLVADQWSESLPFR | 100% |
| AP004676 | Oryza sativa (japonica cultivar-group) | SFGSLVADQWSESLPFR | 100% |
| AAAA01012531 | Oryza sativa (indica cultivar-group) | SFGSLVADQWSESLPFR | 100% |
| CL163362 | Sorghum bicolor | SFGSLVADQWSESLPFR | 100% |
| CD211509 | Sorghum bicolor | SFGSLVADQWSESLPFR | 100% |
| CN130468 | Sorghum bicolor | SFGSLVADQWSESLPFR | 100% |
| BF705208 | Sorghum propinquum | SFGSLVADQWSESLPFR | 100% |
| AL821943 | Triticum aestivum | SFGSLVADQWSESLPFR | 100% |
| CK195316 | Triticum aestivum | SFGSLVADQWSESLPFR | 100% |
| CN012725 | Triticum aestivum | SFGSLVADQWSESLPFR | 100% |
| CN011872 | Triticum aestivum | SFGSLVADQWSESLPFR | 100% |
| CN010562 | Triticum aestivum | SFGSLVADQWSESLPFR | 100% |
| CA741180 | Triticum aestivum | SFGSLVADQWSESLPFR | 100% |
| BE427897 | Triticum turgidum subsp. Durum | SFGSLVADQWSESLPFR | 100% |
| CA004558 | Hordeum vulgare subsp. vulgare | SFGSLVADQWSESLPFR | 100% |
| BQ467769 | Hordeum vulgare subsp. vulgare | SFGSLVADQWSESLPFR | 100% |
| CG333070 | Zea mays | SFGSLVADQWSESLPFR | 100% |
| CF626193 | Zea mays | SFGSLVADQWSESLPFR | 100% |
| CG355473 | Zea mays | SFGSLVADQWSESLPFR | 100% |
| CC702573 | Zea mays | SFGSLVADQWSESLPFR | 100% |
| CA121404 | Saccharum officinarum | SFGSLVADQWSGSLPFR | 94% |
| CA141374 | Saccharum officinarum | SFGSLVADQWSGSLPFR | 94% |
| BQ537427 | Saccharum officinarum | SFGSLVADQWSGSLPFR | 94% |
| CA121403 | Saccharum officinarum | SFGSLVADQWSGSLPFR | 94% |
| AW680814 | Sorghum bicolor | SFGSLVADQWSGSLPFR | 94% |
| BG357344 | Sorghum bicolor | SFGSLVADQWSGSLPFR | 94% |
| BG948711 | Sorghum bicolor | SFGSLVADQWSGSLPFR | 94% |
| CG283767 | Zea mays | SFGSLVADQWSGSLPFR | 94% |
| CG239914 | Zea mays | SFGSLVADQWSGSLPFR | 94% |
| CB661210 | Oryza sativa | SFGSLVADMWSDSLPFR | 88% |
| CB670319 | Oryza sativa | SFGSLVADMWSDSLPFR | 88% |
| CB670372 | Oryza sativa | SFGSLVADMWSDSLPFR | 88% |
| CB641135 | Oryza sativa | SFGSLVADMWSDSLPFR | 88% |
| AL607006 | Oryza sativa (japonica cultivar-group) | SFGSLVADMWSDSLPFR | 88% |
| AU197778 | Oryza sativa (japonica cultivar-group) | SFGSLVADMWSDSLPFR | 88% |
| AX654311 | Oryza sativa | SFGSLVADMWSDSLPFR | 88% |
| CB666299 | Oryza sativa | SFGSLVADMWSDSLPFR | 88% |
| CB675534 | Oryza sativa | SFGSLVADMWSDSLPFR | 88% |
| CB660138 | Oryza sativa | SFGSLVADMWSDSLPFR | 88% |
| D23520 | Oryza sativa (japonica cultivar-group) | SFGSLVADMWSDSLPFR | 88% |
| AAAA01003158 | Oryza sativa (indica cultivar-group) | SFGSLVADMWSDSLPFR | 88% |
| C25163 | Oryza sativa (japonica cultivar-group) | SFGSLVADMWSXSLPFR | 88% |
| CN145823 | Sorghum bicolor | SFGSLAADQWSGSLPFR | 88% |
| CG261750 | Zea mays | SFGILVADQWSDSLPFR | 88% |
| CG230966 | Zea mays | SFGILVADQWSDSLPFR | 88% |
| CG230975 | Zea mays | SFGILVADQWSDSLPFR | 88% |
| CG233760 | Zea mays | SFGILVADQWSDSLPFR | 88% |
| CB673022 | Oryza sativa | SFWSLVADMWSDSLPFR | 82% |

The correlation between the conserved structural element Motif Y and disease resistance-conferring transcription factors in monocots is striking and, as determined thus far, absolute; Motif Y was always present in monocots nearer the N-terminus than the AP2 domain, but never found in dicots. Motif Y is associated with transcription factors that are part of a lade of AP2 transcription factors known to confer disease resistance, and is thus highly likely to be involved in the disease resistance function of these transcription factors in monocots. Table 2, which shows a number of sequences found to contain a Motif Y, includes sequences discovered in cDNA libraries from wheat plants challenged with Fusarium graminearum (Kruger et al. (2004) NCBI accession numbers CN011872, CN010562 and CN012725). These libraries contained genes of both fungal and plant origin. The authors of these reports appear to have discovered, without identifying a specific function, AP2 transcription factors that contain a Motif Y. The function of these sequences that are apparently produced during fungal challenge is likely attributable to an inducible disease tolerance mechanism. Because of the correlation of Motif Y and disease tolerance-associated transcription factors in monocots, Motif Y is likely to be required for, or to enhance, the up-regulation of pathways involved in conferring disease tolerance or resistance in monocots, a hypothesis that may readily be tested for each monocot plant in which Motif Y is found.

Producing Polypeptides. The polynucleotides of the invention include sequences that encode transcription factors and transcription factor homolog polypeptides and sequences complementary thereto, as well as unique fragments of coding sequence, or sequence complementary thereto. Such polynucleotides can be, e.g., DNA or RNA, e.g., mRNA, cRNA, synthetic RNA, genomic DNA, cDNA synthetic DNA, oligonucleotides, etc. The polynucleotides are either double-stranded or single-stranded, and include either, or both sense (i.e., coding) sequences and antisense (i.e., non-coding, complementary) sequences. The polynucleotides include the coding sequence of a transcription factor, or transcription factor homolog polypeptide, in isolation, in combination with additional coding sequences (e.g., a purification tag, a localization signal, as a fusion-protein, as a pre-protein, or the like), in combination with non-coding sequences (e.g., introns or inteins, regulatory elements such as promoters, enhancers, terminators, and the like), and/or in a vector or host environment in which the polynucleotide encoding a transcription factor or transcription factor homolog polypeptide is an endogenous or exogenous gene.

A variety of methods exist for producing the polynucleotides of the invention. Procedures for identifying and isolating DNA clones are well known to those of skill in the art, and are described in, e.g., Berger and Kimmel (1987) Guide to Molecular Cloning Techniques, Methods in Enzymology, vol. 152 Academic Press, Inc., San Diego, Calif.; Sambrook et al. (1989) supra, and Ausubel et al. editors, (supplemented through 2000) Current Protocols in Molecular Biology, Current Protocols, a joint venture between Greene Publishing Associates, Inc. and John Wiley & Sons, Inc.

Alternatively, polynucleotides of the invention, can be produced by a variety of in vitro amplification methods adapted to the present invention by appropriate selection of specific or degenerate primers. Examples of protocols sufficient to direct persons of skill through in vitro amplification methods, including the polymerase chain reaction (PCR) the ligase chain reaction (LCR), Qβ-replicase amplification and other RNA polymerase mediated techniques (e.g., NASBA), e.g., for the production of the homologous nucleic acids of the invention are found in Berger (1987) supra, Sambrook et al. (1989) supra), and Ausubel (2000) supra), as well as Mullis et al. (1990) PCR Protocols A Guide to Methods and Applications (Innis et al. eds) Academic Press Inc. San Diego, Calif. Improved methods for cloning in vitro amplified nucleic acids are described in Wallace et al. U.S. Pat. No. 5,426,039. Improved methods for amplifying large nucleic acids by PCR are summarized in Cheng et al. (1994) Nature 369: 684-685 and the references cited therein, in which PCR amplicons of up to 40 kb are generated. One of skill will appreciate that essentially any RNA can be converted into a double stranded DNA suitable for restriction digestion, PCR expansion and sequencing using reverse transcriptase and a polymerase. See, e.g., Ausubel (2000) supra, Sambrook et al. (1989) supra, and Berger (1987) supra.

Alternatively, polynucleotides and oligonucleotides of the invention can be assembled from fragments produced by solid-phase synthesis methods. Typically, fragments of up to approximately 100 bases are individually synthesized and then enzymatically or chemically ligated to produce a desired sequence, e.g:, a polynucleotide encoding all or part of a transcription factor. For example, chemical synthesis using the phosphoramidite method is described (e.g., by Beaucage et al. (1981) Tetrahedron Letters 22: 1859-1869; and Matthes et al. (1984) EMBO J. 3: 801-805). According to such methods, oligonucleotides are synthesized, purified, annealed to their complementary strand, ligated and then optionally cloned into suitable vectors. And if so desired, the polynucleotides and polypeptides of the invention can be custom ordered from any of a number of commercial suppliers.

Homologous Sequences. Sequences homologous to those provided in the Sequence Listing, derived from Arabidopsis thaliana or from other plants of choice, are also an aspect of the invention. Homologous sequences can be derived from any plant including monocots and dicots and in particular agriculturally important plant species, including but not limited to, crops such as soybean, wheat, corn (maize), potato, cotton, rice, rape, oilseed rape (including canola), sunflower, alfalfa, clover, sugarcane, and turf; or fruits and vegetables, such as banana, blackberry, blueberry, strawberry, and raspberry, cantaloupe, carrot, cauliflower, coffee, cucumber, eggplant, grapes, honeydew, lettuce, mango, melon, onion, papaya, peas, peppers, pineapple, pumpkin, spinach, squash, sweet corn, tobacco, tomato, tomatillo, watermelon, rosaceous fruits (such as apple, peach, pear, cherry and plum) and vegetable brassicas (such as broccoli, cabbage, cauliflower, Brussels sprouts, and kohlrabi). Other crops, including fruits and vegetables, whose phenotype can be changed and that comprise homologous sequences include barley; rye; millet; sorghum; currant; avocado; citrus fruits such as oranges, lemons, grapefruit and tangerines, artichoke, cherries; nuts such as the walnut and peanut; endive; leek; roots such as arrowroot, beet, cassaya, turnip, radish, yam, and sweet potato; and beans. The homologous sequences may also be derived from woody species, such pine, poplar and eucalyptus, or mint or other labiates. In addition, homologous sequences may be derived from plants that are evolutionarily-related to crop plants, but which may not have yet been used as crop plants. Examples include deadly nightshade (Atropa belladona), related to tomato; jimson weed (Datura strommium), related to peyote; and teosinte (Zea species), related to corn (maize).

Orthologs and Paralogs. Homologous sequences as described above can comprise orthologous or paralogous sequences. Several different methods are known by those of skill in the art for identifying and defining these functionally homologous sequences. Three general methods for defining orthologs and paralogs are described. Orthologs, paralogs, or equivalogs may be identified by one or more of the methods described below.

Orthologs and paralogs are evolutionarily related genes that have similar sequence and similar functions. Orthologs are structurally related genes in different species that are derived by a speciation event. Paralogs are structurally related genes within a single species that are derived by a duplication event.

Within a single plant species, gene duplication may cause two copies of a particular gene, giving rise to two or more genes with similar sequence and often similar function known as paralogs. A paralog is therefore a similar gene formed by duplication within the same species. Paralogs typically cluster together or in the same clade (a group of similar genes) when a gene family phylogeny is analyzed using programs such as CLUSTAL (Thompson et al. (1994) Nucleic Acids Res. 22: 4673-4680; Higgins et al. (1996) Methods Enzymol. 266: 383-402). Groups of similar genes can also be identified with pair-wise BLAST analysis (Feng and Doolittle (1987) J. Mol. Evol. 25: 351-360). For example, a lade of very similar MADS domain transcription factors from Arabidopsis all share a common function in flowering time (Ratcliffe et al. (2001) Plant Physiol. 126: 122-132), and a group of very similar AP2 domain transcription factors from Arabidopsis are involved in tolerance of plants to freezing (Gilmour et al. (1998) Plant J. 16: 433-442). Analysis of groups of similar genes with similar function that fall within one lade can yield sub-sequences that are particular to the lade. These sub-sequences, known as consensus sequences, can not only be used to define the sequences within each lade, but define the functions of these genes; genes within a clade may contain paralogous sequences, or orthologous sequences that share the same function (see also, for example, Mount (2001), in Bioinformatics: Sequence and Genome Analysis, Cold Spring Harbor Laboratory Press, Cold Spring Harbor, N.Y., page 543).

Speciation, the appearance of new species from a parental species, can also give rise to two or more genes with similar sequence and similar function. These genes, termed orthologs, often have an identical function within their host plants and are often interchangeable between species without losing function. Because plants have common ancestors, many genes in any plant species will have a corresponding orthologous gene in another plant species. Once a phylogenic tree for a gene family of one species has been constructed using a program such as CLUSTAL (Thompson et al. (1994) Nucleic Acids Res. 22: 4673-4680; Higgins et al. (1996) supra) potential orthologous sequences can be placed into the phylogenetic tree and their relationship to genes from the species of interest can be determined. Orthologous sequences can also be identified by a reciprocal BLAST strategy. Once an orthologous sequence has been identified, the function of the ortholog can be deduced from the identified function of the reference sequence.

Transcription factor gene sequences are conserved across diverse eukaryotic species lines (Goodrich et al. (1993) Cell 75: 519-530; Lin et al. (1991) Nature 353: 569-571; Sadowski et al. (1988) Nature 335: 563-564). Plants are no exception to this observation; diverse plant species possess transcription factors that have similar sequences and functions.

Orthologous genes from different organisms have highly conserved functions, and very often essentially identical functions (Lee et al. (2002) Genome Res. 12: 493-502; Remm et al. (2001) J. Mol. Biol. 314: 1041-1052). Paralogous genes, which have diverged through gene duplication, may retain similar functions of the encoded proteins. In such cases, paralogs can be used interchangeably with respect to certain embodiments of the instant invention (for example, transgenic expression of a coding sequence). An example of such highly related paralogs is the CBF family, with three well-defined members in Arabidopsis and at least one ortholog in Brassica napus (SEQ ID NOs: 46, 48, 50, or 52, respectively), all of which control pathways involved in both freezing and drought stress (Gilmour et al. (1998) Plant J. 16: 433-442; Jaglo et al. (1998) Plant Physiol. 127: 910-917).

The following references represent a small sampling of the many studies that demonstrate that conserved transcription factor genes from diverse species are likely to function similarly (i.e., regulate similar target sequences and control the same traits), and that transcription factors may be transformed into diverse species to confer or improve traits.
(1) The Arabidopsis NPR1 gene regulates systemic acquired resistance (SAR); over-expression of NPR1 leads to enhanced resistance in Arabidopsis. When either Arabidopsis NPR1 or the rice NPR1 ortholog was overexpressed in rice (which, as a monocot, is diverse from Arabidopsis), challenge with the rice bacterial blight pathogen Xanthomonas oryzae pv. oryzae, the transgenic plants displayed enhanced resistance (Chem et al. (2001) Plant J 27: 101-113). NPR1 acts through activation of expression of transcription factor genes, such as TGA2 (Fan and Dong (2002) Plant Cell 14: 1377-1389).
(2) E2F genes are involved in transcription of plant genes for proliferating cell nuclear antigen (PCNA). Plant E2Fs share a high degree of similarity in amino acid sequence between monocots and dicots, and are even similar to the conserved domains of the animal E2Fs. Such conservation indicates a functional similarity between plant and animal E2Fs. E2F transcription factors that regulate meristem development act through common cis-elements, and regulate related (PCNA) genes (Kosugi and Ohashi, (2002) Plant J. 29: 45-59).
(3) The ABI5 gene (ABA Insensitive 5) encodes a basic leucine zipper factor required for ABA response in the seed and vegetative tissues. Co-transformation experiments with ABI5 cDNA constructs in rice protoplasts resulted in specific transactivation of the ABA-inducible wheat, Arabidopsis, bean, and barley promoters. These results demonstrate that sequentially similar ABI5 transcription factors are key targets of a conserved ABA signaling pathway in diverse plants (Gampala et al. (2001) J. Biol. Chem. 277: 1689-1694).
(4) Sequences of three Arabidopsis GAMYB-like genes were obtained on the basis of sequence similarity to GAMYB genes from barley, rice, and L. temulentum. These three Arabadopsis genes were determined to encode transcription factors (AtMYB33, AtMYB65, and AtMYB101) and could substitute for a barley GAMYB and control alpha-amylase expression (Gocal et al. (2001) Plant Physiol. 127: 1682-1693).
(5) The floral control gene LEAFY from Arabidopsis can dramatically accelerate flowering in numerous dictoyledonous plants. Constitutive expression of Arabidopsis LEAFY also caused early flowering in transgenic rice (a monocot), with a heading date that was 26-34 days earlier than that of wild-type plants. These observations indicate that floral regulatory genes from Arabidopsis are useful tools for heading date improvement in cereal crops (He et al. (2000) Transgenic Res. 9: 223-227).
(6) Bioactive gibberellins (GAs) are essential endogenous regulators of plant growth. GA signaling tends to be conserved across the plant kingdom. GA signaling is mediated via GAI, a nuclear member of the GRAS family of plant transcription factors. Arabidopsis GAI has been shown to function in rice to inhibit gibberellin response pathways (Fu et al. (2001) Plant Cell 13: 1791-1802).
(7) The Arabidopsis gene SUPERMAN (SUP), encodes a putative transcription factor that maintains the boundary between stamens and carpels. By over-expressing Arabidopsis SUP in rice, the effect of the gene's presence on whorl boundaries was shown to be conserved. This demonstrated that SUP is a conserved regulator of floral whorl boundaries and affects cell proliferation (Nandi et al. (2000) Curr. Biol. 10: 215-218).
(8) Maize, petunia and Arabidopsis myb transcription factors that regulate flavonoid biosynthesis are very genetically similar and affect the same trait in their native species, therefore sequence and function of these myb transcription factors correlate with each other in these diverse species (Borevitz et al. (2000) Plant Cell 12: 2383-2394).
(9) Wheat reduced height-1 (Rht-B1/Rht-D1) and maize dwarf-8 (d8) genes are orthologs of the Arabidopsis gibberellin insensitive (GAI) gene. Both of these genes have been used to produce dwarf grain varieties that have improved grain yield. These genes encode proteins that resemble nuclear transcription factors and contain an SH2-like domain, indicating that phosphotyrosine may participate in gibberellin signaling. Transgenic rice plants containing a mutant GAI allele from Arabidopsis have been shown to produce reduced responses to gibberellin and are dwarfed, indicating that mutant GAI orthologs could be used to increase yield in a wide range of crop species (Peng et al. (1999) Nature 400: 256-261).

Transcription factors that are homologous to the listed sequences will typically share, in at least one conserved domain, at least about 75% amino acid sequence identity. At the nucleotide level, the sequences will typically share at least about 50% nucleotide sequence identity or more sequence identity to one or more of the listed sequences. The degeneracy of the genetic code enables major variations in the nucleotide sequence of a polynucleotide while maintaining the amino acid sequence of the encoded protein.

Percent identity can be determined electronically, e.g., by using the MEGALIGN program (DNASTAR, Inc. Madison, Wis.). The MEGALIGN program can create alignments between two or more sequences according to different methods, for example, the clustal method. (See, for example, Higgins and Sharp (1988) Gene 73: 237-244.) The clustal algorithm groups sequences into clusters by examining the distances between all pairs. The clusters are aligned pairwise and then in groups. Other alignment algorithms or programs may be used, including FASTA, BLAST, or ENTREZ, FASTA and BLAST, and that may be used to calculate percent similarity. These are available as a part of the GCG sequence analysis package (University of Wisconsin, Madison, Wis.), and can be used with or without default settings. ENTREZ is available through the National Center for Biotechnology Information. In one embodiment, the percent identity of two sequences can be determined by the GCG program with a gap weight of 1, e.g., each amino acid gap is weighted as if it were a single amino acid or nucleotide mismatch between the two sequences (see U.S. Pat. No. 6,262,333).

Other techniques for alignment are described in Doolittle, R. F. (1996) Methods in Enzymology: Computer Methods for Macromolecular Sequence Analysis, vol. 266, Academic Press, Orlando, Fla., USA. Preferably, an alignment program that permits gaps in the sequence is utilized to align the sequences. The Smith-Waterman is one type of algorithm that permits gaps in sequence alignments (see Shpaer (1997) Methods Mol. Biol. 70: 173-187). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

The percentage similarity between two polypeptide sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no similarity between the two amino acid sequences are not included in determining percentage similarity. Percent identity between polynucleotide sequences can also be counted or calculated by other methods known in the art, e.g., the Jotun Hein method. (See, for example, Hein (1990) Methods Enzymol. 183: 626-645.) Identity between sequences can also be determined by other methods known in the art, e.g., by varying hybridization conditions (see US Patent Application No. 20010010913).

Thus, the invention provides methods for identifying a sequence similar or paralogous or orthologous or homologous to one or more polynucleotides as noted herein, or one or more target polypeptides encoded by the polynucleotides, or otherwise noted herein and may include lining or associating a given plant phenotype or gene function with a sequence. In the methods, a sequence database is provided (locally or across an internet or intranet) and a query is made against the sequence database using the relevant sequences herein and associated plant phenotypes or gene functions.

In addition, one or more polynucleotide sequences or one or more polypeptides encoded by the polynucleotide sequences may be used to search against a BLOCKS (Bairoch et al. (1997) Nucleic Acids Res. 25: 217-221), PFAM, and other databases that contain previously identified and annotated motifs, sequences and gene functions. Methods that search for primary sequence patterns with secondary structure gap penalties (Smith et al. (1992) Protein Engineering 5: 35-51) as well as algorithms such as Basic Local Alignment Search Tool (BLAST; Altschul (1993) J. Mol. Evol. 36: 290-300; Altschul et al. (1990) J. Mol. Biol. 215: 403-410), BLOCKS (Henikoff and Henikoff (1991) Nucleic Acids Res. 19: 6565-6572), Hidden Markov Models (HMM; Eddy (1996) Curr. Opin. Str. Biol. 6: 361-365; Sonnhammer et al. (1997) Proteins 28: 405-420), and the like, can be used to manipulate and analyze polynucleotide and polypeptide sequences encoded by polynucleotides. These databases, algorithms and other methods are well known in the art and are described in Ausubel et al. (1997; Short Protocols in Molecular Biology, John Wiley & Sons, New York, N.Y., unit 7.7) and in Meyers (1995; Molecular Biology and Biotechnology, Wiley VCH, New York, N.Y., p 856-853).

Furthermore, methods using manual alignment of sequences similar or homologous to one or more polynucleotide sequences or one or more polypeptides encoded by the polynucleotide sequences may be used to identify regions of similarity and conserved domains. Such manual methods are well-known of those of skill in the art and can include, for example, comparisons of tertiary structure between a polypeptide sequence encoded by a polynucleotide that comprises a known function with a polypeptide sequence encoded by a polynucleotide sequence that has a function not yet determined. Such examples of tertiary structure may comprise predicted alpha helices, beta-sheets, amphipathic helices, leucine zipper motifs, zinc finger motifs, proline-rich regions, cysteine repeat motifs, and the like.

Orthologs and paralogs of presently disclosed transcription factors may be cloned using compositions provided by the present invention according to methods well known in the art. cDNAs may be cloned using mRNA from a plant cell or tissue that expresses one of the present transcription factors. Appropriate mRNA sources may be identified by interrogating Northern blots with probes designed from the present transcription factor sequences, after which a library is prepared from the mRNA obtained from a positive cell or tissue. Transcription factor-encoding cDNA is then isolated by, for example, PCR, using primers designed from a presently disclosed transcription factor gene sequence or by probing with a partial or complete cDNA or with one or more sets of degenerate probes based on the disclosed sequences. The cDNA library may be used to transform plant cells. Expression of the cDNAs of interest is detected using, for example, methods disclosed herein such as microarrays, Northern blots, quantitative PCR, or any other technique for monitoring changes in expression. Genomic clones may be isolated using similar techniques.

Examples of orthologs encoded by the Arabidopsis tdr polynucleotide sequences (SEQ ID NOs: 1 and 3) and TDR polypeptide sequences (SEQ ID NOs: 2 and 4) include, but are not limited to, SEQ ID NOs: 8, 10, 12, 14, 16, 18, 20, 22, 24, 26, 28, 30, 32, 34, 36, 38, 40, 42.

Identifying Polynucleotides or Nucleic Acids by Hybridization. Polynucleotides homologous to the sequences illustrated in the Sequence Listing and tables can be identified, e.g., by hybridization to each other under stringent or under highly stringent conditions. Single stranded polynucleotides hybridize when they associate based on a variety of well characterized physical-chemical forces, such as hydrogen bonding, solvent exclusion, base stacking and the like. The stringency of a hybridization reflects the degree of sequence identity of the nucleic acids involved, such that the higher the stringency, the more similar are the two polynucleotide strands. Stringency is influenced by a variety of factors, including temperature, salt concentration and composition, organic and non-organic additives, solvents, etc. present in both the hybridization and wash solutions and incubations (and number thereof), as described in more detail in the references cited above.

The invention encompasses polynucleotide sequences capable of hybridizing to the claimed polynucleotide sequences, including any of the transcription factor polynucleotides within the Sequence Listing, or fragments thereof under various conditions of stringency (Wahl and Berger (1987) Methods Enzymol. 152: 399-407; and Kimmel (1987) Methods Enzymol. 152: 507-511). In addition to the nucleotide sequences listed in the Sequence Listing and Tables, full length cDNA, orthologs, and paralogs of the present nucleotide sequences may be identified and isolated using well-known methods. The cDNA libraries, orthologs, and paralogs of the present nucleotide sequences may be screened using hybridization methods to determine their utility as hybridization target or amplification probes.

With regard to hybridization, conditions that are highly stringent, and means for achieving them, are well known in the art. See, for example, Sambrook et al. (1989) supra; Berger (1987) supra, pages 467-469; and Anderson and Young (1985) “Quantitative Filter Hybridisation.” In: Hames and Higgins, ed., Nucleic Acid Hybridisation, A Practical Approach. Oxford, IRL Press, 73-111.

Stability of DNA duplexes is affected by such factors as base composition, length, and degree of base pair mismatch. Hybridization conditions may be adjusted to allow DNAs of different sequence relatedness to hybridize. The melting temperature (Tₘ) is defined as the temperature when 50% of the duplex molecules have dissociated into their constituent single strands. The melting temperature of a perfectly matched duplex, where the hybridization buffer contains formamide as a denaturing agent, may be estimated by the following equations:

(I) DNA-DNA:
Tₘ(° C.)=81.5+16.6(log [Na+])+0.41(% G+C)−0.62(% formamide)−500/L
(I) DNA-RNA:
Tₘ(° C.)=79.8+18.5(log [Na+])+0.58(% G+C)+0.12(% G+C)²−0.5(% formamide)−820/L
(E) RNA-RNA:
Tₘ(° C.)=79.8+18.5(log [Na+])+0.58(% G+C)+0.12(% G+C)²−0.35(% formamide)−820/L

where L is the length of the duplex formed, [Na+] is the molar concentration of the sodium ion in the hybridization or washing solution, and % G+C is the percentage of (guanine+cytosine) bases in the hybrid. For imperfectly matched hybrids, approximately 1° C. is required to reduce the melting temperature for each 1% mismatch.

Hybridization experiments are generally conducted in a buffer of pH between 6.8 to 7.4, although the rate of hybridization is nearly independent of pH at ionic strengths likely to be used in the hybridization buffer (Anderson et al. (1985) supra). In addition, one or more of the following may be used to reduce non-specific hybridization: sonicated salmon sperm DNA or another non-complementary DNA, bovine serum albumin, sodium pyrophosphate, sodium dodecylsulfate (SDS), polyvinyl-pyrrolidone, ficoll and Denhardt's solution. Dextran sulfate and polyethylene glycol 6000 act to exclude DNA from solution, thus raising the effective probe DNA concentration and the hybridization signal within a given unit of time. In some instances, conditions of even greater stringency may be desirable or required to reduce non-specific and/or background hybridization. These conditions may be created with the use of higher temperature, lower ionic strength and higher concentration of a denaturing agent such as formamide.

Stringency conditions can be adjusted to screen for moderately similar fragments such as homologous sequences from distantly related organisms, or to highly similar fragments such as genes that duplicate functional enzymes from closely related organisms. The stringency can be adjusted either during the hybridization step or in the post-hybridization washes. Salt concentration, formamide concentration, hybridization temperature and probe lengths are variables that can be used to alter stringency (as described by the formula above). As a general guideline, high stringency is typically performed at Tₘ−5° C. to Tₘ−20° C., moderate stringency at Tₘ−20° C. to Tₘ−35° C. and low stringency at Tₘ−35° C. to Tₘ−50° C. for duplex >150 base pairs. Hybridization may be performed at low to moderate stringency (25-50° C. below Tₘ), followed by post-hybridization washes at increasing stringencies. Maximum rates of hybridization in solution are determined empirically to occur at Tₘ−25° C. for DNA-DNA duplex and Tₘ−15° C. for RNA-DNA duplex. Optionally, the degree of dissociation may be assessed after each wash step to determine the need for subsequent, higher stringency wash steps.

High stringency conditions may be used to select for nucleic acid sequences with high degrees of identity to the disclosed sequences. An example of stringent hybridization conditions obtained in a filter-based method such as a Southern or northern blot for hybridization of complementary nucleic acids that have more than 100 complementary residues is about 5° C. to 20° C. lower than the thermal melting point (Tₘ) for the specific sequence at a defined ionic strength and pH. Conditions used for hybridization may include about 0.02 M to about 0.15 M sodium chloride, about 0.5% to about 5% casein, about 0.02% SDS or about 0.1% N-aurylsarcosine, about 0.001 M to about 0.03 M sodium citrate, at hybridization temperatures between about 50° C. and about 70° C. More preferably, high stringency conditions are about 0.02 M sodium chloride, about 0.5% casein, about 0.02% SDS, about 0.001 M sodium citrate, at a temperature of about 50° C. Nucleic acid molecules that hybridize under stringent conditions will typically hybridize to a probe based on either the entire DNA molecule or selected portions, e.g., to a unique subsequence, of the DNA.

Stringent salt concentration will ordinarily be less than about 750 mM NaCl and 75 mM trisodium citrate. Increasingly stringent conditions may be obtained with less than about 500 mM NaCl and 50 mM trisodium citrate, to even greater stringency with less than about 250 mM NaCl and 25 mM trisodium citrate. Low stringency hybridization can be obtained in the absence of organic solvent, e.g., formamide, whereas high stringency hybridization may be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. with formamide present. Varying additional parameters, such as hybridization time, the concentration of detergent, e.g., sodium dodecyl sulfate (SDS) and ionic strength, are well known to those skilled in the art. Various levels of stringency are accomplished by combining these various conditions as needed.

The washing steps that follow hybridization may also vary in stringency; the post-hybridization wash steps primarily determine hybridization specificity, with the most critical factors being temperature and the ionic strength of the final wash solution. Wash stringency can be increased by decreasing salt concentration or by increasing temperature. Stringent salt concentration for the wash steps will preferably be less than about 30 mM NaCl and 3 mM trisodium citrate, and most preferably less than about 15 mM NaCl and 1.5 mM trisodium citrate.

Thus, hybridization and wash conditions that may be used to bind and remove polynucleotides with less than the desired homology to the nucleic acid sequences or their complements that encode the present transcription factors include, for example:

6×SSC at 65° C.;

50% formamide, 4×SSC at 42° C.; or

0.5×SSC, 0.1% SDS at 65° C.;

with, for example, two wash steps of 10-30 minutes each. Useful variations on these conditions will be readily apparent to those skilled in the art.

A person of skill in the art would not expect substantial variation among polynucleotide species encompassed within the scope of the present invention because the stringent conditions set forth in the above formulae yield structurally similar polynucleotides.

If desired, one may employ wash steps of even greater stringency, including about 0.2×SSC, 0.1% SDS at 65° C. and washing twice, each wash step being about 30 minutes, or about 0.1×SSC, 0.1% SDS at 65° C. and washing twice for 30 minutes. The temperature for the wash solutions will ordinarily be at least about 25° C., and for greater stringency at least about 42° C. Hybridization stringency may be increased further by using the same conditions as in the hybridization steps, with the wash temperature raised about 3° C. to about 5° C., and stringency may be increased even further by using the same conditions except the wash temperature is raised about 6° C. to about 9° C. For identification of less closely related homologs, wash steps may be performed at a lower temperature, e.g., 50° C.

An example of a low stringency wash step employs a solution and conditions of at least 25° C. in 30 mM NaCl, 3 mM trisodium citrate, and 0.1% SDS over 30 minutes. Greater stringency may be obtained at 42° C. in 15 mM NaCl, with 1.5 mM trisodium citrate, and 0.1% SDS over 30 minutes. Even higher stringency wash conditions are obtained at 65° C.-68° C. in a solution of 15 mM NaCl, 1.5 mM trisodium citrate, and 0.1% SDS. Wash procedures will generally employ at least two final wash steps. Additional variations on these conditions will be readily apparent to those skilled in the art (see, for example, US Patent Application No. 20010010913).

Stringency conditions can be selected such that an oligonucleotide that is fully complementary to the coding oligonucleotide hybridizes to the coding oligonucleotide with at least about a 5-10× higher signal to noise ratio than the ratio for hybridization of the perfectly complementary oligonucleotide to a nucleic acid encoding a transcription factor known as of the filing date of the application. It may be desirable to select conditions for a particular assay such that a higher signal to noise ratio, that is, about 15× or more, is obtained. Accordingly, a subject nucleic acid will hybridize to a unique coding oligonucleotide with at least a 2× or greater signal to noise ratio as compared to hybridization of the coding oligonucleotide to a nucleic acid encoding known polypeptide. The particular signal will depend on the label used in the relevant assay, e.g., a fluorescent label, a calorimetric label, a radioactive label, or the like. Labeled hybridization or PCR probes for detecting related polynucleotide sequences may be produced by oligolabeling, nick translation, end-labeling, or PCR amplification using a labeled nucleotide.

Encompassed by the invention are polynucleotide sequences that are capable of hybridizing to the claimed polynucleotide sequences, including, for example, SEQ ID NO: 9 (G3430), the complement of SEQ ID NO: 9, and fragments thereof under stringent conditions (see, e.g., Wahl and Berger (1987) Methods Enzymol. 152: 399-407; Kimmel (1987) Methods Enzymol. 152: 507-511). Estimates of homology are provided by either DNA-DNA or DNA-RNA hybridization under conditions of stringency as is well understood by those skilled in the art (Hames and Higgins, Eds. (1985) Nucleic Acid Hybridisation, IRL Press, Oxford, U.K.). Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions.

Identifying Polynucleotides or Nucleic Acids with Expression Libraries. In addition to hybridization methods, transcription factor homolog polypeptides can be obtained by screening an expression library using antibodies specific for one or more transcription factors. With the provision herein of the disclosed transcription factor, and transcription factor homolog nucleic acid sequences, the encoded polypeptide(s) can be expressed and purified in a heterologous expression system (for example, E. coli) and used to raise antibodies (monoclonal or polyclonal) specific for the polypeptide(s) in question. Antibodies can also be raised against synthetic peptides derived from transcription factor, or transcription factor homolog, amino acid sequences. Methods of raising antibodies are well known in the art and are described in Harlow and Lane (1988), Antibodies: A Laboratory Manual, Cold Spring Harbor Laboratory, New York. Such antibodies can then be used to screen an expression library produced from the plant from which it is desired to clone additional transcription factor homologs, using the methods described above. The selected cDNAs can be confirmed by sequencing and enzymatic activity.

Sequence Variations. It will readily be appreciated by those of skill in the art, that any of a variety of polynucleotide sequences are capable of encoding the transcription factors and transcription factor homolog polypeptides of the invention. Due to the degeneracy of the genetic code, many different polynucleotides can encode identical and/or substantially similar polypeptides in addition to those sequences illustrated in the Sequence Listing. Nucleic acids having a sequence that differs from the sequences shown in the Sequence Listing, or complementary sequences, that encode functionally equivalent peptides (i.e., peptides having some degree of equivalent or similar biological activity) but differ in sequence from the sequence shown in the Sequence Listing due to degeneracy in the genetic code, are also within the scope of the invention.

Altered polynucleotide sequences encoding polypeptides include those sequences with deletions, insertions, or substitutions of different nucleotides, resulting in a polynucleotide encoding a polypeptide with at least one functional characteristic of the instant polypeptides. Included within this definition are polymorphisms that may or may not be readily detectable using a particular oligonucleotide probe of the polynucleotide encoding the instant polypeptides, and improper or unexpected hybridization to allelic variants, with a locus other than the normal chromosomal locus for the polynucleotide sequence encoding the instant polypeptides.

Allelic variant refers to any of two or more alternative forms of a gene occupying the same chromosomal locus. Allelic variation arises naturally through mutation, and may result in phenotypic polymorphism within populations. Gene mutations can be silent (i.e., no change in the encoded polypeptide) or may encode polypeptides having altered amino acid sequence. The term allelic variant is also used herein to denote a protein encoded by an allelic variant of a gene. Splice variant refers to alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a protein encoded by a splice variant of an mRNA transcribed from a gene.

Those skilled in the art would recognize that, for example, G3430, SEQ ID NO: 10, represents a single transcription factor; allelic variation and alternative splicing may be expected to occur. Allelic variants of SEQ ID NO: 9 can be cloned by probing cDNA or genomic libraries from different individual organisms according to standard procedures. Allelic variants of the DNA sequence shown in SEQ ID NO: 9, including those containing silent mutations and those in which mutations result in amino acid sequence changes, are within the scope of the present invention, as are proteins that are allelic variants of SEQ ID NO: 10. cDNAs generated from alternatively spliced mRNAs, which retain the properties of the transcription factor are included within the scope of the present invention, as are polypeptides encoded by such cDNAs and mRNAs. Allelic variants and splice variants of these sequences can be cloned by probing cDNA or genomic libraries from different individual organisms or tissues according to standard procedures known in the art (see U.S. Pat. No. 6,388,064).

Thus, in addition to the sequences set forth in the Sequence Listing (except CBF sequences), the invention also encompasses related nucleic acid molecules that include allelic or splice variants of SEQ ID NOs: 1, 3, 7, 9, 11, 13, 15, 17, 19, 21, 23, 25, 27, 29, 31, 33, 35, 37, 39, 41, 59, and include sequences that are complementary to any of the above nucleotide sequences. Related nucleic acid molecules also include nucleotide sequences encoding a polypeptide comprising a substitution, modification, addition and/or deletion of one or more amino acid residues compared to the polypeptide as set forth in any of SEQ ID NOs: 2, 4, 8, 10, 12, 14, 16, 18, 20, 22, 24, 26, 28, 30, 32, 34, 36, 38, 40, 42 and 60. Such related polypeptides may comprise, for example, additions and/or deletions of one or more N-linked or O-linked glycosylation sites, or an addition and/or a deletion of one or more cysteine residues.

For example, Table 3 illustrates, for example, that the codons AGC, AGT, TCA, TCC, TCG, and TCT all encode the same amino acid: serine. Accordingly, at each position in the sequence where there is a codon encoding serine, any of the above trinucleotide sequences can be used without altering the encoded polypeptide.

TABLE 3
Codons encoding amino acids

| Amino acid | | | Possible Codons | | | | | |
|---|---|---|---|---|---|---|---|---|
| Alanine | Ala | A | GCA | GCC | GCG | GCU | | |
| Cysteine | Cys | C | TGC | TGT | | | | |
| Aspartic acid | Asp | D | GAC | GAT | | | | |
| Glutamic acid | Glu | E | GAA | GAG | | | | |
| Phenylalanine | Phe | F | TTC | TTT | | | | |
| Glycine | Gly | G | GGA | GGC | GGG | GGT | | |
| Histidine | His | H | CAC | CAT | | | | |
| Isoleucine | Ile | I | ATA | ATC | ATT | | | |
| Lysine | Lys | K | AAA | AAG | | | | |
| Leucine | Leu | L | TTA | TTG | CTA | CTC | CTG | CTT |
| Methionine | Met | M | ATG | | | | | |
| Asparagine | Asn | N | AAC | AAT | | | | |
| Proline | Pro | P | CCA | CCC | CCG | CCT | | |
| Glutamine | Gln | Q | CAA | CAG | | | | |
| Arginine | Arg | R | AGA | AGG | CGA | CGC | CGG | CGT |
| Serine | Ser | S | AGC | AGT | TCA | TCC | TCG | TCT |
| Threonine | Thr | T | ACA | ACC | ACG | ACT | | |
| Valine | Val | V | GTA | GTC | GTG | GTT | | |
| Tryptophan | Trp | W | TGG | | | | | |
| Tyrosine | Tyr | Y | TAC | TAT | | | | |

Sequence alterations that do not change the amino acid sequence encoded by the polynucleotide are termed “silent” variations. With the exception of the codons ATG and TGG, encoding methionine and tryptophan, respectively, any of the possible codons for the same amino acid can be substituted by a variety of techniques, e.g., site-directed mutagenesis, available in the art. Accordingly, any and all such variations of a sequence selected from the above table are a feature of the invention.

In addition to silent variations, other conservative variations that alter one, or a few amino acids in the encoded polypeptide, can be made without altering the function of the polypeptide, these conservative variants are, likewise, a feature of the invention.

For example, substitutions, deletions and insertions introduced into the sequences provided in the Sequence Listing, are also envisioned by the invention. Such sequence modifications can be engineered into a sequence by site-directed mutagenesis (Wu, editor; Methods Enzymol. (1993) vol. 217, Academic Press) or the other methods noted below. Amino acid substitutions are typically of single residues; insertions usually will be on the order of about from 1 to 10 amino acid residues; and deletions will range about from 1 to 30 residues. In preferred embodiments, deletions or insertions are made in adjacent pairs, e.g., a deletion of two residues or insertion of two residues. Substitutions, deletions, insertions or any combination thereof can be combined to arrive at a sequence. The mutations that are made in the polynucleotide encoding the transcription factor should not place the sequence out of reading frame and should not create complementary regions that could produce secondary mRNA structure. Preferably, the polypeptide encoded by the DNA performs the desired function.

Conservative substitutions are those in which at least one residue in the amino acid sequence has been removed and a different residue inserted in its place. Such substitutions generally are made in accordance with the Table 4 when it is desired to maintain the activity of the protein. In one embodiment, a transcription factors listed in the Sequence Listing may have up to ten conservative substitutions and retain their function. In another embodiment, transcription factors listed in the Sequence Listing may have more than ten conservative substitutions and still retain their function.

TABLE 4
Conservative substitutions of amino acids

| | Conservative |
|---|---|
| Residue | Substitutions |
| Ala | Ser |
| Arg | Lys |
| Asn | Gln; His |
| Asp | Glu |
| Gln | Asn |
| Cys | Ser |
| Glu | Asp |
| Gly | Pro |
| His | Asn; Gln |
| Ile | Leu, Val |
| Leu | Ile; Val |
| Lys | Arg; Gln |
| Met | Leu; Ile |
| Phe | Met; Leu; Tyr |
| Ser | Thr; Gly |
| Thr | Ser; Val |
| Trp | Tyr |
| Tyr | Trp; Phe |
| Val | Ile; Leu |

Similar substitutions are those in which at least one residue in the amino acid sequence has been removed and a different residue inserted in its place. Such substitutions may be made in accordance with the Table 5 when it is desired to maintain the activity of the protein. Table 5 shows amino acids that can be substituted for an amino acid in a protein and that are typically regarded as structural and functional substitutions. For example, a residue in column 1 of Table 5 may be substituted with a residue in column 2; in addition, a residue in column 2 of Table 5 may be substituted with the residue of column 1.

TABLE 5
Similar substitutions of amino acids

| Residue | Similar Substitutions |
|---|---|
| Ala | Ser; Thr; Gly; Val; Leu; Ile |
| Arg | Lys; His; Gly |
| Asn | Gln; His; Gly; Ser; Thr |
| Asp | Glu, Ser; Thr |
| Gln | Asn; Ala |
| Cys | Ser; Gly |
| Glu | Asp |
| Gly | Pro; Arg |
| His | Asn; Gln; Tyr; Phe; Lys; Arg |
| Ile | Ala; Leu; Val; Gly; Met |
| Leu | Ala; Ile; Val; Gly; Met |
| Lys | Arg; His; Gln; Gly; Pro |
| Met | Leu; Ile; Phe |
| Phe | Met; Leu; Tyr; Trp; His; Val; Ala |
| Ser | Thr; Gly; Asp; Ala; Val; Ile; His |
| Thr | Ser; Val; Ala; Gly |
| Trp | Tyr; Phe; His |
| Tyr | Trp; Phe; His |
| Val | Ala; Ile; Leu; Gly; Thr; Ser; Glu |

Substitutions that are less conservative than those in Table 5 can be selected by picking residues that differ more significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. The substitutions that in general are expected to produce the greatest changes in protein properties will be those in which (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine.

Further Modifying Sequences of the Invention—Mutation/Forced Evolution. In addition to generating silent or conservative substitutions as noted, above, the present invention optionally includes methods of modifying the sequences of the Sequence Listing. In the methods, nucleic acid or protein modification methods are used to alter the given sequences to produce new sequences and/or to chemically or enzymatically modify given sequences to change the properties of the nucleic acids or proteins.

Thus, in one embodiment, given nucleic acid sequences are modified, e.g., according to standard mutagenesis or artificial evolution methods to produce modified sequences. The modified sequences may be created using purified natural polynucleotides isolated from any organism or may be synthesized from purified compositions and chemicals using chemical means well know to those of skill in the art. For example, Ausubel (2000) supra, provides additional details on mutagenesis methods. Artificial forced evolution methods are described, for example, by Stemmer (1994; Nature 370: 389-391), Stemmer (1994; Proc. Natl. Acad. Sci. USA 91: 10747-10751), and U.S. Pat. Nos. 5,811,238, 5,837,500, and 6,242,568. Methods for engineering synthetic transcription factors and other polypeptides are described, for example, by Zhang et al. (2000) J. Biol. Chem. 275: 33850-33860, Liu et al. (2001) J. Biol. Chem. 276: 11323-11334, and Isalan et al. (2001) Nature Biotechnol. 19: 656-660. Many other mutation and evolution methods are also available and expected to be within the skill of the practitioner.

Similarly, chemical or enzymatic alteration of expressed nucleic acids and polypeptides can be performed by standard methods. For example, sequence can be modified by addition of lipids, sugars, peptides, organic or inorganic compounds, by the inclusion of modified nucleotides or amino acids, or the like. For example, protein modification techniques are illustrated in Ausubel (2000) supra. Further details on chemical and enzymatic modifications can be found herein. These modification methods can be used to modify any given sequence, or to modify any sequence produced by the various mutation and artificial evolution modification methods noted herein.

Accordingly, the invention provides for modification of any given nucleic acid by mutation, evolution, chemical or enzymatic modification, or other available methods, as well as for the products produced by practicing such methods, e.g., using the sequences herein as a starting substrate for the various modification approaches.

For example, optimized coding sequence containing codons preferred by a particular prokaryotic or eukaryotic host can be used e.g., to increase the rate of translation or to produce recombinant RNA transcripts having desirable properties, such as a longer half-life, as compared with transcripts produced using a non-optimized sequence. Translation stop codons can also be modified to reflect host preference. For example, preferred stop codons for Saccharomyces cerevisiae and mammals are TAA and TGA, respectively. The preferred stop codon for monocotyledonous plants is TGA, whereas insects and E. coli prefer to use TAA as the stop codon.

The polynucleotide sequences of the present invention can also be engineered in order to alter a coding sequence for a variety of reasons, including but not limited to, alterations that modify the sequence to facilitate cloning, processing and/or expression of the gene product. For example, alterations are optionally introduced using techniques that are well known in the art, e.g., site-directed mutagenesis, to insert new restriction sites, to alter glycosylation patterns, to change codon preference, to introduce splice sites, etc.

Furthermore, a fragment or domain derived from any of the polypeptides of the invention can be combined with domains derived from other transcription factors or synthetic domains to modify the biological activity of a transcription factor. For instance, a DNA-binding domain derived from a transcription factor of the invention can be combined with the activation domain of another transcription factor or with a synthetic activation domain. A transcription activation domain assists in initiating transcription from a DNA-binding site. Examples include the transcription activation region of VP16 or GAL4 (Moore et al. (1998) Proc. Natl. Acad. Sci. USA 95: 376-381; Aoyama et al. (1995) Plant Cell 7: 1773-1785), peptides derived from bacterial sequences (Ma and Ptashne (1987) Cell 51: 113-119) and synthetic peptides (Giniger and Ptashne (1987) Nature 330: 670-672).

Expression and Modification of Polypeptides. Typically, polynucleotide sequences of the invention are incorporated into recombinant DNA (or RNA) molecules that direct expression of polypeptides of the invention in appropriate host cells, transgenic plants, in vitro translation systems, or the like. Due to the inherent degeneracy of the genetic code, nucleic acid sequences that encode substantially the same or a functionally equivalent amino acid sequence can be substituted for any listed sequence to provide for cloning and expressing the relevant homolog.

The transgenic plants of the present invention comprising recombinant polynucleotide sequences are generally derived from parental plants, which may themselves be non-transformed (or non-transgenic) plants. These transgenic plants may either have a transcription factor gene “knocked out” (for example, with a genomic insertion by homologous recombination, an antisense or ribozyme construct) or expressed to a normal or wild-type extent. However, overexpressing transgenic “progeny” plants will exhibit greater mRNA levels, wherein the mRNA encodes a transcription factor, that is, a DNA-binding protein that is capable of binding to a DNA regulatory sequence and inducing transcription, and preferably, expression of a plant trait gene. Preferably, the mRNA expression level will be at least three-fold greater than that of the parental plant, or more preferably at least ten-fold greater mRNA levels compared to said parental plant, and most preferably at least fifty-fold greater compared to said parental plant.

Vectors Promoters, and Expression Systems. The present invention includes recombinant constructs comprising one or more of the nucleic acid sequences herein. The constructs typically comprise a vector, such as a plasmid, a cosmid, a phage, a virus (e.g., a plant virus), a bacterial artificial chromosome (BAC), a yeast artificial chromosome (YAC), or the like, into which a nucleic acid sequence of the invention has been inserted, in a forward or reverse orientation. In a preferred aspect of this embodiment, the construct further comprises regulatory sequences, including, for example, a promoter, operably linked to the sequence. Large numbers of suitable vectors and promoters are known to those of skill in the art, and are commercially available.

General texts that describe molecular biological techniques useful herein, including the use and production of vectors, promoters and many other relevant topics, include Berger (1987) supra, Sambrook et al. (1989) supra, and Ausubel (2000) supra. Any of the identified sequences can be incorporated into a cassette or vector, e.g., for expression in plants. A number of expression vectors suitable for stable transformation of plant cells or for the establishment of transgenic plants have been described including those described in Weissbach and Weissbach (1989) Methods for Plant Molecular Biology, Academic Press, and Gelvin et al. (1990) Plant Molecular Biology Manual, Kluwer Academic Publishers. Specific examples include those derived from a Ti plasmid of Agrobacterium tumefaciens, as well as those disclosed by Herrera-Estrella et al. (1983) Nature 303: 209, Bevan (1984) Nucleic Acids Res. 12: 8711-8721, Klee (1985) Bio/Technology 3: 637-642, for dicotyledonous plants.

Alternatively, non-Ti vectors can be used to transfer the DNA into monocotyledonous plants and cells by using free DNA delivery techniques. Such methods can involve, for example, the use of liposomes, electroporation, microprojectile bombardment, silicon carbide whiskers, and viruses. By using these methods transgenic plants such as wheat, rice (Christou (1991) Bio/Technology 9: 957-962) and corn (Gordon-Kamm (1990) Plant Cell 2: 603-618) can be produced. An immature embryo can also be a good target tissue for monocots for direct DNA delivery techniques by using the particle gun (Weeks et al. (1993) Plant Physiol. 102: 1077-1084; Vasil (1993) Bio/Technology 10: 667-674; Wan and Lemeaux (1994) Plant Physiol. 104: 37-48, and for Agrobacterium-mediated DNA transfer (Ishida et al. (1996) Nature Biotechnol. 14: 745-750).

Typically, plant transformation vectors include one or more cloned plant coding sequence (genomic or cDNA) under the transcriptional control of 5′ and 3′ regulatory sequences and a dominant selectable marker. Such plant transformation vectors typically also contain a promoter (e.g., a regulatory region controlling inducible or constitutive, environmentally- or developmentally-regulated, or cell- or tissue-specific expression), a transcription initiation start site, an RNA processing signal (such as intron splice sites), a transcription termination site, and/or a polyadenylation signal.

A potential utility for the transcription factor polynucleotides disclosed herein is the isolation of promoter elements from these genes that can be used to program expression in plants of any genes. Each transcription factor gene disclosed herein is expressed in a unique fashion, as determined by promoter elements located upstream of the start of translation, and additionally within an intron of the transcription factor gene or downstream of the termination codon of the gene. As is well known in the art, for a significant portion of genes, the promoter sequences are located entirely in the region directly upstream of the start of translation. In such cases, typically the promoter sequences are located within 2.0 kb of the start of translation, or within 1.5 kb of the start of translation, frequently within 1.0 kb of the start of translation, and sometimes within 0.5 kb of the start of translation.

The promoter sequences can be isolated according to methods known to one skilled in the art.

Examples of constitutive plant promoters that can be useful for expressing the transcription factor sequence include: the cauliflower mosaic virus (CaMV) 35S promoter, which confers constitutive, high-level expression in most plant tissues (see, for example, Odell et al. (1985) Nature 313: 810-812); the nopaline synthase promoter (An et al. (1988) Plant Physiol. 88: 547-552); and the octopine synthase promoter (Fromm et al. (1989) Plant Cell 1: 977-984).

A variety of plant gene promoters that regulate gene expression in response to environmental, hormonal, chemical, developmental signals, and in a tissue-active manner can be used for expression of a transcription factor sequence in plants. Choice of a promoter is based largely on the phenotype of interest and is determined by such factors as tissue (e.g., seed, fruit, root, pollen, vascular tissue, flower, carpel, etc.), inducibility (e.g., in response to wounding, heat, cold, drought, light, pathogens, etc.), timing, developmental stage, and the like. Numerous known promoters have been characterized and can favorably be employed to promote expression of a polynucleotide of the invention in a transgenic plant or cell of interest. For example, tissue specific promoters include: seed-specific promoters (such as the napin, phaseolin or DC3 promoter described in U.S. Pat. No. 5,773,697), fruit-specific promoters that are active during fruit ripening (such as the dru 1 promoter (U.S. Pat. No. 5,783,393), or the 2A11 promoter (U.S. Pat. No. 4,943,674) and the tomato polygalacturonase promoter (Bird et al. (1988) Plant Mol. Biol. 11: 651-662), root-specific promoters, such as those disclosed in U.S. Pat. Nos. 5,618,988, 5,837,848 and 5,905,186, pollen-active promoters such as PTA29, PTA26 and PTA13 (U.S. Pat. No. 5,792,929), promoters active in vascular tissue (Ringli and Keller (1998) Plant Mol. Biol. 37: 977-988), flower-specific (Kaiser et al. (1995) Plant Mol. Biol. 28: 231-243), pollen (Baerson et al. (1994) Plant Mol. Biol. 26: 1947-1959), carpels (Ohl et al. (1990) Plant Cell 2: 837-848), pollen and ovules (Baerson et al. (1993) Plant Mol. Biol. 22: 255-267), auxin-inducible promoters (such as that described in van der Kop et al. (1999) Plant Mol. Biol. 39: 979-990 or Baumann et al. (1999) Plant Cell 11: 323-334), cytokinin-inducible promoter (Guevara-Garcia (1998) Plant Mol. Biol. 38: 743-753), promoters responsive to gibberellin (Shi et al. (1998) Plant Mol. Biol. 38: 1053-1060, Willmott et al. (1998) Plant Mol. Biol. 38: 817-825) and the like. Additional promoters are those that elicit expression in response to heat (Ainley et al. (1993) Plant Mol. Biol. 22: 13-23), light (e.g., the pea rbcS-3A promoter, Kuhlemeier et al. (1989) Plant Cell 1: 471-478, and the maize rbcS promoter, Schaffner and Sheen (1991) Plant Cell 3: 997-1012); wounding (e.g., wunI, Siebertz et al. (1989) Plant Cell 1: 961-968); pathogens (such as the PR-1 promoter described in Buchel et al. (1999) Plant Mol. Biol. 40: 387-396, and the PDF1.2 promoter described in Manners et al. (1998) Plant Mol. Biol. 38: 1071-1080), and chemicals such as methyl jasmonate or salicylic acid (Gatz (1997) Annu. Rev. Plant Physiol. Plant Mol. Biol. 48: 89-108). In addition, the timing of the expression can be controlled by using promoters such as those acting at senescence (Gan and Amasino (1995) Science 270: 1986-1988); or late seed development (Odell et al. (1994) Plant Physiol. 106: 447-458).

Plant expression vectors can also include RNA processing signals that can be positioned within, upstream or downstream of the coding sequence. In addition, the expression vectors can include additional regulatory sequences from the 3′-untranslated region of plant genes, e.g., a 3′ terminator region to increase mRNA stability of the mRNA, such as the PI-II terminator region of potato or the octopine or nopaline synthase 3′ terminator regions.

Additional Expression Elements. Specific initiation signals can aid in efficient translation of coding sequences. These signals can include, e.g., the ATG initiation codon and adjacent sequences. In cases where a coding sequence, its initiation codon and upstream sequences are inserted into the appropriate expression vector, no additional translational control signals may be needed. However, in cases where only coding sequence (e.g., a mature protein coding sequence), or a portion thereof, is inserted, exogenous transcriptional control signals including the ATG initiation codon can be separately provided. The initiation codon is provided in the correct reading frame to facilitate transcription. Exogenous transcriptional elements and initiation codons can be of various origins, both natural and synthetic. The efficiency of expression can be enhanced by the inclusion of enhancers appropriate to the cell system in use.

Expression Hosts. The present invention also relates to host cells that are transduced with vectors of the invention, and the production of polypeptides of the invention (including fragments thereof) by recombinant techniques. Host cells are genetically engineered (i.e., nucleic acids are introduced, e.g., transduced, transformed or transfected) with the vectors of this invention, which may be, for example, a cloning vector or an expression vector comprising the relevant nucleic acids herein. The vector is optionally a plasmid, a viral particle, a phage, a naked nucleic acid, etc. The engineered host cells can be cultured in conventional nutrient media modified as appropriate for activating promoters, selecting transformants, or amplifying the relevant gene. The culture conditions, such as temperature, pH and the like, are those previously used with the host cell selected for expression, and will be apparent to those skilled in the art and in the references cited herein, including, Sambrook et al. (1989) supra and Ausubel (2000) supra.

The host cell can be a eukaryotic cell, such as a yeast cell, or a plant cell, or the host cell can be a prokaryotic cell, such as a bacterial cell. Plant protoplasts are also suitable for some applications. For example, the DNA fragments are introduced into plant tissues, cultured plant cells or plant protoplasts by standard methods including electroporation (Fromm et al. (1985) Proc. Natl. Acad. Sci. USA 82: 5824-5828, infection by viral vectors such as cauliflower mosaic virus (Hohn et al. (1982) Molecular Biology of Plant Tumors, Academic Press, New York, N.Y., pp. 549-560; U.S. Pat. No. 4,407,956), high velocity ballistic penetration by small particles with the nucleic acid either within the matrix of small beads or particles, or on the surface (Klein et al. (1987) Nature 327: 70-73), use of pollen as vector (WO 85/01856), or use of Agrobacterium tumefaciens or A. rhizogenes carrying a T-DNA plasmid in which DNA fragments are cloned. The T-DNA plasmid is transmitted to plant cells upon infection by Agrobacterium tumefaciens, and a portion is stably integrated into the plant genome (Horsch et al. (1984) Science 233: 496-498; Fraley et al. (1983) Proc. Natl. Acad. Sci. USA 80: 4803-4807).

The cell can include a nucleic acid of the invention that encodes a polypeptide, wherein the cell expresses a polypeptide of the invention. The cell can also include vector sequences, or the like. Furthermore, cells and transgenic plants that include any polypeptide or nucleic acid above or throughout this specification, e.g., produced by transduction of a vector of the invention, are an additional feature of the invention.

For long-term, high-yield production of recombinant proteins, stable expression can be used. Host cells transformed with a nucleotide sequence encoding a polypeptide of the invention are optionally cultured under conditions suitable for the expression and recovery of the encoded protein from cell culture. The protein or fragment thereof produced by a recombinant cell may be secreted, membrane-bound, or contained intracellularly, depending on the sequence and/or the vector used. As will be understood by those of skill in the art, expression vectors containing polynucleotides encoding mature proteins of the invention can be designed with signal sequences that direct secretion of the mature polypeptides through a prokaryotic or eukaryotic cell membrane.

Modified Amino Acid Residues. Polypeptides of the invention may contain one or more modified amino acid residues. The presence of modified amino acids may be advantageous in, for example, increasing polypeptide half-life, reducing polypeptide antigenicity or toxicity, increasing polypeptide storage stability, or the like. Amino acid residue(s) are modified, for example, co-translationally or post-translationally during recombinant production or modified by synthetic or chemical means.

Non-limiting examples of a modified amino acid residue include incorporation or other use of acetylated amino acids, glycosylated amino acids, sulfated amino acids, prenylated (e.g., farnesylated, geranylgeranylated) amino acids, PEG modified (for example, “PEGylated”) amino acids, biotinylated amino acids, carboxylated amino acids, phosphorylated amino acids, etc. References adequate to guide one of skill in the modification of amino acid residues are replete throughout the literature.

The modified amino acid residues may prevent or increase affinity of the polypeptide for another molecule, including, but not limited to, polynucleotide, proteins, carbohydrates, lipids and lipid derivatives, and other organic or synthetic compounds.

Identification of Additional Factors. A transcription factor provided by the present invention can also be used to identify additional endogenous or exogenous molecules that can affect a phentoype or trait of interest. On the one hand, such molecules include organic (small or large molecules) and/or inorganic compounds that modulate expression of (i.e., regulate) a particular transcription factor. Alternatively, such molecules include endogenous molecules that are acted upon either at a transcriptional level by a transcription factor of the invention to modify a phenotype as desired. For example, the transcription factors can be employed to identify one or more downstream genes that are subject to a regulatory effect of the transcription factor. In one approach, a transcription factor or transcription factor homolog of the invention is expressed in a host cell, e.g., a transgenic plant cell, tissue or explant, and expression products, either RNA or protein, of likely or random targets are monitored, e.g., by hybridization to a microarray of nucleic acid probes corresponding to genes expressed in a tissue or cell type of interest, by two-dimensional gel electrophoresis of protein products, or by any other method known in the art for assessing expression of gene products at the level of RNA or protein. Alternatively, a transcription factor of the invention can be used to identify promoter sequences (such as binding sites on DNA sequences) involved in the regulation of a downstream target. After identifying a promoter sequence, interactions between the transcription factor and the promoter sequence can be modified by changing specific nucleotides in the promoter sequence or specific amino acids in the transcription factor that interact with the promoter sequence to alter a plant trait. Typically, transcription factor DNA-binding sites are identified by gel shift assays. After identifying the promoter regions, the promoter region sequences can be employed in double-stranded DNA arrays to identify molecules that affect the interactions of the transcription factors with their promoters (Bulyk et al. (1999) Nature Biotechnol. 17: 573-577).

The identified transcription factors are also useful to identify proteins that modify the activity of the transcription factor. Such modification can occur by covalent modification, such as by phosphorylation, or by protein-protein (homo or -heteropolymer) interactions. Any method suitable for detecting protein-protein interactions can be employed. Among the methods that can be employed are co-immunoprecipitation, cross-linking and co-purification through gradients or chromatographic columns, and the two-hybrid yeast system.

The two-hybrid system detects protein interactions in vivo and is described in Chien et al. (1991) Proc. Natl. Acad. Sci. USA 88: 9578-9582, and is commercially available from Clontech (Palo Alto, Calif.). In such a system, plasmids are constructed that encode two hybrid proteins: one consists of the DNA-binding domain of a transcription activator protein fused to the transcription factor polypeptide and the other consists of the transcription activator protein's activation domain fused to an unknown protein that is encoded by a cDNA that has been recombined into the plasmid as part of a cDNA library. The DNA-binding domain fusion plasmid and the cDNA library are transformed into a strain of the yeast Saccharomyces cerevisiae that contains a reporter gene (e.g., lacZ) whose regulatory region contains the transcription activator's binding site. Either hybrid protein alone cannot activate transcription of the reporter gene. Interaction of the two hybrid proteins reconstitutes the functional activator protein and results in expression of the reporter gene, which is detected by an assay for the reporter gene product. Then, the library plasmids responsible for reporter gene expression are isolated and sequenced to identify the proteins encoded by the library plasmids. After identifying proteins that interact with the transcription factors, assays for compounds that interfere with the transcription factor protein-protein interactions can be performed.

Subsequences. Also contemplated are uses of polynucleotides, also referred to herein as oligonucleotides, typically having at least 12 bases, preferably at least 15, more preferably at least 20, 30, or 50 bases, which hybridize under stringent conditions to a polynucleotide sequence described above. The polynucleotides may be used as probes, primers, sense and antisense agents, and the like, according to methods as noted supra.

Subsequences of the polynucleotides of the invention, including polynucleotide fragments and oligonucleotides are useful as nucleic acid probes and primers. An oligonucleotide suitable for use as a probe or primer is at least about 15 nucleotides in length, more often at least about 18 nucleotides, often at least about 21 nucleotides, frequently at least about 30 nucleotides, or about 40 nucleotides, or more in length. A nucleic acid probe is useful in hybridization protocols, for example, to identify additional polypeptide homologs of the invention, including protocols for microarray experiments. Primers can be annealed to a complementary target DNA strand by nucleic acid hybridization to form a hybrid between the primer and the target DNA strand, and then extended along the target DNA strand by a DNA polymerase enzyme. Primer pairs can be used for amplification of a nucleic acid sequence, e.g., by the polymerase chain reaction (PCR) or other nucleic-acid amplification methods. See Sambrook et al. (1989) supra, and Ausubel (2000) supra.

In addition, the invention includes an isolated or recombinant polypeptide including a subsequence of at least about 15 contiguous amino acids encoded by the recombinant or isolated polynucleotides of the invention. For example, such polypeptides, or domains or fragments thereof, can be used as immunogens, e.g., to produce antibodies specific for the polypeptide sequence, or as probes for detecting a sequence of interest. A subsequence can range in size from about 15 amino acids in length up to and including the fall length of the polypeptide.

To be encompassed by the present invention, an expressed polypeptide that comprises such a polypeptide subsequence performs at least one biological function of the intact polypeptide in substantially the same manner, or to a similar extent, as does the intact polypeptide. For example, a polypeptide fragment can comprise a recognizable structural motif or functional domain such as a DNA binding domain that activates transcription, for example, by binding to a specific DNA promoter region an activation domain, or a domain for protein-protein interactions.

Traits That May Be Modified in Overexpressing or Knock-out Plants. Presently disclosed transcription factor genes, including G28, G3430 and their equivalogs, have been shown to or are likely to affect a plant's response to various plant diseases, pathogens and pests, and may increase the tolerance or resistance of a plant to more than one pathogen. The pathogenic organisms include, for example, fungal pathogens Fusarium oxysporum, Botrytis cinerea, Sclerotinia sclerotiorum, and Erysiphe orontii. Bacterial pathogens to which resistance may be conferred include Pseudomonas syringae. Other problem organisms may potentially include nematodes, mollicutes, parasites, or herbivorous arthropods. In each case, overexpression of one or more of the transcription factor sequences of the invention may provide benefit to the plant to help prevent or overcome infestation, or be used to manipulate any of the various plant responses to disease. These mechanisms by which the transcription factors work could include increasing surface waxes or oils, surface thickness, or the activation of signal transduction pathways that regulate plant defense in response to attacks by herbivorous pests (including, for example, protease inhibitors). Another means to combat fungal and other pathogens is by accelerating local cell death or senescence, mechanisms used to impair the spread of pathogenic microorganisms throughout a plant. For instance, the best known example of accelerated cell death is the resistance gene-mediated hypersensitive response, which causes localized cell death at an infection site and initiates a systemic defense response. Because many defenses, signaling molecules, and signal transduction pathways are common to defense against different pathogens and pests, such as fungal, bacterial, oomycete, nematode, and insect, transcription factors that are implicated in defense responses against the fungal pathogens tested may also function in defense against other pathogens and pests. For example, the transcription factor from tobacco, Tsi1 (Shin et al. (2002) Mol. Plant-Microbe Interactions 15: 939-989) provides improved resistance in pepper plants to a fungal pathogen (Phtyophthora capsici), a bacterial pathogen (Xanthomonas campestris) and a viral pathogen (cucumber mosaic virus).

Production of Transgenic Plants

Modification of Traits. The polynucleotides of the invention are favorably employed to produce transgenic plants with various traits, or characteristics, that have been modified in a desirable manner, e.g., to improve the seed characteristics of a plant. For example, alteration of expression levels or patterns (e.g., spatial or temporal expression patterns) of one or more of the transcription factors (or transcription factor homologs) of the invention, as compared with the levels of the same protein found in a wild-type plant, can be used to modify a plant's traits. An illustrative example of trait modification, improved characteristics, by altering expression levels of a particular transcription factor is described further in the Examples and the Sequence Listing.

Arabidopsis as a model system. Arabidopsis thaliana is the object of rapidly growing attention as a model for genetics and metabolism in plants. Arabidopsis has a small genome, and well-documented studies are available. It is easy to grow in large numbers and mutants defining important genetically controlled mechanisms are either available, or can readily be obtained. Various methods to introduce and express isolated homologous genes are available (see Koncz et al., editors, Methods in Arabidopsis Research (1992) World Scientific, New Jersey, in “Preface”). Because of its small size, short life cycle, obligate autogamy and high fertility, Arabidopsis is also a choice organism for the isolation of mutants and studies in morphogenetic and development pathways, and control of these pathways by transcription factors (Koncz (1992) supra, p. 72). A number of studies introducing transcription factors into A. thaliana have demonstrated the utility of this plant for understanding the mechanisms of gene regulation and trait alteration in plants. (See, for example, Koncz supra, and U.S. Pat. No. 6,417,428).

Arabidopsis genes in transgenic plants. Expression of genes that encode transcription factors modify expression of endogenous genes, polynucleotides, and proteins are well known in the art. In addition, transgenic plants comprising isolated polynucleotides encoding transcription factors may also modify expression of endogenous genes, polynucleotides, and proteins. Examples include Peng et al. (1997) et al. Genes and Development 11: 3194-3205, and Peng et al. (1999) Nature 400: 256-261. In addition, many others have demonstrated that an Arabidopsis transcription factor expressed in an exogenous plant species elicits the same or very similar phenotypic response. See, for example, Fu et al. (2001) Plant Cell 13: 1791-1802; Nandi et al. (2000) Curr. Biol. 10: 215-218; Coupland (1995) Nature 377: 482-483; and Weigel and Nilsson (1995) Nature 377: 482-500.

Homologous genes introduced into transgenic plants. Homologous genes that may be derived from any plant, or from any source whether natural, synthetic, semi-synthetic or recombinant, and that share significant sequence identity or similarity to those provided by the present invention, may be introduced into plants, for example, crop plants, to confer desirable or improved traits. Consequently, transgenic plants may be produced that comprise a recombinant expression vector or cassette with a promoter operably linked to one or more sequences homologous to presently disclosed sequences. The promoter may be, for example, a plant or viral promoter.

The invention thus provides for methods for preparing transgenic plants, and for modifying plant traits. These methods include introducing into a plant a recombinant expression vector or cassette comprising a functional promoter operably linked to one or more sequences homologous to presently disclosed sequences. Plants and kits for producing these plants that result from the application of these methods are also encompassed by the present invention.

Transcription factors of interest for the modification of plant traits. Currently, the existence of a series of maturity groups for different latitudes represents a major barrier to the introduction of new valuable traits. Any trait (e.g. disease resistance) has to be bred into each of the different maturity groups separately, a laborious and costly exercise. The availability of a single strain that could be grown at any latitude would therefore greatly increase the potential for introducing new traits to crop species such as soybean and cotton.

More than one transcription factor gene may be introduced into a plant, either by transforming the plant with one or more vectors comprising two or more transcription factors, or by selective breeding of plants to yield hybrid crosses that comprise more than one introduced transcription factor.

Many of the transcription factors listed in the Sequence Listing may be operably linked with a specific promoter that causes the transcription factor to be expressed in response to environmental, tissue-specific or temporal signals. For examples of flower specific promoters, see Kaiser et al. (supra). For examples of other tissue-specific, temporal-specific or inducible promoters, see the above discussion under the heading “Vectors, Promoters, and Expression Systems”.

Antisense and co-suppression. In addition to expression of the nucleic acids of the invention as gene replacement or plant phenotype modification nucleic acids, the nucleic acids are also useful for sense and anti-sense suppression of expression, e.g., to down-regulate expression of a nucleic acid of the invention, e.g., as a further mechanism for modulating plant phenotype. That is, the nucleic acids of the invention, or subsequences or anti-sense sequences thereof, can be used to block expression of naturally occurring homologous nucleic acids. A variety of sense and anti-sense technologies are known in the art, e.g., as set forth in Lichtenstein and Nellen (1997) Antisense Technology: A Practical Approach IRL Press at Oxford University Press, Oxford, U.K. Antisense regulation is also described in Crowley et al. (1985) Cell 43: 633-641; Rosenberg et al. (1985) Nature 313: 703-706; Preiss et al. (1985) Nature 313: 27-32; Melton (1985) Proc. Natl. Acad. Sci. USA 82: 144-148; Izant and Weintraub (1985) Science 229: 345-352; and Kim and Wold (1985) Cell 42: 129-138. Additional methods for antisense regulation are known in the art. Antisense regulation has been used to reduce or inhibit expression of plant genes in, for example in European Patent Publication No. 271988. Antisense RNA may be used to reduce gene expression to produce a visible or biochemical phenotypic change in a plant (Smith et al. (1988) Nature 334: 724-726; Smith et al. (1990) Plant Mol. Biol. 14: 369-379). In general, sense or anti-sense sequences are introduced into a cell, where they are optionally amplified, for example, by transcription. Such sequences include both simple oligonucleotide sequences and catalytic sequences such as ribozymes.

For example, a reduction or elimination of expression (i.e., a “knock-out”) of a transcription factor or transcription factor homolog polypeptide in a transgenic plant, e.g., to modify a plant trait, can be obtained by introducing an antisense construct corresponding to the polypeptide of interest as a cDNA. For antisense suppression, the transcription factor or homolog cDNA is arranged in reverse orientation (with respect to the coding sequence) relative to the promoter sequence in the expression vector. The introduced sequence need not be the full length cDNA or gene, and need not be identical to the cDNA or gene found in the plant type to be transformed. Typically, the antisense sequence need only be capable of hybridizing to the target gene or RNA of interest. Thus, where the introduced sequence is of shorter length, a higher degree of homology to the endogenous transcription factor sequence will be needed for effective antisense suppression. While antisense sequences of various lengths can be utilized, preferably, the introduced antisense sequence in the vector will be at least 30 nucleotides in length, and improved antisense suppression will typically be observed as the length of the antisense sequence increases. Preferably, the length of the antisense sequence in the vector will be greater than 100 nucleotides. Transcription of an antisense construct as described results in the production of RNA molecules that are the reverse complement of mRNA molecules transcribed from the endogenous transcription factor gene in the plant cell.

Suppression of endogenous transcription factor gene expression can also be achieved using a ribozyme. Ribozymes are RNA molecules that possess highly specific endoribonuclease activity. The production and use of ribozymes are disclosed in U.S. Pat. No. 4,987,071 and U.S. Pat. No. 5,543,508. Synthetic ribozyme sequences including antisense RNAs can be used to confer RNA cleaving activity on the antisense RNA, such that endogenous mRNA molecules that hybridize to the antisense RNA are cleaved, which in turn leads to an enhanced antisense inhibition of endogenous gene expression.

Vectors in which RNA encoded by a transcription factor or transcription factor homolog cDNA is over-expressed can also be used to obtain co-suppression of a corresponding endogenous gene, for example, in the manner described in U.S. Pat. No. 5,231,020 to Jorgensen. Such co-suppression (also termed sense suppression) does not require that the entire transcription factor cDNA be introduced into the plant cells, nor does it require that the introduced sequence be exactly identical to the endogenous transcription factor gene of interest. However, as with antisense suppression, the suppressive efficiency will be enhanced as specificity of hybridization is increased, e.g., as the introduced sequence is lengthened, and/or as the sequence similarity between the introduced sequence and the endogenous transcription factor gene is increased.

Vectors expressing an untranslatable form of the transcription factor mRNA (e.g., sequences comprising one or more stop codons or nonsense mutations) can also be used to suppress expression of an endogenous transcription factor, thereby reducing or eliminating its activity and modifying one or more traits. Methods for producing such constructs are described in U.S. Pat. No. 5,583,021. Preferably, such constructs are made by introducing a premature stop codon into the transcription factor gene. Alternatively, a plant trait can be modified by gene silencing using double-strand RNA (Sharp (1999) Genes and Development 13: 139-141). Another method for abolishing the expression of a gene is by insertion mutagenesis using the T-DNA of Agrobacterium tumefaciens. After generating the insertion mutants, the mutants can be screened to identify those containing the insertion in a transcription factor or transcription factor homolog gene. Plants containing a single transgene insertion event at the desired gene can be crossed to generate homozygous plants for the mutation. Such methods are well known to those of skill in the art (See for example Koncz et al. (1992) Methods in Arabidopsis Research, World Scientific Publishing Co. Pte. Ltd., River Edge N.J.).

Suppression of endogenous transcription factor gene expression can also be achieved using RNA interference (RNAi). RNAi is a post-transcriptional, targeted gene-silencing technique that uses double-stranded RNA (dsRNA) to incite degradation of mRNA containing the same sequence as the dsRNA (Constans, (2002) The Scientist 16:36). Small interfering RNAs, or siRNAs are produced in at least two steps: an endogenous ribonuclease cleaves longer dsRNA into shorter, 21-23 nucleotide-long RNAs. The siRNA segments then mediate the degradation of the target mRNA (Zamore, (2001) Nature Struct. Biol., 8:746-50). RNAi has been used for gene function determination in a manner similar to antisense oligonucleotides (Constans, (2002) The Scientist 16:36). Expression vectors that continually express siRNAs in transiently and stably transfected have been engineered to express small hairpin RNAs (shRNAs), which get processed in vivo into siRNAs-like molecules capable of carrying out gene-specific silencing (Brummelkamp et al., (2002) Science 296:550-553, and Paddison, et al. (2002) Genes & Dev. 16:948-958). Post-transcriptional gene silencing by double-stranded RNA is discussed in further detail by Hammond et al. (2001) Nature Rev Gen 2: 110-119, Fire et al. (1998) Nature 391: 806-811 and Timmons and Fire (1998) Nature 395: 854.

Alternatively, a plant phenotype can be altered by eliminating an endogenous gene, such as a transcription factor or transcription factor homolog, e.g., by homologous recombination (Kempin et al. (1997) Nature 389: 802-803).

A plant trait can also be modified by using the Cre-lox system (for example, as described in U.S. Pat. No. 5,658,772). A plant genome can be modified to include first and second lox sites that are then contacted with a Cre recombinase. If the lox sites are in the same orientation, the intervening DNA sequence between the two sites is excised. If the lox sites are in the opposite orientation, the intervening sequence is inverted.

The polynucleotides and polypeptides of this invention can also be expressed in a plant in the absence of an expression cassette by manipulating the activity or expression level of the endogenous gene by other means, such as, for example, by ectopically expressing a gene by T-DNA activation tagging (Ichikawa et al. (1997) Nature 390 698-701; Kakimoto et al. (1996) Science 274: 982-985). This method entails transforming a plant with a gene tag containing multiple transcriptional enhancers and once the tag has inserted into the genome, expression of a flanking gene coding sequence becomes deregulated. In another example, the transcriptional machinery in a plant can be modified so as to increase transcription levels of a polynucleotide of the invention (see, for example, PCT Publications WO 96/06166 and WO 98/53057 that describe the modification of the DNA-binding specificity of zinc finger transcription factor proteins by changing particular amino acids in the DNA-binding motif).

The transgenic plant can also include the machinery necessary for expressing or altering the activity of a polypeptide encoded by an endogenous gene, for example, by altering the phosphorylation state of the polypeptide to maintain it in an activated state.

Transgenic plants (or plant cells, or plant explants, or plant tissues) incorporating the polynucleotides of the invention and/or expressing the polypeptides of the invention can be produced by a variety of well established techniques as described above. Following construction of a vector, most typically an expression cassette, including a polynucleotide, e.g., encoding a transcription factor or transcription factor homolog, of the invention, standard techniques can be used to introduce the polynucleotide into a plant, a plant cell, a plant explant or a plant tissue of interest. Optionally, the plant cell, explant or tissue can be regenerated to produce a transgenic plant.

The plant can be any higher plant, including gymnosperms, monocotyledonous and dicotyledenous plants. Suitable protocols are available for Leguminosae (alfalfa, soybean, clover, etc.), Umbelliferae (carrot, celery, parsnip), Cruciferae (cabbage, radish, rapeseed, broccoli, etc.), Curcurbitaceae (melons and cucumber), Gramineae (wheat, corn, rice, barley, millet, etc.), Solanaceae (potato, tomato, tobacco, peppers, etc.), and various other crops. See protocols described in Ammirato et al., Editors, (1984) Handbook of Plant Cell Culture—Crop Species, Macmillan Publ. Co., New York N.Y.; Shimamoto et al. (1989) Nature 338: 274-276; Fromm et al. (1990) Bio/Technol. 8: 833-839; and Vasil et al. (1990) Bio/Technol. 8: 429-434.

Transformation and regeneration of both monocotyledonous and dicotyledonous plant cells is now routine, and the selection of the most appropriate transformation technique will be determined by the practitioner. The choice of method will vary with the type of plant to be transformed; those skilled in the art will recognize the suitability of particular methods for given plant types. Suitable methods can include, but are not limited to: electroporation of plant protoplasts; liposome-mediated transformation; polyethylene glycol (PEG) mediated transformation; transformation using viruses; micro-injection of plant cells; micro-projectile bombardment of plant cells; vacuum infiltration; and Agrobacterium tumefaciens-mediated transformation. Transformation means introducing a nucleotide sequence into a plant in a manner to cause stable or transient expression of the sequence.

Successful examples of the modification of plant characteristics by transformation with cloned sequences that serve to illustrate the current knowledge in this field of technology, and which are herein incorporated by reference, include: U.S. Pat. Nos. 5,571,706; 5,677,175; 5,510,471; 5,750,386; 5,597,945; 5,589,615; 5,750,871; 5,268,526; 5,780,708; 5,538,880; 5,773,269; 5,736,369 and 5,610,042.

Following transformation, plants are preferably selected using a dominant selectable marker incorporated into the transformation vector. Typically, such a marker will confer antibiotic or herbicide resistance on the transformed plants, and selection of transformants can be accomplished by exposing the plants to appropriate concentrations of the antibiotic or herbicide.

After transformed plants are selected and grown to maturity, those plants showing a modified trait are identified. The modified trait can be any of those traits described above. Additionally, to confirm that the modified trait is due to changes in expression levels or activity of the polypeptide or polynucleotide of the invention can be determined by analyzing mRNA expression using Northern blots, RT-PCR or microarrays, or protein expression using immunoblots or Western blots or gel shift assays.

Integrated Systems—Sequence Identity. Additionally, the present invention may be an integrated system, computer or computer readable medium that comprises an instruction set for determining the identity of one or more sequences in a database. The instruction set can also be used to generate or identify sequences that meet any specified criteria. Furthermore, the instruction set may be used to associate or link certain functional benefits, such improved characteristics, with one or more identified sequence.

For example, the instruction set can include, e.g., a sequence comparison or other alignment program, e.g., an available program such as, for example, the Wisconsin Package Version 10.0, such as BLAST, FASTA, PILEUP, FINDPATTERNS, or the like (GCG, Madison, Wis.). Public sequence databases such as GenBank, EMBL, Swiss-Prot and PIR, or private sequence databases such as PHYTOSEQ sequence database (Incyte Genomics, Wilmington, Del.) can be searched.

Alignment of sequences for comparison can be conducted by the local homology algorithm of Smith and Waterman (1981) Adv. Appl. Math. 2: 482-489, by the homology alignment algorithm of Needleman and Wunsch (1970) J. Mol. Biol. 48: 443-453, by the search for similarity method of Pearson and Lipman (1988) Proc. Natl. Acad. Sci. USA 85: 2444-2448, by computerized implementations of these algorithms. After alignment, sequence comparisons between two (or more) polynucleotides or polypeptides are typically performed by comparing sequences of the two sequences over a comparison window to identify and compare local regions of sequence similarity. The comparison window can be a segment of at least about 20 contiguous positions, usually about 50 to about 200, more usually about 100 to about 150 contiguous positions. A description of the method is provided in Ausubel (2000) supra.

A variety of methods for determining sequence relationships can be used, including manual alignment and computer assisted sequence alignment and analysis. This later approach is a preferred approach in the present invention, due to the increased throughput afforded by computer assisted methods. As noted above, a variety of computer programs for performing sequence alignment are available, or can be produced by one of skill in the art.

One example algorithm that is suitable for determining percent sequence identity and sequence similarity is the BLAST algorithm, which is described in Altschul et al. (1990) supra. Software for performing BLAST analyses is publicly available, e.g., through the National Library of Medicine's National Center for Biotechnology Information (ncbi.nln.nih; see at world wide web (www) National Institutes of Health US government (gov) website). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215: 403-410). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff and Henikoff (1992) Proc. Natl. Acad. Sci. USA 89: 10915-10919). Unless otherwise indicated, “sequence identity” refers to the percent sequence identity generated from a tblastx analysis using the NCBI version of the algorithm at the default settings using gapped alignments with the filter “off” (NIH NLM NCBI website at ncbi.nlm.nih, supra).

In addition to calculating percent sequence identity, the BLAST algorithm also performs a statistical analysis of the similarity between two sequences (see, for example, Karlin and Altschul (1993) Proc. Natl. Acad. Sci. USA 90: 5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence (and, therefore, in this context, homologous) if the smallest sum probability in a comparison of the test nucleic acid to the reference nucleic acid is less than about 0.1, or less than about 0.01, and or even less than about 0.001. An additional example of a useful sequence alignment algorithm is PILEUP. PILEUP creates a multiple sequence alignment from a group of related sequences using progressive, pairwise alignments. The program can align, for example, up to 300 sequences of a maximum length of 5,000 letters.

The integrated system, or computer typically includes a user input interface allowing a user to selectively view one or more sequence records corresponding to the one or more character strings, as well as an instruction set that aligns the one or more character strings with each other or with an additional character string to identify one or more region of sequence similarity. The system may include a link of one or more character strings with a particular phenotype or gene function. Typically, the system includes a user readable output element that displays an alignment produced by the alignment instruction set.

The methods of this invention can be implemented in a localized or distributed computing environment. In a distributed environment, the methods may implemented on a single computer comprising multiple processors or on a multiplicity of computers. The computers can be linked, e.g. through a common bus, but more preferably the computer(s) are nodes on a network. The network can be a generalized or a dedicated local or wide-area network and, in certain preferred embodiments, the computers may be components of an intra-net or an internet.

Thus, the invention provides methods for identifying a sequence similar or homologous to one or more polynucleotides as noted herein, or one or more target polypeptides encoded by the polynucleotides, or otherwise noted herein and may include linking or associating a given plant phenotype or gene function with a sequence. In the methods, a sequence database is provided (locally or across an inter or intra net) and a query is made against the sequence database using the relevant sequences herein and associated plant phenotypes or gene functions.

Any sequence herein can be entered into the database, before or after querying the database. This provides for both expansion of the database and, if done before the querying step, for insertion of control sequences into the database. The control sequences can be detected by the query to ensure the general integrity of both the database and the query. As noted, the query can be performed using a web browser based interface. For example, the database can be a centralized public database such as those noted herein, and the querying can be done from a remote terminal or computer across an internet or intranet.

Any sequence herein can be used to identify a similar, homologous, paralogous, or orthologous sequence in another plant. This provides means for identifying endogenous sequences in other plants that may be useful to alter a trait of progeny plants, which results from crossing two plants of different strain. For example, sequences that encode an ortholog of any of the sequences herein that naturally occur in a plant with a desired trait can be identified using the sequences disclosed herein. The plant is then crossed with a second plant of the same species but which does not have the desired trait to produce progeny that can then be used in further crossing experiments to produce the desired trait in the second plant. Therefore the resulting progeny plant contains no transgenes; expression of the endogenous sequence may also be regulated by treatment with a particular chemical or other means, such as EMR. Some examples of such compounds well known in the art include: ethylene; cytokinins; phenolic compounds, which stimulate the transcription of the genes needed for infection; specific monosaccharides and acidic environments that potentiate vir gene induction; acidic polysaccharides that induce one or more chromosomal genes; and opines; other mechanisms include light or dark treatment (for a review of examples of such treatments, see, Winans (1992) Microbiol. Rev. 56: 12-31; Eyal et al. (1992) Plant Mol. Biol. 19: 589-599; Chrispeels et al. (2000) Plant Mol. Biol. 42: 279-290; Piazza et al. (2002) Plant Physiol. 128: 1077-1086).

Table 6 lists a summary of homologous sequences identified using BLAST (tblastx program). The first column shows the orthologous or homologous polynucleotide GenBank Accession Number (Test Sequence ID), the second column shows the calculated probability value that the sequence identity is due to chance (Smallest Sum Probability), the third column shows the plant species from which the test sequence was isolated (Test Sequence Species), and the fourth column shows the orthologous or homologous test sequence GenBank annotation (Test Sequence GenBank Annotation).

TABLE 6
Sequences orthologous to G28 identified using BLAST

| | Smallest Sum | | Test Sequence GenBank |
|---|---|---|---|
| Test Sequence ID | Probability | Test Sequence Species | Annotation |
| AF245119 | 2.00E−72 | Mesembryanthemum crystallinum | AP2-related transcription fac |
| BQ165291 | 1.00E−68 | Medicago truncatula | EST611160 KVKC Medicago |
| | | | truncatula cDNA |
| AB016264 | 1.00E−57 | Nicotiana sylvestris | nserf2 gene for ethylene- |
| | | | responsive el |
| TOBBY4D | 2.00E−57 | Nicotiana tabacum | Tobacco mRNA for EREBP-2, |
| | | | complete cds. |
| BQ047502 | 2.00E−57 | Solanum tuberosum | EST596620 P. infestans- |
| | | | challenged potato |
| LEU89255 | 2.00E−56 | Lycopersicon esculentum | DNA-binding protein Pti4 |
| | | | mRNA, comp |
| BH454277 | 2.00E−54 | Brassica oleracea | BOGSI45TR BOGS Brassica |
| | | | oleracea genomic |
| BE449392 | 1.00E−53 | Lycopersicon hirsutum | EST356151 L. hirsutum |
| | | | trichome, Corne |
| AB035270 | 2.00E−50 | Matricaria chamomilla | McEREBP1 mRNA for |
| | | | ethylene-responsive |
| AW233956 | 5.00E−50 | Glycine max | sf32e02.y1 Gm-c1028 Glycine |
| | | | max cDNA clone GENO |
| gi7528276 | 6.10E−71 | Mesembryanthemum crystallinum | AP2-related transcription f |
| gi8809571 | 3.30E−56 | Nicotiana sylvestris | ethylene-responsive element |
| | | | binding |
| gi3342211 | 4.20E−56 | Lycopersicon esculentum | Pti4. |
| gi1208498 | 8.70E−56 | Nicotiana tabacum | EREBP-2. |
| gi14140141 | 4.20E−49 | Oryza sativa | putative AP2-related |
| | | | transcription factor. |
| gi17385636 | 3.00E−46 | Matricaria chamomilla | ethylene-responsive element |
| | | | binding |
| gi21304712 | 2.90E−31 | Glycine max | ethylene-responsive element |
| | | | binding protein 1 |
| gi15623863 | 5.60E−29 | Oryza sativa (japonica cultivar- | contains EST˜hypot |
| | | group) | |
| gi8980313 | 1.20E−26 | Catharanthus roseus | AP2-domain DNA-binding |
| | | | protein. |
| gi4099921 | 3.10E−21 | Stylosanthes hamata | EREBP-3 homolog. |

Molecular Modeling

Another means that may be used to confirm the utility and function of transcription factor sequences that are orthologous or paralogous to presently disclosed transcription factors is through the use of molecular modeling software. Molecular modeling is routinely used to predict polypeptide structure, and a variety of protein structure modeling programs, such as “Insight II” (Accelrys, Inc.) are commercially available for this purpose. Modeling can thus be used to predict which residues of a polypeptide can be changed without altering function (Crameri et al. (2003) U.S. Pat. No. 6,521,453). Thus, polypeptides that are sequentially similar can be shown to have a high likelihood of similar function by their structural similarity, which may, for example, be established by comparison of regions of superstructure. The relative tendencies of amino acids to form regions of superstructure (for example, α-helixes and β-sheets) are well established. For example, O'Neil et al. ((1990) Science 250: 646-651) have discussed in detail the helix forming tendencies of amino acids. Tables of relative structure forming activity for amino acids can be used as substitution tables to predict which residues can be functionally substituted in a given region, for example, in DNA-binding domains of known transcription factors and equivalogs. Homologs that are likely to be functionally similar can then be identified.

Of particular interest is the structure of a transcription factor in the region of its conserved domains, such as those identified in FIGS. 3A-3B (Motif Y) and FIGS. 3D-3E (AP2 domains). Structural analyses may be performed by comparing the structure of the known transcription factor around its conserved domain with those of orthologs and paralogs. Analysis of a number of polypeptides within a transcription factor group or clade, including the functionally or sequentially similar polypeptides provided in the Sequence Listing, may also provide an understanding of structural elements required to regulate transcription within a given family.

### EXAMPLES

It is to be understood that this invention is not limited to the particular materials and methods described. Although particular embodiments are described, equivalent embodiments may be used to practice the invention. The described embodiments are not intended to limit the scope of the invention, which is limited only by the appended claims. The examples below are provided to enable the subject invention and are not included for the purpose of limiting the invention.

The invention, now being generally described, will be more readily understood by reference to the following examples, which are included merely for purposes of illustration of certain aspects and embodiments of the present invention and are not intended to limit the invention. It will be recognized by one of skill in the art that a transcription factor associated with a particular first trait may be associated with at least one other, unrelated and inherent second trait that was not predicted by the first trait.

### Example I

### Full Length Gene Identification and Cloning

Putative transcription factor sequences (genomic or ESTs) related to known transcription factors were identified in the Arabidopsis thaliana GenBank database using the tblastn sequence analysis program using default parameters and a P-value cutoff threshold of −4 or −5 or lower, depending on the length of the query sequence. Putative transcription factor sequence hits were then screened to identify those containing particular sequence strings. If the sequence hits contained such sequence strings, the sequences were confirmed as transcription factors.

Alternatively, Arabidopsis thaliana cDNA libraries derived from different tissues or treatments, or genomic libraries were screened to identify novel members of a transcription family using a low stringency hybridization approach. Probes were synthesized using gene specific primers in a standard PCR reaction (annealing temperature 60° C.) and labeled with ³²P dCTP using the High Prime DNA Labeling Kit (Boehringer Mannheim Corp. (now Roche Diagnostics Corp., Indianapolis, Ind.). Purified radiolabelled probes were added to filters immersed in Church hybridization medium (0.5 M NaPO₄ pH 7.0, 7% SDS, 1% w/v bovine serum albumin) and hybridized overnight at 60° C. with shaking. Filters were washed two times for 45 to 60 minutes with 1×SCC, 1% SDS at 60° C.

To identify additional sequence 5′ or 3′ of a partial cDNA sequence in a cDNA library, 5′ and 3′ rapid amplification of cDNA ends (RACE) was performed using the MARATHON cDNA amplification kit (Clontech, Palo Alto, Calif.). Generally, the method entailed first isolating poly(A) mRNA, performing first and second strand cDNA synthesis to generate double stranded cDNA, blunting cDNA ends, followed by ligation of the MARATHON Adaptor to the cDNA to form a library of adaptor-ligated ds cDNA.

Gene-specific primers were designed to be used along with adaptor specific primers for both 5′ and 3′ RACE reactions. Nested primers, rather than single primers, were used to increase PCR specificity. Using 5′ and 3′ RACE reactions, 5′ and 3′ RACE fragments were obtained, sequenced and cloned. The process can be repeated until 5′ and 3′ ends of the full-length gene were identified. Then the full-length cDNA was generated by PCR using primers specific to 5′ and 3′ ends of the gene by end-to-end PCR.

### Example II

### Construction of Expression Vectors

The sequence was amplified from a genomic or cDNA library using primers specific to sequences upstream and downstream of the coding region. The expression vector was pMEN20 or pMEN65, which are both derived from pMON316 (Sanders et al. (1987) Nucleic Acids Res. 15:1543-1558) and contain the CaMV 35S promoter to express transgenes. To clone the sequence into the vector, both pMEN20 and the amplified DNA fragment were digested separately with SalI and NotI restriction enzymes at 37° C. for 2 hours. The digestion products were subject to electrophoresis in a 0.8% agarose gel and visualized by ethidium bromide staining. The DNA fragments containing the sequence and the linearized plasmid were excised and purified by using a QIAQUICK gel extraction kit (Qiagen, Valencia, Calif.). The fragments of interest were ligated at a ratio of 3:1 (vector to insert). Ligation reactions using T4 DNA ligase (New England Biolabs, Beverly Mass.) were carried out at 16° C. for 16 hours. The ligated DNAs were transformed into competent cells of the E. coli strain DH5alpha by using the heat shock method. The transformations were plated on LB plates containing 50 mg/l kanamycin (Sigma Chemical Co. St. Louis Mo.). Individual colonies were grown overnight in five milliliters of LB broth containing 50 mg/l kanamycin at 37° C. Plasmid DNA was purified by using Qiaquick Mini Prep kits (Qiagen, Valencia Calif.).

### Example III

### Transformation of Agrobacterium with the Expression Vector

After the plasmid vector containing the gene was constructed, the vector was used to transform Agrobacterium tumefaciens cells expressing the gene products. The stock of Agrobacterium tumefaciens cells for transformation were made as described by Nagel et al. (1990) FEMS Microbiol Letts. 67: 325-328. Agrobacterium strain ABI was grown in 250 ml LB medium (Sigma) overnight at 28° C. with shaking until an absorbance over 1 cm at 600 nm (A₆₀₀) of 0.5-1.0 was reached. Cells were harvested by centrifugation at 4,000×g for 15 minutes at 4° C. Cells were then resuspended in 250 μl chilled buffer (1 mM HEPES, pH adjusted to 7.0 with KOH). Cells were centrifuged again as described above and resuspended in 125 μl chilled buffer. Cells were then centrifuged and resuspended two more times in the same HEPES buffer as described above at a volume of 100 μl and 750 μl, respectively. Resuspended cells were then distributed into 40 μl aliquots, quickly frozen in liquid nitrogen, and stored at −80° C.

Agrobacterium cells were transformed with plasmids prepared as described above following the protocol described by Nagel et al. (1990) supra. For each DNA construct to be transformed, 50-100 ng DNA (generally resuspended in 10 mM Tris-HCl, 1 mM EDTA, pH 8.0) was mixed with 40 μl of Agrobacterium cells. The DNA/cell mixture was then transferred to a chilled cuvette with a 2 mm electrode gap and subject to a 2.5 kV charge dissipated at 25 μF and 200 μF using a Gene Pulser II apparatus (Bio-Rad, Hercules, Calif.). After electroporation, cells were immediately resuspended in 1.0 ml LB and allowed to recover without antibiotic selection for 2-4 hours at 28° C. in a shaking incubator. After recovery, cells were plated onto selective medium of LB broth containing 100 μg/ml spectinomycin (Sigma) and incubated for 24-48 hours at 28° C. Single colonies were then picked and inoculated in fresh medium. The presence of the plasmid construct was verified by PCR amplification and sequence analysis.

### Example IV

### Transformation of Arabidopsis Plants

After transformation of Agrobacterium tumefaciens with plasmid vectors containing the gene, single Agrobacterium colonies were identified, propagated, and used to transform Arabidopsis plants. Briefly, 500 ml cultures of LB medium containing 50 mg/l kanamycin were inoculated with the colonies and grown at 28° C. with shaking for 2 days until an optical absorbance at 600 nm wavelength over 1 cm (A₆₀₀) of >2.0 is reached. Cells were then harvested by centrifugation at 4,000×g for 10 minutes, and resuspended in infiltration medium (½× Murashige and Skoog salts (Sigma), 1× Gamborg's B-5 vitamins (Sigma), 5.0% (w/v) sucrose (Sigma), 0.044 μM benzylamino purine (Sigma), 200 μl/l Silwet L-77 (Lehle Seeds) until an A₆₀₀ of 0.8 was reached.

Prior to transformation, Arabidopsis thaliana seeds (ecotype Columbia) were sown at a density of about 10 plants per 4″ pot onto Pro-Mix BX potting medium (Hummert International) covered with fiberglass mesh (18 mm×16 mm). Plants were grown under continuous illumination (50-75 μE/m²/second) at 22-23° C. with 65-70% relative humidity. After about 4 weeks, primary inflorescence stems (bolts) are cut off to encourage growth of multiple secondary bolts. After flowering of the mature secondary bolts, plants were prepared for transformation by removal of all siliques and opened flowers.

The pots were then immersed upside down in the mixture of Agrobacterium infiltration medium as described above for 30 seconds, and placed on their sides to allow draining into a 1′×2′ flat surface covered with plastic wrap. After 24 hours, the plastic wrap was removed and pots are turned upright. The immersion procedure was repeated one week later, for a total of two immersions per pot. Seeds were then collected from each transformation pot and analyzed following the protocol described below.

### Example V

### Identification of Arabidopsis Primary Transformants

Seeds collected from the transformation pots were sterilized essentially as follows. Seeds were dispersed into in a solution containing 0.1% (v/v) Triton X-100 (Sigma) and sterile water and washed by shaking the suspension for 20 minutes. The wash solution was then drained and replaced with fresh wash solution to wash the seeds for 20 minutes with shaking. After removal of the ethanol/detergent solution, a solution containing 0.1% (v/v) Triton X-100 and 30% (v/v) bleach (CLOROX; Clorox Corp. Oakland Calif.) was added to the seeds, and the suspension was shaken for 10 minutes. After removal of the bleach/detergent solution, seeds were then washed five times in sterile distilled water. The seeds were stored in the last wash water at 4° C. for 2 days in the dark before being plated onto antibiotic selection medium (1× Murashige and Skoog salts (pH adjusted to 5.7 with 1M KOH), 1× Gamborg's B-5 vitamins, 0.9% phytagar (Life Technologies), and 50 mg/l kanamycin). Seeds were germinated under continuous illumination (50-75 μE/m²/second) at 22-23° C. After 7-10 days of growth under these conditions, kanamycin resistant primary transformants (T₁ generation) were visible and obtained. These seedlings were transferred first to fresh selection plates where the seedlings continued to grow for 3-5 more days, and then to soil (Pro-Mix BX potting medium).

Primary transformants were crossed and progeny seeds (T₂) collected; kanamycin resistant seedlings were selected and analyzed. The expression levels of the recombinant polynucleotides in the transformants varies from about a 5% expression level increase to a least a 100% expression level increase. Similar observations are made with respect to polypeptide level expression.

### Example VI

### Identification of Arabidopsis Plants with Transcription Factor Gene Knockouts

The screening of insertion mutagenized Arabidopsis collections for null mutants in a known target gene was essentially as described in Krysan et al. (1999) Plant Cell 11: 2283-2290. Briefly, gene-specific primers, nested by 5-250 base pairs to each other, were designed from the 5′ and 3′ regions of a known target gene. Similarly, nested sets of primers were also created specific to each of the T-DNA or transposon ends (the “right” and “left” borders). All possible combinations of gene specific and T-DNA/transposon primers were used to detect by PCR an insertion event within or close to the target gene. The amplified DNA fragments were then sequenced, which allows the precise determination of the T-DNA/transposon insertion point relative to the target gene. Insertion events within the coding or intervening sequence of the genes were deconvoluted from a pool comprising a plurality of insertion events to a single unique mutant plant for functional characterization. The method is described in more detail in Yu and Adam, U.S. application Ser. No. 09/177,733 filed Oct. 23, 1998.

### Example VII

### Identification of Modified Phenotypes in Overexpressing or Knockout Plants

Experiments were performed to identify those transformants or knockouts that exhibited an improved pathogen tolerance. For such studies, the transformants were exposed to biotropic fungal pathogens, such as Erysiphe orontii, and necrotropic fungal pathogens, such as Fusarium oxysporum. Fusarium oxysporum isolates cause vascular wilts and damping off of various annual vegetables, perennials and weeds (Mauch-Mani and Slusarenko (1994) Molec Plant-Microbe Interact. 7: 378-383). For Fusarium oxysporum experiments, plants were grown on Petri dishes and sprayed with a fresh spore suspension of F. oxysporum. The spore suspension was prepared as follows: a plug of fungal hyphae from a plate culture was placed on a fresh potato dextrose agar plate and allowed to spread for one week. Five ml sterile water was added to the plate, swirled, and pipetted into 50 ml Armstrong Fusarium medium. Spores were grown overnight in Fusarium medium and then sprayed onto plants using a Preval paint sprayer. Plant tissue was harvested and frozen in liquid nitrogen 48 hours post-infection.

Erysiphe orontii is a causal agent of powdery mildew. For Erysiphe orontii experiments, plants were grown approximately four weeks in a greenhouse under 12 hour light (20° C., about 30% relative humidity (rh)). Individual leaves were infected with E. orontii spores from infected plants using a camel's hair brush, and the plants were transferred to a Percival growth chamber (20° C., 80% rh.). Plant tissue was harvested and frozen in liquid nitrogen seven days post-infection.

Botrytis cinerea is a necrotrophic pathogen. Botrytis cinerea was grown on potato dextrose agar under 12 hour light (20° C., about 30% relative humidity (rh)). A spore culture was made by spreading 10 ml of sterile water on the fungus plate, swirling and transferring spores to 10 ml of sterile water. The spore inoculum (approx. 105 spores/ml) was then used to spray 10 day-old seedlings grown under sterile conditions on MS (minus sucrose) media. Symptoms were evaluated every day up to approximately 1 week.

Sclerotinia sclerotiorum hyphal cultures were grown in potato dextrose broth. One gram of hyphae was ground, filtered, spun down and resuspended in sterile water. A 1:10 dilution was used to spray 10 day-old seedlings grown aseptically under a 12 hour light/dark regime on MS (minus sucrose) media. Symptoms were evaluated every day up to approximately 1 week.

Pseudomonas syringae pv maculicola (Psm) strain 4326 and pv maculicola strain 4326 was inoculated by hand at two doses. Two inoculation doses allowed the differentiation between plants with enhanced susceptibility and plants with enhanced resistance to the pathogen. Plants were grown for three weeks in the greenhouse, then transferred to the growth chamber for the remainder of their growth. Psm ES4326 was hand inoculated with 1 ml syringe on three fully-expanded leaves per plant (4½ wk old), using at least nine plants per overexpressing line at two inoculation doses, OD=0.005 and OD=0.0005. Disease scoring was performed three post-inoculation by evaluating the plants and leaves simultaneously.

Expression patterns of the pathogen-induced genes (such as defense genes) was also monitored by microarray experiments. In these experiments, cDNAs were generated by PCR and resuspended at a final concentration of about 100 ng/μl in 3×SSC or 150 mM Na-phosphate (Eisen and Brown (1999) Methods Enzymol. 303: 179-205). The cDNAs were spotted on microscope glass slides coated with polylysine. The prepared cDNAs were aliquoted into 384 well plates and spotted on the slides using, for example, an x-y-z gantry (OmniGrid; GeneMachines Menlo Park, Calif.) outfitted with quill type pins (Telechem International, Sunnyvale, Calif.). After spotting, the arrays were cured for a minimum of one week at room temperature, rehydrated and blocked following the protocol of Eisen and Brown (Eisen and Brown (1999) supra).

Sample total RNA (10 μg) samples were labeled using fluorescent Cy3 and Cy5 dyes. Labeled samples were resuspended in 4×SSC/0.03% SDS/4 μg salmon sperm DNA/2 μg tRNA/50 mM Na-pyrophosphate, heated for 95° C. for 2.5 minutes, spun down and placed on the array. The array was covered with a glass coverslip and placed in a sealed chamber. The chamber was kept in a water bath at 62° C. overnight. The arrays were washed as described (Eisen and Brown (1999) supra) and scanned on a General Scanning 3000 laser scanner. The resulting files were quantified with IMAGENE software (BioDiscovery, Los Angeles Calif.).

Reverse transcriptase PCR or RT-PCR experiments may be performed to identify those genes induced after exposure to biotropic fungal pathogens, such as Erysiphe orontii, necrotropic fungal pathogens, such as Fusarium oxysporum, bacteria, viruses and salicylic acid, the latter being involved in a nonspecific resistance response in Arabidopsis thaliana. Generally, the gene expression patterns from ground plant leaf tissue was examined. RT-PCR was conducted using gene specific primers within the coding region for each sequence identified. The primers were designed near the 3′ region of each DNA binding sequence initially identified.

Total RNA from ground leaf tissues was isolated using the CTAB extraction protocol. Once extracted total RNA was normalized in concentration across all the tissue types to ensure that the PCR reaction for each tissue received the same amount of cDNA template using the 28S band as reference. Poly(A+) RNA was purified using a modified protocol from the Qiagen OLIGOTEX purification kit batch protocol. cDNA was synthesized using standard protocols. After the first strand cDNA synthesis, primers for Actin 2 were used to normalize the concentration of cDNA across the tissue types. Actin 2 is found to be constitutively expressed in fairly equal levels across the tissue types being investigated.

cDNA template was mixed with corresponding primers and Taq DNA polymerase. Each reaction consisted of 0.2 μl cDNA template, 2 μl 10× Tricine buffer, 2 μl 10× Tricine buffer and 16.8 μl water, 0.05 μl Primer 1, 0.05 μl, Primer 2, 0.3 μl Taq DNA polymerase and 8.6 μl water.

The 96 well plate was covered with microfilm and set in the thermocycler to start the reaction cycle. A typical reaction cycle consisted of the following steps:

Step 1: 93° C. for 3 minutes;

Step 2: 93° C. for 30 seconds;

Step 3: 65° C. for 1 minute;

Step 4: 72° C. for 2 minutes;

Steps 2, 3 and 4 are repeated for 28 cycles;

Step 5: 72° C. for 5 minutes; and

Step 6. 4° C.

To amplify more products, for example, to identify genes that have very low expression, additional steps may be performed: The following method illustrates a method that may be used in this regard. The PCR plate is placed back in the thermocycler for eight more cycles of steps 2-4.

Step 2. 93° C. for 30 seconds;

Step 3. 65° C. for 1 minute;

Step 4. 72° C. for 2 minutes, repeated for 8 cycles; and

Step 5. 4° C.

Eight microliters of PCR product and 1.5 μl of loading dye are loaded on a 1.2% agarose gel for analysis after 28 cycles and 36 cycles. Expression levels of specific transcripts are considered low if they were only detectable after 36 cycles of PCR. Expression levels are considered medium or high depending on the levels of transcript compared with observed transcript levels for an internal control such as actin2. Transcript levels are determined in repeat experiments and compared to transcript levels in control (e.g., non-transformed) plants.

Modified phenotypes observed for particular overexpressor or knockout plants may include increased or decreased disease tolerance or resistance. For a particular overexpressor that shows a less beneficial characteristic such as reduced disease resistance or tolerance, it may be more useful to select a plant with a decreased expression of the particular transcription factor. For a particular knockout that shows a beneficial characteristic, such as increased disease resistance or tolerance, it may be more useful to select a plant with an increased expression of the particular transcription factor.

The transcription factor sequences of the Sequence Listing, or those in the present Tables or Figures, and their equivalogs, can be used to prepare transgenic plants and plants with altered traits. The specific transgenic plants listed below are produced from the sequences of the Sequence Listing, as noted. The Sequence Listing and Tables 1, 2, 6 and 7 provide exemplary polynucleotide and polypeptide sequences of the invention.

### Example VIII

### Description and Overexpression of G28 (Polynucleotide and Polypeptide SEQ ID NO: 1 and 2) and Production of Disease Tolerance or Resistance in Plants

This example provides experimental evidence for the disease tolerance or resistance controlled by the transcription factor polypeptides and polypeptides of the invention, including resistance or tolerance to multiple pathogens provided by G28 and its equivalogs.

Among the goals of these studies was to determine whether altering the expression of G28 or its equivalogs (including those listed in the Sequence Listing) in transgenic plants could confer a significant improvement in pathogen tolerance or resistance. This may be determined by empirical observations of plants that overexpressed G28 or equivalogs after challenge with pathogenic organisms, as compared to control plants similarly treated, as well as by gene expression analyses of these plants for the purpose of demonstrating the expression of direct and indirect pathway targets by G28. These targets generally include specific plant disease resistance genes, including, by way of example but not limitation, genes encoding chitinases, glucanases, enzymes of phytoalexin biosynthesis, defensins, enzymes of lignin biosynthesis, anti-oxidant activities (e.g., glutathione-S-transferases). The pathway targets may be instrumental in a defense response involving localized programmed cell death of infected host cells (the “hypersensitive response”), the accumulation of anti-pathogenic compounds, and cell-wall reinforcement. The hypersensitive response subsequently leads to systemic induction of defense pathways that prevents further infection in a systemic acquired resistance (SAR; Dong (1998) Curr. Opin. Plant Biol. 1: 316-323). SAR is typically effective against a wide variety of pathogen types and can be characterized as an induced broad-spectrum resistance or tolerance.

In a preferred embodiment, overexpression of G28 or an equivalog leads to SAR, i.e., broad-spectrum resistance or tolerance, by induction of multiple direct and indirect pathway targets.

Published Information. Arabidopsis tdr G28 corresponds to AtERF1 (GenBank accession number AB008103; Fujimoto et al. (2000) supra). G28 appears as gene AT4g17500 in the annotated sequence of Arabidopsis chromosome 4 (AL161546.2).

AtERF1 has been shown to have GCC-box binding activity; some defense-related genes that were induced by ethylene were found to contain a short cis-acting element known as the GCC-box: AGCCGCC (Ohme-Takagi and Shinshi (1990) supra). Using transient assays in Arabidopsis leaves, AtERF1 was found to be able to act as a GCC-box sequence-specific transactivator (Fujimoto et al. (2000) supra).

As noted above, ATERF1 expression has been described to be induced by ethylene (two- to three-fold increase in AtERF1 transcript levels 12 hours after ethylene treatment; Fujimoto et al. (2000) supra). In the ein2 mutant, the expression of AtERF1 was not induced by ethylene, suggesting that the ethylene induction of AtERF1 is regulated under the ethylene signaling pathway (Fujimoto et al. (2000) supra). AtERF1 expression was also induced by wounding, but not by other abiotic stresses (such as cold, salinity, or drought; Fujimoto et al. (2000) supra).

It has been suggested that AtERFs, in general, may act as transcription factors for stress-responsive genes, and that the GCC-box may act as a cis-regulatory element for biotic and abiotic stress signal transduction in addition to its role as an ethylene responsive element (ERE; Fujimoto et al. (2000) supra), but there are no data available on the physiological functions of AtERF1.

Experimental Observations, Disease Resistance. G28 is expressed at higher levels when wild type Arabidopsis plants are inoculated with Erysiphe, Fusarium, or treated with salicylic acid, compared with expression levels of G28 in control untreated samples.

A full length G28 cDNA under the control of the CaMV 35S promoter was transformed into wild-type Arabidopsis plants. Twenty independent transgenic T1 lines were planted and nine of those T1 plants were monitored for the expression of the transgene by RT-PCR. The three highest G28 over-expressing lines were carried to the next generation and scored for disease resistance. To ensure that there was no co-suppression in the generation in which the assays were being performed, the expression of G28 from the transgene was monitored by RT-PCR. A high level of G28 induction was observed in this generation and it was concluded that there was not a high level of cosuppression. When three 35S::G28 lines, G28-10, -11 and -15, were tested for resistance to E. orontii, B. cinerea, and S. sclerotiorum, all three lines exhibited enhanced resistance. The G28-15 and G28-11 lines behaved similarly in all the assays and exhibited phenotypes that were much stronger than line G28-10 as measured by disease severity ratings. This was consistent with results from B. cinerea and S. sclerotiorum assays on the same plant lines grown and assayed in tissue culture. Importantly, G28 overexpression conferred increased resistance to pathogens with very different modes of infection, a surprising result. E. orontii is a biotrophic pathogen whereas the other two are necrotrophic. Because it is known that different defense-related signal transduction pathways are activated in response to different pathogen types (Maleck et al. (1999) Trends Plant Sci. 4: 215-219; Pieterse et al. (1999) Trends Plant Sci. 4: 52-58), these results were unexpected and suggest that G28 is a central player in activating multiple resistance mechanisms. This is the reason that G28 transgenic plants were given high priority for further analysis.

As expected for a transcription factor involved in plant defense responses, RT-PCR analysis showed that G28 is expressed in a variety of Arabidopsis tissues (predominantly in shoot, root, rosette, cauline, and germinating seed) and under several disease-related conditions. Importantly, as shown by real-time PCR analysis, G28 appears to be involved in defense response pathways, since its transcription was activated in response to the defense-related hormones jasmonic acid and salicylic acid as well as the fungal pathogen Botrytis cinerea. G28 was previously shown to be induced by ethylene (Fujimoto et al. (2000) supra) and was confirmed experimentally using real-time PCR. The pathogenesis related genes PR1 and PDF1.2 were used as controls for this experiment.

PR1 is a known marker of systemic acquired resistance and is salicylic acid-inducible, and PDF1.2 is the best-characterized gene that is induced by jasmonic acid, ethylene and several necrotrophic fungal pathogens (Maleck et al. (1999) supra; Pieterse et al. (1999) supra). PR1 and PDF1.2 induction were consistent with expectations and showed a steady increase following the appropriate treatments. G28 induction by salicylic acid, 1-aminocyclopropane-1-carboxylic acid (ACC) and jasmonic acid occurred within two hours of treatment and was transient even though the treatment continued throughout the experimental time-course. On the other hand, G28 induction by B. cinerea occurred within two hours of fungal treatment and continued to rise throughout the time-course. Importantly, the marker genes for salicylic acid, jasmonic acid and ET responses, PR1 and PDF1.2 were found to be constitutively upregulated in the 35S::G28 transgenic plants, suggesting that these genes could be the downstream targets for the activity of G28 (a similar constitutive expression pattern of PR1 and PDF1.2 was observed following microarray analysis of the 35S::G28 transgenics). In fact, PDF1.2 has a GCC-box element in its promoter and is therefore potentially a direct target of G28.

Although G28 transcription was activated in response to ethylene, overexpression of G28 had no effect on the well-studied ethylene response pathway that is involved in a variety of developmental responses, including the so-called triple response of seedlings. That is, transgenic plants over-expressing G28 exhibited a normal triple response. The latter observation supports the conclusion that G28 functions specifically in a defense-response pathway.

Transgenic plants that over-expressed G28 and had enhanced resistance to Erysiphe orontii, Sclerotinia sclerotiorum, and Botrytis cinerea are shown. Three independent CAMV 35S promoter::G28 transgenic lines, -15, -10 and -11, were found to be more tolerant to infection with a moderate dose of the fungal pathogen Erysiphe orontii. Erysiphe spores were obtained from 10 to 14 day old Erysiphe cultures, and inoculations were performed by tapping conidia from 1 to 2 heavily infected leaves onto the mesh cover of a settling tower, brushing the mesh with a camel's hair paint brush to break up the conidial chains, and letting the conidia settle for 10 minutes. Plants were 4 to 4.5 weeks old at the time of inoculation. The mesh had a pore size of 95 microns; the settling towers were 28″ high, and were wide enough to fit over a box of plants (6″×6″ or 6″×8″). Symptoms were evaluated 7-21 days post-inoculation.

Enhanced resistance of 35S::G28-15 to the fungal pathogen Sclerotinia sclerotiorum was also observed. Sclerotinia sclerotiorum hyphal cultures were grown in potato dextrose broth. One gram of hyphae is ground, filtered, spun down and resuspended in sterile water. A 1:10 dilution was used to spray four week-old plants grown under a 12 hour light/dark. Two of three independent 35::G28 transgenic lines and infected with Sclerotinia sclerotiorum demonstrated a significant reduction in disease severity as compared to wild-type controls similarly infected.

Enhanced resistance of 35S::G28-15 overexpressing plants to the fungal pathogen Botrytis cinerea was also observed. Botrytis cinerea was grown on potato dextrose agar. A spore culture was made by spreading 10 ml of sterile water on the fungus plate, swirling and transferring spores to 10 ml of sterile water. The spore inoculum (10⁵ spores/ml) was used to spray four week-old plants grown under 12 hour light/dark conditions. Two of three independent 35::G28 transgenic lines infected with Botrytis cinerea showed a significant reduction in disease severity as compared to wild-type controls similarly infected.

G28 overexpression did not seem to have detrimental effects on plant growth or vigor, since plants from most of the lines were morphologically wild-type. In addition, no difference was detected between those lines and the corresponding wild-type controls in all the biochemical assays that were performed.

Table 7 summarizes subsequent experiments and shows the observed trait and response of transgenic 35S::G28 Arabidopsis plants overexpressing G28 when treated with different plant pathogens over particular time periods when inoculated with a plant pathogen (Botrytis, Sclerotinia, or Erysiphe). The first column shows the trait or response category to be analyzed (Response Category); the second column shows the conditions used for the assay (Assay Type and Medium); the third column shows the pathogen species inoculated onto the plant (Description of Pathogen); the fourth column shows the resulting response of the inoculated transgenic plant to the pathogen (Results of Inoculation with Pathogen of Transgenic Arabidopsis Plants). Transgenic Arabidopsis plants overexpressing G28 under the control of the CaMV 35S promoter were found to be more tolerant to pathogens when inoculated with Botrytis, Erysiphe, or Sclerotinia, compared with wild type control plant similarly treated.

TABLE 7
Results of pathogen challenge on Transgenic Arabidopsis plants

| Assay Type | | Results of Inoculation with Pathogen |
|---|---|---|
| and | Description | of Transgenic Arabidopsis |
| Medium | of Pathogen | plants |
| Growth/Plate | Botrytis | 35S::G28: More tolerant |
| Growth/Plate | Sclerotinia | 35S::G28: More tolerant |
| Growth/Plate | Botrytis | 35S::G28: Repeat experiment: Individual |
| | | lines: More tolerant |
| Growth/Plate | Sclerotinia | 35S::G28: Repeat experiment: Individual |
| | | lines: More tolerant |
| Growth/Soil | Erysiphe | 35S::G28: Less fungal growth on 8 out of 9 |
| | | plants. |
| Growth/Soil | Erysiphe | 35S::G28: Repeat experiment: Individual |
| | | lines. Less fungal growth on plants from |
| | | all 3 lines |

Transgenic Arabidopsis plants over-expressing SEQ ID NO:1 (plant G28-11) were more tolerant to pathogens and had less fungal growth when inoculated with Erysiphe orontii compared with wild type control plants (plant Col) similarly treated. Leaves from a transgenic Arabidopsis plant over-expressing SEQ ID NO:1 (leaves G28-11) had less fungal growth when inoculated with Erysiphe orontii compared with wild type control plant (leaves Col) similarly treated.

Transgenic Arabidopsis seedlings over-expressing SEQ ID NO:1 (seedlings G28-15) were more tolerant to pathogen and had more vigorous growth five days following inoculation with Sclerotinia sclerotiorum compared with control seedlings transformed with only the pMEN65 vector (seedlings PMen65) and similarly inoculated with Sclerotinia. Control seedlings were engulfed with fungal hyphae whereas the transgenic seedlings comprising SEQ ID NO: 1 (G28) were tolerant to the presence of hyphae and continued to grow.

Table 8 shows the increased levels of G28 (SEQ ID NO:1), and G1006 (SEQ ID NO: 3), and G1004 (SEQ ID NO: 5) in transgenic 35S::G28 Arabidopsis plants overexpressing G28 when treated with different plant pathogens or methyl jasmonate over particular time periods. The results were determined by microarray analysis using a proprietary Arabidopsis microarray chip. The first column indicates the type of treatment. Columns two through four show the fold increase of the endogenous transcribed polynucleotide levels compared with endogenous levels of an untreated control plant sample, untreated control sample fold levels normalized to 1.00; the second column shows the fold increase of SEQ ID NO: 1 (G28); the third column shows the fold increase of SEQ ID NO: 3 (G1006); the fourth column shows the fold increase of SEQ ID NO: 5 (G1004).

TABLE 8
Increase of endogenous transcript in 35S::G28 Arabidopsis plants
overexpressing G28

| | X-fold increase of | | |
|---|---|---|---|
| | endogenous transcript* | | |
| | G28 | G1006 | G1004 |
| | SEQ ID | SEQ ID | SEQ ID |
| Treatment | NO: 1 | NO: 3 | NO: 5 |
| Botrytis 12 hours | 2.61 | 2.57 | 3.34 |
| Fusarium 24 hours | 3.08 | 3.45 | 1.83 |
| Fusarium 48 hours | 2.33 | 1.95 | 1.54 |
| Erysiphe 7 days | 2.15 | 2.78 | 1.19 |
| Methyl jasmonate 24 hours | 2.26 | 1.71 | 1.03 |
| 35S::G28 & Botrytis 2 hours | 1.43 | 1.37 | 2.17 |
| 35S::G28 & Botrytis 12 hours | 9.99 | 5.55 | 1.62 |
| 35S::G28 & Botrytis 48 hours | 1.37 | 1.5 | 2.44 |

*(control X = 1.00)

Novel Utilities Based on Functional Observations. G28 (AtERF1; SEQ ID NO: 2) was shown to be a key regulator of the plant defense response by overexpressing AtERF1 in transgenic Arabidopsis plants. In these experiments, this gene was shown to provide enhanced resistance to different economically important fungal pathogens, including Erysiphe orontii, Botrytis cinerea, Fusarium oxysporum and Sclerotinia sclerotiorum. Erysiphe species or so-called powdery mildews are obligate biotrophs and will only grow on healthy leaves. Botrytis and Sclerotinia are necrotrophic pathogens that kill host cells to extract nutrients. Fusarium oxysporum, a necrotrophic fungal pathogen, was chosen because unlike the aforementioned fungal pathogens that are foliar pathogens, F. oxysporum primarily infects roots. F. oxysporum is a vascular pathogen causing a variety of disease symptoms including chlorosis (yellowing), stunting, wilting, and root rot, head blight of wheat and barley. Fusarium species also synthesize a wide range of phytotoxic compounds, including the sphinganine analogue mycotoxins.

It was surprising that over expression of a single transcription factor led to enhanced resistance against all three of these fungal pathogens.

Therefore, G28 or its equivalogs can be used to manipulate the defense response in order to generate pathogen-resistant plants. Furthermore, a unique motif, Motif Y (SEQ ID NO: 55) was discovered in G28 orthologs in monocots, but not in dicots, upstream of the conserved AP2 domain of G28. This motif is likely conserved because it functions in a disease tolerance-inducing capacity, and thus monocot-derived G28 equivalogs that comprise Motif Y may be used to enhance disease tolerance in monocots.

### Example IX

### Identification of Homologous Sequences by Computer Homology Search

This example describes identification of genes that are orthologous to Arabidopsis thaliana transcription factors from a computer homology search.

Homologous sequences, including those of paralogs and orthologs from Arabidopsis and other plant species, were identified using database sequence search tools, such as the Basic Local Alignment Search Tool (BLAST; Altschul et al. (1990) supra; and Altschul et al. (1997) Nucleic Acid Res. 25: 3389-3402). The tblastx sequence analysis programs were employed using the BLOSUM-62 scoring matrix (Henikoff and Henikoff (1992) Proc. Natl. Acad. Sci. USA 89: 10915-10919). The entire NCBI GenBank database was filtered for sequences from all plants except Arabidopsis thaliana by selecting all entries in the NCBI GenBank database associated with NCBI taxonomic ID 33090 (Viridiplantae; all plants) and excluding entries associated with taxonomic ID 3701 (Arabidopsis thaliana).

These sequences are compared to sequences representing transcription factor genes presented in the Sequence Listing, using the Washington University TBLASTX algorithm (version 2.0a19MP) at the default settings using gapped alignments with the filter “off”. For each transcription factor gene in the Sequence Listing, individual comparisons were ordered by probability score (P-value), where the score reflects the probability that a particular alignment occurred by chance. For example, a score of 3.6e-40 is 3.6×10-40. In addition to P-values, comparisons were also scored by percentage identity. Percentage identity reflects the degree to which two segments of DNA or protein are identical over a particular length. Examples of sequences so identified are presented in, for example, Table 2, 6 or 7. Paralogous or orthologous sequences were readily identified and available in GenBank by GenBank Accession Number or Test Sequence Annotation (e.g., see Table 6;). The percent sequence identity among these sequences can be as low as 47%, or even lower sequence identity.

Candidate paralogous sequences were identified among Arabidopsis transcription factors through alignment, identity, and phylogenic relationships. G1006 (SEQ ID NO: 4), a paralog of G28, may be found in the Sequence Listing.

Candidate orthologous sequences were identified from proprietary unigene sets of plant gene sequences in Zea mays, Glycine max and Oryza sativa based on significant homology to Arabidopsis transcription factors. These candidates were reciprocally compared to the set of Arabidopsis transcription factors. If the candidate showed maximal similarity in the protein domain to the eliciting transcription factor or to a paralog of the eliciting transcription factor, then it was considered to be an ortholog. Identified non-Arabidopsis sequences that were shown in this manner to be orthologous to the Arabidopsis sequences are provided in, for example, Tables 2, 6 and 7.

### Example X

### Identification of Orthologous and Paralogous Sequences by PCR

Orthologs to Arabidopsis genes may identified by several methods, including hybridization, amplification, or bioinformatically. This example describes how one may identify equivalogs to the Arabidopsis AP2 family transcription factor CBF1 (polynucleotide SEQ ID NO: 45, encoded polypeptide SEQ ID NO: 46), which confers tolerance to abiotic stresses (Thomashow et al. (2002) U.S. Pat. No. 6,417,428), and an example to confirm the function of homologous sequences. In this example, orthologs to CBF1 were found in canola (Brassica napus) using polymerase chain reaction (PCR).

Degenerate primers were designed for regions of AP2 binding domain and outside of the AP2 (carboxyl terminal domain):

(SEQ ID NO: 53)

Mol 368 (reverse)
5′- CAY CCN ATH TAY MGN GGN GT -3′

(SEQ ID NO: 54)

Mol 378 (forward)
5′- GGN ARN ARC ATh CCY TCN GCC -3′

(Y: C/T, N: A/C/G/T, H: A/C/T, M: A/C, R: A/G)

Primer Mol 368 is in the AP2 binding domain of CBF1 (amino acid sequence: His-Pro-Ile-Tyr-Arg-Gly-Val) while primer Mol 378 is outside the AP2 domain (carboxyl terminal domain; amino acid sequence: Met-Ala-Glu-Gly-Met-Leu-Leu-Pro).

The genomic DNA isolated from B. napus was PCR-amplified by using these primers following these conditions: an initial denaturation step of 2 minutes at 93° C.; 35 cycles of 93° C. for 1 minute, 55° C. for 1 minute, and 72° C. for 1 minute; and a final incubation of 7 minutes at 72° C. at the end of cycling.

The PCR products were separated by electrophoresis on a 1.2% agarose gel and transferred to nylon membrane and hybridized with the AT CBF1 probe prepared from Arabidopsis genomic DNA by PCR amplification. The hybridized products were visualized by colorimetric detection system (Boehlinger Mannheim) and the corresponding bands from a similar agarose gel were isolated using the Qiagen Extraction Kit (Qiagen, Valencia Calif.). The DNA fragments were ligated into the TA clone vector from TOPO TA Cloning Kit (Invitrogen Corporation, Carlsbad Calif.) and transformed into E. coli strain TOP10 (Invitrogen).

Seven colonies were picked and the inserts were sequenced on an ABI 377 machine from both strands of sense and antisense after plasmid DNA isolation. The DNA sequence was edited by sequencer and aligned with the AtCBF1 by GCG software and NCBI blast searching.

The nucleic acid sequence and amino acid sequence of one canola ortholog found in this manner (bnCBF1; polynucleotide SEQ ID NO: 51 and polypeptide SEQ ID NO: 52) identified by this process is shown in the Sequence Listing.

The aligned amino acid sequences show that the bnCBF1 gene has 88% identity with the Arabidopsis sequence in the AP2 domain region and 85% identity with the Arabidopsis sequence outside the AP2 domain when aligned for two insertion sequences that are outside the AP2 domain.

Similarly, paralogous sequences to Arabidopsis genes, such as CBF1, may also be identified.

Two paralogs of CBF1 from Arabidopsis thaliana: CBF2 and CBF3. CBF2 and CBF3 have been cloned and sequenced as described below. The sequences of the DNA SEQ ID NO: 47 and 49 and encoded proteins SEQ ID NO: 48 and 50 are set forth in the Sequence Listing.

A lambda cDNA library prepared from RNA isolated from Arabidopsis thaliana ecotype Columbia (Lin and Thomashow (1992) Plant Physiol. 99: 519-525) was screened for recombinant clones that carried inserts related to the CBF1 gene (Stockinger et al. (1997) Proc. Natl. Acad. Sci. USA 94:1035-1040). CBF1 was ³²P-radiolabeled by random priming (Sambrook et al. (1989) supra) and used to screen the library by the plaque-lift technique using standard stringent hybridization and wash conditions (Hajela et al. (1990) Plant Physiol. 93:1246-1252; Sambrook et al. (1989) supra) 6×SSPE buffer, 60° C. for hybridization and 0.1×SSPE buffer and 60° C. for washes). Twelve positively hybridizing clones were obtained and the DNA sequences of the cDNA inserts were determined. The results indicated that the clones fell into three classes. One class carried inserts corresponding to CBF1. The two other classes carried sequences corresponding to two different homologs of CBF1, designated CBF2 and CBF3. The nucleic acid sequences and predicted protein coding sequences for Arabidopsis CBF1, CBF2 and CBF3 are listed in the Sequence Listing (SEQ ID NOs: 45, 47, 49 and SEQ ID NOs: 46, 48, 50, respectively). The nucleic acid sequences and predicted protein coding sequence for Brassica napus CBF ortholog is listed in the Sequence Listing (SEQ ID NOs: 51 and 52, respectively).

A comparison of the nucleic acid sequences of Arabidopsis CBF1, CBF2 and CBF3 indicate that they are 83 to 85% identical as shown in Table 9.

TABLE 9
Identity comparison of Arabidopsis CBF1, CBF2 and CBF3

Percent identity^{a}

| | DNA^{b} | Polypeptide |
|---|---|---|
| cbf1/cbf2 | 85 | 86 |
| cbf1/cbf3 | 83 | 84 |
| cbf2/cbf3 | 84 | 85 |

^{a}Percent identity was determined using the Clustal algorithm from the Megalign program (DNASTAR, Inc.).
^{b}Comparisons of the nucleic acid sequences of the open reading frames are shown.

Similarly, the amino acid sequences of the three CBF polypeptides range from 84 to 86% identity. An alignment of the three amino acidic sequences reveals that most of the differences in amino acid sequence occur in the acidic C-terminal half of the polypeptide. This region of CBF1 serves as an activation domain in both yeast and Arabidopsis (not shown).

Residues 47 to 106 of CBF1 correspond to the AP2 domain of the protein, a DNA binding motif that to date, has only been found in plant proteins. A comparison of the AP2 domains of CBF1, CBF2 and CBF3 indicates that there are a few differences in amino acid sequence. These differences in amino acid sequence might have an effect on DNA binding specificity.

### Example XI

### Transformation of Canola with a Plasmid Containing CBF1, CBF2, or CBF3

After identifying homologous genes to CBF1, canola was transformed with a plasmid containing the Arabidopsis CBF1, CBF2, or CBF3 genes cloned into the vector pGA643 (An (1987) Methods Enzymol. 253: 292). In these constructs the CBF genes were expressed constitutively under the CaMV 35S promoter. In addition, the CBF1 gene was cloned under the control of the Arabidopsis COR15 promoter in the same vector pGA643. Each construct was transformed into Agrobacterium strain GV3101. Transformed Agrobacteria were grown for 2 days in minimal AB medium containing appropriate antibiotics.

Spring canola (B. napus cv. Westar) was transformed using the protocol of Moloney et al. (1989) Plant Cell Reports 8: 238, with some modifications as described. Briefly, seeds were sterilized and plated on half strength MS medium, containing 1% sucrose. Plates were incubated at 24° C. under 60-80 μE/m²s light using a 16 hour light/8 hour dark photoperiod. Cotyledons from 4-5 day old seedlings were collected, the petioles cut and dipped into the Agrobacterium solution. The dipped cotyledons were placed on co-cultivation medium at a density of 20 cotyledons/plate and incubated as described above for 3 days. Explants were transferred to the same media, but containing 300 mg/l timentin (SmithKline Beecham, Pa.) and thinned to ten cotyledons/plate. After 7 days explants were transferred to Selection/Regeneration medium. Transfers were continued every 2-3 weeks (2 or 3 times) until shoots had developed. Shoots were transferred to Shoot-Elongation medium every 2-3 weeks. Healthy looking shoots were transferred to rooting medium. Once good roots had developed, the plants were placed into moist potting soil.

The transformed plants were then analyzed for the presence of the NPTII gene/kanamycin resistance by ELISA, using the ELISA NPTII kit from 5Prine-3Prime Inc. (Boulder, Colo.). Approximately 70% of the screened plants were NPTII positive. Only those plants were further analyzed.

From Northern blot analysis of the plants that were transformed with the constitutively expressing constructs, showed expression of the CBF genes and all CBF genes were capable of inducing the Brassica napus cold-regulated gene BN115 (homolog of the Arabidopsis COR15 gene). Most of the transgenic plants appear to exhibit a normal growth phenotype. As expected, the transgenic plants are more freezing tolerant than the wild-type plants. Using the electrolyte leakage of leaves test, the control showed a 50% leakage at −2° to −3° C. Spring canola transformed with either CBF1 or CBF2 showed a 50% leakage at −6° to −7° C. Spring canola transformed with CBF3 shows a 50% leakage at about −10° to −15° C. Winter canola transformed with CBF3 may show a 50% leakage at about −160 to −20° C. Furthermore, if the spring or winter canola are cold acclimated the transformed plants may exhibit a further increase in freezing tolerance of at least −2° C.

To test salinity tolerance of the transformed plants, plants were watered with 150 mM NaCl. Plants overexpressing CBF1, CBF2, or CBF3 grew better compared with plants that had not been transformed with CBF1, CBF2, or CBF3.

These results demonstrate that equivalogs of Arabidopsis transcription factors can be identified and shown to confer similar functions in non-Arabidopsis plant species.

### Example XII

### Screen of Plant cDNA Library for Sequence Encoding a Transcription Factor DNA Binding Domain and Demonstration of Protein Transcription Regulation Activity

The “one-hybrid” strategy (Li and Herskowitz (1993) Science 262: 1870-1874) is used to screen for plant cDNA clones encoding a polypeptide comprising a transcription factor DNA binding domain, a conserved domain. In brief, yeast strains are constructed that contain a lacZ reporter gene with either wild-type or mutant transcription factor binding promoter element sequences in place of the normal UAS (upstream activator sequence) of the GAL1 promoter. Yeast reporter strains are constructed that carry transcription factor binding promoter element sequences as UAS elements are operably linked upstream (5′) of a lacZ reporter gene with a minimal GAL1 promoter. The strains are transformed with a plant expression library that contains random cDNA inserts fused to the GAL4 activation domain (GAL4-ACT) and screened for blue colony formation on X-gal-treated filters (X-gal: 5-bromo-4-chloro-3-indolyl-β-D-galactoside; Invitrogen Corporation, Carlsbad Calif.). Alternatively, the strains are transformed with a cDNA polynucleotide encoding a known transcription factor DNA binding domain polypeptide sequence.

Yeast strains carrying these reporter constructs produce low levels of beta-galactosidase and form white colonies on filters containing X-gal. The reporter strains carrying wild-type transcription factor binding promoter element sequences are transformed with a polynucleotide that encodes a polypeptide comprising a plant transcription factor DNA binding domain operably linked to the acidic activator domain of the yeast GAL4 transcription factor, “GAL4-ACT”. The clones that contain a polynucleotide encoding a transcription factor DNA binding domain operably linked to GAL4-ACT can bind upstream of the lacZ reporter genes carrying the wild-type transcription factor binding promoter element sequence, activate transcription of the lacZ gene and result in yeast forming blue colonies on X-gal-treated filters.

Upon screening about 2×10⁶ yeast transformants, positive cDNA clones are isolated; i.e., clones that cause yeast strains carrying lacZ reporters operably linked to wild-type transcription factor binding promoter elements to form blue colonies on X-gal-treated filters. The cDNA clones do not cause a yeast strain carrying a mutant type transcription factor binding promoter elements fused to LacZ to turn blue. Thus, a polynucleotide encoding transcription factor DNA binding domain, a conserved domain, is shown to activate transcription of a gene.

### Example XIII

### Gel Shift Assays

The presence of a transcription factor comprising a DNA binding domain that binds to a DNA transcription factor binding element is evaluated using the following gel shift assay. The transcription factor is recombinantly expressed and isolated from E. coli or isolated from plant material. Total soluble protein, including transcription factor, (40 ng) is incubated at room temperature in 10 μl of 1× binding buffer (15 mM HEPES (pH 7.9), 1 mM EDTA, 30 mM KCl, 5% glycerol, 5% bovine serum albumin, 1 mM DTT) plus 50 ng poly(dl-dC):poly(dl-dC; Pharmacia, Piscataway N.J.) with or without 100 ng competitor DNA. After 10 minutes incubation, probe DNA comprising a DNA transcription factor binding element (1 ng) that has been ³²P-labeled by end-filling (Sambrook et al. (1989) supra) is added and the mixture incubated for an additional 10 minutes. Samples are loaded onto polyacrylamide gels (4% w/v) and fractionated by electrophoresis at 150V for 2 h (Sambrook et al. (1989) supra). The degree of transcription factor-probe DNA binding is visualized using autoradiography. Probes and competitor DNAs are prepared from oligonucleotide inserts ligated into the BamHI site of pUC118 (Vieira et al. (1987) Methods Enzymol. 153: 3-11). Orientation and concatenation number of the inserts are determined by dideoxy DNA sequence analysis (Sambrook et al. (1989) supra). Inserts are recovered after restriction digestion with EcoRI and HindIII and fractionation on polyacrylamide gels (12% w/v; Sambrook et al. (1989) supra).

### Example XIV

### Cloning of Transcription Factor Promoters

Promoters are isolated from transcription factor genes that have gene expression patterns useful for a range of applications, as determined by methods well known in the art (including transcript profile analysis with cDNA or oligonucleotide microarrays, Northern blot analysis, semi-quantitative or quantitative RT-PCR). Interesting gene expression profiles are revealed by determining transcript abundance for a selected transcription factor gene after exposure of plants to a range of different experimental conditions, and in a range of different tissue or organ types, or developmental stages. Experimental conditions to which plants are exposed for this purpose includes cold, heat, drought, osmotic challenge, varied hormone concentrations (ABA, GA, auxin, cytokinin, salicylic acid, brassinosteroid), pathogen and pest challenge. The tissue types and developmental stages include stem, root, flower, rosette leaves, cauline leaves, siliques, germinating seed, and meristematic tissue. The set of expression levels provides a pattern that is determined by the regulatory elements of the gene promoter.

Transcription factor promoters for the genes disclosed herein are obtained by cloning 1.5 kb to 2.0 kb of genomic sequence immediately upstream of the translation start codon for the coding sequence of the encoded transcription factor protein. This region includes the 5′-UTR of the transcription factor gene, which can comprise regulatory elements. The 1.5 kb to 2.0 kb region is cloned through PCR methods, using primers that include one in the 3′ direction located at the translation start codon (including appropriate adaptor sequence), and one in the 5′ direction located from 1.5 kb to 2.0 kb upstream of the translation start codon (including appropriate adaptor sequence). The desired fragments are PCR-amplified from Arabidopsis Col-0 genomic DNA using high-fidelity Taq DNA polymerase to minimize the incorporation of point mutation(s). The cloning primers incorporate two rare restriction sites, such as Not1 and Sfi1, found at low frequency throughout the Arabidopsis genome. Additional restriction sites are used in the instances where a Not1 or Sfi1 restriction site is present within the promoter.

The 1.5-2.0 kb fragment upstream from the translation start codon, including the 5′-untranslated region of the transcription factor, is cloned in a binary transformation vector immediately upstream of a suitable reporter gene, or a transactivator gene that is capable of programming expression of a reporter gene in a second gene construct. Reporter genes used include green fluorescent protein (and related fluorescent protein color variants), beta-glucuronidase, and luciferase. Suitable transactivator genes include LexA-GAL4, along with a transactivatable reporter in a second binary plasmid (as disclosed in U.S. patent application Ser. No. 09/958,131, incorporated herein by reference). The binary plasmid(s) is transferred into Agrobacterium and the structure of the plasmid confirmed by PCR. These strains are introduced into Arabidopsis plants as described in other examples, and gene expression patterns determined according to standard methods know to one skilled in the art for monitoring GFP fluorescence, beta-glucuronidase activity, or luminescence.

### Example XV

### Transformation of Dicots

Transcription factor sequences listed in the Sequence Listing recombined into pMEN20 or pMEN65 expression vectors are transformed into a plant for the purpose of modifying plant traits. The cloning vector may be introduced into a variety of cereal plants by means well known in the art such as, for example, direct DNA transfer or Agrobacterium tumefaciens-mediated transformation. It is now routine to produce transgenic plants using most dicot plants (see Weissbach and Weissbach, (1989) supra; Gelvin et al. (1990) supra; Herrera-Estrella et al. (1983) supra; Bevan (1984) supra; and Klee (1985) supra). Methods for analysis of traits are routine in the art and examples are disclosed above.

Numerous protocols for the transformation of tomato and soy plants have been previously described, and are well known in the art. Gruber et al. ((1993) in Methods in Plant Molecular Biology and Biotechnology, p. 89-119, Glick and Thompson, eds., CRC Press, Inc., Boca Raton) describe several expression vectors and culture methods that may be used for cell or tissue transformation and subsequent regeneration. For soybean transformation, methods are described by Miki et al. (1993) in Methods in Plant Molecular Biology and Biotechnology, p. 67-88, Glick and Thompson, eds., CRC Press, Inc., Boca Raton; and U.S. Pat. No. 5,563,055, (Townsend and Thomas), issued Oct. 8, 1996.

There are a substantial number of alternatives to Agrobacterium-mediated transformation protocols, other methods for the purpose of transferring exogenous genes into soybeans or tomatoes. One such method is microprojectile-mediated transformation, in which DNA on the surface of microprojectile particles is driven into plant tissues with a biolistic device (see, for example, Sanford et al., (1987) Part. Sci. Technol. 5:27-37; Christou et al. (1992) Plant. J. 2: 275-281; Sanford (1993) Methods Enzymol. 217: 483-509; Klein et al. (1987) Nature 327: 70-73; U.S. Pat. No. 5,015,580 (Christou et al), issued May 14, 1991; and U.S. Pat. No. 5,322,783 (Tomes et al.), issued Jun. 21, 1994.

Alternatively, sonication methods (see, for example, Zhang et al. (1991) Bio/Technology 9: 996-997); direct uptake of DNA into protoplasts using CaCl₂ precipitation, polyvinyl alcohol or poly-L-ornithine (see, for example, Hain et al. (1985) Mol. Gen. Genet. 199: 161-168; Draper et al., Plant Cell Physiol. 23: 451-458 (1982)); liposome or spheroplast fusion (see, for example, Deshayes et al. (1985) EMBO J., 4: 2731-2737; Christou et al. (1987) Proc. Natl. Acad. Sci. U.S.A. 84: 3962-3966); and electroporation of protoplasts and whole cells and tissues (see, for example, Donn et al. (1990) in Abstracts of VIIth International Congress on Plant Cell and Tissue Culture IAPTC, A2-38: 53; DHalluin et al. (1992) Plant Cell 4: 1495-1505; and Spencer et al. (1994) Plant Mol. Biol. 24: 51-61) have been used to introduce foreign DNA and expression vectors into plants.

After a plant or plant cell is transformed (and the latter regenerated into a plant), the transformed plant may be crossed with itself or a plant from the same line, a non-transformed or wild-type plant, or another transformed plant from a different transgenic line of plants. Crossing provides the advantages of producing new and often stable transgenic varieties. Genes and the traits they confer that have been introduced into a tomato or soybean line may be moved into distinct line of plants using traditional backcrossing techniques well known in the art. Transformation of tomato plants may be conducted using the protocols of Koornneef et al (1986) In Tomato Biotechnology: Alan R. Liss, Inc., 169-178, and in U.S. Pat. No. 6,613,962, the latter method described in brief here. Eight day old cotyledon explants are precultured for 24 hours in Petri dishes containing a feeder layer of Petunia hybrida suspension cells plated on MS medium with 2% (w/v) sucrose and 0.8% agar supplemented with 10 μM α-naphthalene acetic acid and 4.4 μM 6-benzylaminopurine. The explants are then infected with a diluted overnight culture of Agrobacterium tumefaciens containing an expression vector comprising a polynucleotide of the invention for 5-10 minutes, blotted dry on sterile filter paper and cocultured for 48 hours on the original feeder layer plates. Culture conditions are as described above. Overnight cultures of Agrobacterium tumefaciens are diluted in liquid MS medium with 2% (w/v/) sucrose, pH 5.7) to an OD₆₀₀ of 0.8.

Following cocultivation, the cotyledon explants are transferred to Petri dishes with selective medium comprising MS medium with 4.56 μM zeatin, 67.3 μM vancomycin, 418.9 μM cefotaxime and 171.6 μM kanamycin sulfate, and cultured under the culture conditions described above. The explants are subcultured every three weeks onto fresh medium. Emerging shoots are dissected from the underlying callus and transferred to glass jars with selective medium without zeatin to form roots. The formation of roots in a kanamycin sulphate-containing medium is a positive indication of a successful transformation.

Transformation of soybean plants may be conducted using the methods found in, for example, U.S. Pat. No. 5,563,055 (Townsend et al., issued Oct. 8, 1996), described in brief here. In this method soybean seed is surface sterilized by exposure to chlorine gas evolved in a glass bell jar. Seeds are germinated by plating on 1/10 strength agar solidified medium without plant growth regulators and culturing at 28° C. with a 16 hour day length. After three or four days, seed may be prepared for cocultivation. The seedcoat is removed and the elongating radicle removed 3-4 mm below the cotyledons.

Overnight cultures of Agrobacterium tumefaciens harboring the expression vector comprising a polynucleotide of the invention are grown to log phase, pooled, and concentrated by centrifugation. Inoculations are conducted in batches such that each plate of seed was treated with a newly resuspended pellet of Agrobacterium. The pellets are resuspended in 20 ml inoculation medium. The inoculum is poured into a Petri dish containing prepared seed and the cotyledonary nodes are macerated with a surgical blade. After 30 minutes the explants are transferred to plates of the same medium that has been solidified. Explants are embedded with the adaxial side up and level with the surface of the medium and cultured at 22° C. for three days under white fluorescent light. These plants may then be regenerated according to methods well established in the art, such as by moving the explants after three days to a liquid counter-selection medium (see U.S. Pat. No. 5,563,055).

The explants may then be picked, embedded and cultured in solidified selection medium. After one month on selective media transformed tissue becomes visible as green sectors of regenerating tissue against a background of bleached, less healthy tissue. Explants with green sectors are transferred to an elongation medium. Culture is continued on this medium with transfers to fresh plates every two weeks. When shoots are 0.5 cm in length they may be excised at the base and placed in a rooting medium.

### Example XVI

### Transformation and Increased Disease Resistance in Monocots

Cereal plants such as, but not limited to, corn, wheat, rice, sorghum, or barley, may also be transformed with the present polynucleotide sequences, including monocot or dicot-derived sequences such as those presented in Table 2, or AP2 transcription factor genes that encode Motif Y (SEQ ID NO: 55) or a subsequence substantially identical to Motif Y, cloned into a vector such as pGA643 and containing a kanamycin-resistance marker, and expressed constitutively under, for example, the CaMV 35S or COR15 promoters. pMEN20 or pMEN65 and other expression vectors may also be used for the purpose of modifying plant traits. For example, pMEN020 may be modified to replace the NptII coding region with the BAR gene of Streptomyces hygroscopicus that confers resistance to phosphinothricin. The KpnI and BglII sites of the Bar gene are removed by site-directed mutagenesis with silent codon changes.

The cloning vector may be introduced into a variety of cereal plants by means well known in the art including direct DNA transfer or Agrobacterium tumefaciens-mediated transformation. The latter approach may be accomplished by a variety of means, including, for example, that of U.S. Pat. No. 5,591,616, in which monocotyledon callus is transformed by contacting dedifferentiating tissue with the Agrobacterium containing the cloning vector.

The sample tissues are immersed in a suspension of 3×10⁻⁹ cells of Agrobacterium containing the cloning vector for 3-10 minutes. The callus material is cultured on solid medium at 25° C. in the dark for several days. The calli grown on this medium are transferred to Regeneration medium. Transfers are continued every 2-3 weeks (2 or 3 times) until shoots develop. Shoots are then transferred to Shoot-Elongation medium every 2-3 weeks. Healthy looking shoots are transferred to rooting medium and after roots have developed, the plants are placed into moist potting soil.

The transformed plants are then analyzed for the presence of the NPTII gene/kanamycin resistance by ELISA, using the ELISA NPTII kit from 5Prime-3Prime Inc. (Boulder, Colo.).

It is also routine to use other methods to produce transgenic plants of most cereal crops (Vasil (1994) Plant Mol. Biol. 25: 925-937) such as corn, wheat, rice, sorghum (Cassas et al. (1993) Proc. Natl. Acad. Sci. USA 90: 11212-11216, and barley (Wan and Lemeaux (1994) Plant Physiol. 104:3748). DNA transfer methods such as the microprojectile method can be used for corn (Fromm et al. (1990) Bio/Technol. 8: 833-839); Gordon-Kamm et al. (1990) Plant Cell 2: 603-618; Ishida (1990) Nature Biotechnol. 14:745-750), wheat (Vasil et al. (1992) Bio/Technol. 10:667-674; Vasil et al. (1993) Bio/Technol. 11:1553-1558; Weeks et al. (1993) Plant Physiol. 102:1077-1084), and rice (Christou (1991) Bio/Technol. 9:957-962; Hiei et al. (1994) Plant J. 6:271-282; Aldemita and Hodges (1996) Planta 199:612-617; and Hiei et al. (1997) Plant Mol. Biol. 35:205-218). For most cereal plants, embryogenic cells derived from immature scutellum tissues are the preferred cellular targets for transformation (Hiei et al. (1997) Plant Mol. Biol. 35:205-218; Vasil (1994) Plant Mol. Biol. 25: 925-937). For transforming corn embryogenic cells derived from immature scutellar tissue using microprojectile bombardment, the A188XB73 genotype is the preferred genotype (Fromm et al. (1990) Bio/Technol. 8: 833-839; Gordon-Kamm et al. (1990) Plant Cell 2: 603-618). After microprojectile bombardment the tissues are selected on phosphinothricin to identify the transgenic embryogenic cells (Gordon-Kamm et al. (1990) Plant Cell 2: 603-618). Transgenic plants are regenerated by standard corn regeneration techniques (Fromm et al. (1990) Bio/Technol. 8: 833-839; Gordon-Kamm et al. (1990) Plant Cell 2: 603-618).

Northern blot analysis, RT-PCR or microarray analysis of the regenerated, transformed plants may be used to show expression of G28-equivalog genes that are capable of inducing disease tolerance. Monocot-derived equivalogs of G28 gene contain Motif Y or a subsequence substantially identical to Motif Y, and are shown to be expressed and thus may confer disease tolerance.

To verify the ability to confer tolerance, mature plants overexpressing a G28 or G3430 equivalog gene, or alternatively, seedling progeny of these plants, may be challenged with any of several disease-causing organisms, including, for example, the fungal pathogens Botrytis, Fusarium, Erysiphe, and Sclerotinia, or bacterial and other pathogens including Pseudomonas syringae, nematodes, mollicutes, parasites, or herbivorous arthropods.

By comparing wild type and transgenic plants similarly treated, the transgenic plants may be shown to have less fungal growth when inoculated with several of the fungal pathogens, or fewer adverse effects from disease caused by Pseudomonas syringae, nematodes, mollicutes, parasites, or herbivorous arthropods.

The transgenic plants may also have greater yield relative to a control plant when both are faced with the same pathogen challenge. Since members of the G28 clade may be tolerant or resistant to multiple pathogens, plants overexpressing a member of the G3430 subclade of the G28 clade of transcription factor polypeptides may present a smaller yield loss than non-transgenic plants when the two types of plants are faced with similar challenges from any of a number of pathogens, including fungal pathogens. The symptoms of yield loss may include defoliation, chlorosis, stunting, lesions, loss of photosynthesis, distortions and necrosis, and thus methods for reducing yield loss may alleviate some or all of these symptoms.

After a monocot plant or plant cell has been transformed (and the latter regenerated into a plant) and shown to have greater tolerance or resistance to pathogens or greater produce yield relative to a control plant, the transformed monocot plant may be crossed with itself or a plant from the same line, a non-transformed or wild-type monocot plant, or another transformed monocot plant from a different transgenic line of plants.

These experiments would demonstrate that members of the G3430 subclade of transcription factor polypeptides can be identified and shown to confer disease tolerance or resistance in monocots, including tolerance or resistance to multiple pathogens.

### Example XVII

### Induction of G28 Orthologs in Various Crop Species, Including Monocots

Real time PCR experiments, performed in the manner of Example VII, have shown that G28 (SEQ ID NO: 2, AtERF1) and its orthologs in Brassica napus (canola; orthologs Bn bh594074, Bn bh454277), Zea mays (corn; ortholog G3661, SEQ ID NO: 12) and Oryza sativa (rice; ortholog G3430, SEQ ID NO: 10) were induced by the disease-related hormone treatments MeJA and SA in the plant species in which they are found, which supports the premise that these sequences have conserved function across monocot and dicot lineages.

These experiments have demonstrated that members of the G28 clade of transcription factor polypeptides and its G3430 subclade have altered expression patterns in response to disease-related treatments, and, similar to G28, can confer disease tolerance or resistance, including in monocots and to multiple pathogens.

All publications and patent applications mentioned in this specification are herein incorporated by reference to the same extent as if each individual publication or patent application was specifically and individually indicated to be incorporated by reference.

The present invention is not limited by the specific embodiments described herein. The invention now being fully described, it will be apparent to one of ordinary skill in the art that many changes and modifications can be made thereto without departing from the spirit or scope of the appended claims. Modifications that become apparent from the foregoing description and accompanying figures fall within the scope of the claims.

What is claimed is:

1. A transgenic monocot plant having greater tolerance than a control plant to at least one pathogen, wherein the transgenic monocot plant comprises a recombinant polynucleotide encoding a polypeptide member of the G3430 subclade of transcription factor polypeptides.

2. The transgenic monocot plant of claim 1, wherein the polypeptide member comprises a Motif Y that is at least 82% identical to SEQ ID NO: 55.

3. The transgenic monocot plant of claim 2, wherein the recombinant polynucleotide encodes a polypeptide comprising SEQ ID NO: 55.

4. The transgenic monocot plant of claim 1, wherein the recombinant polynucleotide hybridizes over its full length to SEQ ID NO: 9 or its complement under stringent conditions; and
wherein the stringent conditions include two wash steps of 6×SSC at 65° C., each step being 10-30 minutes in duration.

5. The transgenic monocot plant of claim 1, wherein the recombinant polynucleotide is operably linked to at least one regulatory element capable of regulating expression of the recombinant polynucleotide when the recombinant polynucleotide is transformed into a plant.

6. The transgenic monocot plant of claim 5, wherein said at least one regulatory element is selected from the group consisting of a promoter, a transcription initiation start site, an RNA processing signal, a transcription termination site, and a polyadenylation signal.

7. The transgenic monocot plant of claim 6, wherein the promoter is constitutive, inducible, or tissue-specific.

8. The transgenic monocot plant of claim 1, wherein the recombinant polynucleotide is incorporated into an expression vector.

9. The transgenic monocot plant of claim 1, wherein the transgenic monocot plant is a plant cell.

10. The transgenic monocot plant of claim 1, wherein the recombinant polynucleotide encodes a polypeptide comprising SEQ ID NO: 10.

11. The transgenic monocot plant of claim 1, wherein the at least one pathogen is at least one fungal pathogen.

12. The transgenic monocot plant of claim 11, wherein the at least one fungal pathogen is selected from the group consisting of Fusarium, Erysiphe, Sclerotinia and Botrytis.

13. The transgenic monocot plant of claim 1, wherein the recombinant polynucleotide comprises a nucleic acid sequence selected from the group consisting of SEQ ID NO: 9, SEQ ID NO: 11, SEQ ID NO: 29, SEQ ID NO: 31, SEQ ID NO: 33, and SEQ ID NO: 35.

14. Seed produced from the transgenic monocot plant according to claim 1.

15. A method for producing a transformed monocot plant having greater tolerance or resistance to at least one pathogen than a control plant, said method comprising:
(a) providing an expression vector comprising:
(i) a polynucleotide sequence encoding a polypeptide comprising a Motif Y that is at least 82% identical to SEQ ID NO: 55; and
(ii) regulatory elements flanking the polynucleotide sequence, said regulatory elements being able to control expression of the polynucleotide sequence in a target monocot plant; and
(b) transforming the target monocot plant with the expression vector to generate a transformed monocot plant that is capable of expressing the polynucleotide sequence;
wherein the expression of the polynucleotide sequence results in the transformed monocot plant with greater tolerance or resistance to the at least one pathogen than the control plant.

16. The method of claim 15, wherein said polynucleotide sequence hybridizes to SEQ ID NO: 9 under the stringent conditions of 6×SSC and 65° C.

17. The method of claim 15, wherein said at least one pathogen is at least one fungal pathogen.

18. The method of claim 17, wherein the at least one fungal pathogen is selected from the group consisting of Botrytis, Fusarium, Erysiphe, and Sclerotinia.

19. The method of claim 15, the method steps further comprising:
(c) selfing or crossing the transformed monocot plant with itself or another monocot plant, respectively, to produce seed; and
(d) growing a progeny monocot plant from the seed;
wherein the progeny monocot plant has greater tolerance or resistance to the at least one pathogen than the control plant.

20. A method for reducing yield loss due to a plant disease in a monocot plant, the method comprising:
(a) providing an expression vector comprising:
(i) a polynucleotide sequence encoding a polypeptide comprising a Motif Y that is at least 82% identical to SEQ ID NO: 55; and
(ii) regulatory elements flanking the polynucleotide sequence, said regulatory elements being able to control expression of the polynucleotide sequence in a target monocot plant; and
(b) transforming the target monocot plant with the expression vector to generate a transformed monocot plant that is capable of expressing the polynucleotide sequence; and
(c) growing the transformed monocot plant;
wherein the expression of the polynucleotide sequence results in the transformed monocot plant having reduced yield loss due to the plant disease when the transformed monocot plant is contacted by at least one pathogen.

21. The method of claim 20, wherein said plant disease is caused by at least one pathogen.

22. The method of claim 21, wherein said at least one pathogen is at least one fungal pathogen.

23. The method of claim 22, wherein the at least one fungal pathogen is selected from the group consisting of Botrytis, Fusarium, Erysiphe, and Sclerotinia.

24. The method of claim 20, wherein the method alleviates one or more disease symptoms selected from the group consisting of defoliation, chlorosis, stunting, lesions, loss of photosynthesis, distortions and necrosis.
